(19) United States
(12) Patent Application Publication
(10) Pub. No.: US 2024/0382521 A1
(43) Pub. Date: Nov. 21, 2024

(54) METHODS OF ENGINEERING IMMUNE CELLS HAVING REDUCED FRATRICIDAL ACTIVITY

(71) Applicant: Baylor College of Medicine, Houston, TX (US)

(72) Inventors: Maksim Mamonkin, Houston, TX (US); Norihiro Watanabe, Houston, TX (US); Feiyan Mo, Houston, TX (US)

(21) Appl. No.: 18/555,333

(22) PCT Filed: Apr. 21, 2022
(86) PCT No.: PCT/US2022/071845

§ 371 Date: Oct. 13, 2023

Related U.S. Application Data
(60) Provisional application No. 63/178,351, filed on Apr. 22, 2021.

(51) Int. Cl. A61K 35/17; A61K 39/00; A61P 35/02; C07K 16/28; C12N 5/0781; C12N 5/0783; C12N 15/86

(52) U.S. Cl. CPC A61K 35/17; A61K 39/4611; A61K 39/4612; A61K 39/4613; A61K 39/4631; A61K 39/464411; A61K 39/464429; A61P 35/02; C07K 16/2806; C07K 16/2896; C12N 5/0635; C12N 5/0636; C12N 5/0646; C12N 15/86; A61K 2239/13; A61K 2239/17; A61K 2239/48

(57) ABSTRACT

Embodiments of the disclosure include methods and compositions related to targeting of antigen-expressing cells with particular engineered antigen receptors expressed by immune cells. In specific embodiments, immune cells specifically engineered to express particular antigen receptor constructs are cultured in the presence of kinase inhibitors and exhibit reduced fratricidal activity compared to immune cells cultured in the absence of kinase inhibitors. In some embodiments, the genetically engineered immune cells having reduced fratricidal activity are used to treat diseases in subjects, and the fratricidal activity of the genetically engineered immune cells is restored in vivo after substantial elimination of the diseased cells, resulting in elimination of the genetically engineered immune cells.

### CROSS-REFERENCE TO RELATED APPLICATIONS

This application claims priority to U.S. Provisional Application Ser. No. 63/178,351, filed Apr. 22, 2021, which is incorporated by reference herein in its entirety.

### BACKGROUND

### I. Field of the Disclosure

Aspects of this disclosure relate, generally, to at least the fields of cell biology, molecular biology, immunology, and medicine, including cancer medicine.

### II. Background

Chimeric antigen receptor (CAR)-modified T-cells are effective in patients with treatment-resistant B-lineage malignancies, in part due to high expression by malignant cells of targetable antigens not expressed by vital tissues. Extending this approach to other antigens is often complicated by “on-target, off-tumor” toxicity, compromising the safety of these treatments. For example, few antigens specific for T-cell leukemia and lymphoma can be targeted without producing substantial damage to normal T-cells, including to CAR T-cells themselves.

Expression of antigen receptors targeting immune cell antigens often lead to fulminant fratricide (self-targeting) of immune cells, which impairs their expansion, promotes rapid cell exhaustion, and results in a cell product with a suboptimal potency. This limitation often requires implementing additional engineering strategies, such as CRISPR/Cas9-mediated gene editing or a sophisticated receptor system to disrupt trafficking or mask the target antigen from external recognition. These manipulations often result in additional toxicities, increase complexity and cost of manufacturing, and sometimes result in a reduced functionality of immune cells lacking expression of a lineage antigen.

The present disclosure, in particular embodiments, concerns methods and compositions concerning functional genetically engineered immune cells for adoptive cell therapy generated without the use of additional immune cell engineering strategies to reduce immune cell activation, differentiation, and/or fratricide of genetically engineered immune cells, impairment of genetically engineered immune cell expansion, and/or rapid genetically engineered immune cell exhaustion.

### SUMMARY

Aspects of the present disclosure are directed to methods and compositions that enhance adoptive cell therapy. In particular embodiments, the methods and compositions enhance adoptive cell therapy by enhancing expansion of the immune cells of the adoptive cell therapy, by protecting the immune cells of the cell therapy, and/or by protecting cells that are not the target of the immune cell therapy.

The disclosure concerns methods and compositions directed to minimizing self-targeting of cultured immune cells expressing fratricidal antigen receptors without the need for additional engineering. Genetically engineered immune cells may be immune cells of any kind with at least one genetically engineered antigen-targeting receptor, for example, one or more chimeric antigen receptors and/or one or more T-cell receptors that comprise at least one activation signaling domain. The method relies on the reversible pharmacological blockade of signaling by genetically engineered receptor(s) using, for example, one or more tyrosine kinase inhibitors. In some embodiments, expanding genetically engineered immune cells in culture in the presence of these compounds minimizes self-directed killing by inhibiting signaling by the genetically engineered receptor. In some embodiments, the cytotoxicity of engineered immune cells is fully regained upon removal of the inhibitors, for example, after administration to a subject in need thereof.

Embodiments of the disclosure include immune cells; T-cells, including alpha-beta T-cells, gamma-delta T-cells, natural killer T-cells, and mucosal associated invariant T cells; natural killer (NK) cells; myeloid cells, including granulocytes and monocytes; B-cells; target antigens; cancer cell antigens; infectious disease antigens; immune disorder antigens; antigen-targeting receptors; chimeric antigen receptors (CARs), including cancer cell antigen-targeting CARs, infectious disease antigen-targeting CARs, and/or immune disorder antigen-targeting CARs; T-cell receptors (TCRs), including cancer cell antigen-targeting TCRs, infectious disease antigen-targeting TCRs, and/or immune disorder antigen-targeting TCRs; genetically engineered immune cells; genetically engineered T-cells; genetically engineered NK cells; genetically engineered myeloid cells; genetically engineered B-cells; immune cell culture; immune cell activation; immune cell expansion; immune cell selection; genetically engineered immune cell culture; genetically engineered immune cell activation; genetically engineered immune cell expansion; genetically engineered immune cell selection; T-cell culture; T-cell activation; T-cell expansion; T-cell selection; genetically engineered T-cell culture; genetically engineered T-cell activation; genetically engineered T-cell expansion; genetically engineered T-cell selection; T-cell culture; NK cell activation; NK cell expansion; NK cell selection; genetically engineered NK cell culture; genetically engineered NK cell activation; genetically engineered NK cell expansion; genetically engineered NK cell selection; myeloid cell activation; myeloid cell expansion; myeloid cell selection; genetically engineered myeloid cell culture; genetically engineered myeloid cell activation; genetically engineered myeloid cell expansion; genetically engineered myeloid cell selection; B-cell activation; B-cell expansion; B-cell selection; genetically engineered B-cell culture; genetically engineered B-cell activation; genetically engineered B-cell expansion; genetically engineered B-cell selection; kinase inhibitors; tyrosine kinase inhibitors; dasatinib; ibrutinib; pp2; pazopanib; gefitinib; polypeptides; nucleic acids; vectors; cells; pharmaceutical compositions; kits; methods for reducing immune cell activation, differentiation, and/or fratricide by genetically engineered immune cells; methods for generating genetically engineered immune cells expressing one or more CARs and/or TCRs, including a population of genetically engineered immune cells expressing one or more CARs and/or TCRs, having reduced immune cell activation, differentiation, and/or fratricidal activity; methods for reducing immune cell activation, differentiation, and/or fratricide by genetically engineered T-cells expressing one or more CARs and/or TCRs; methods for generating genetically engineered T-cells expressing one or more CARs and/or TCRs, including a population of genetically engineered T-cells expressing one or more CARs and/or TCRs, having reduced fratricidal activity; methods for reducing immune cell activation, differentiation, and/or fratricide by genetically engineered NK cells expressing one or more CARs and/or TCRs; methods for generating genetically engineered NK cells expressing one or more CARs and/or TCRs, including a population of genetically engineered NK cells expressing one or more CARs and/or TCRs, having reduced fratricidal activity; methods for reducing immune cell activation, differentiation, and/or fratricide by genetically engineered myeloid cells expressing one or more CARs and/or TCRs; methods for generating genetically engineered myeloid cells expressing one or more CARs and/or TCRs, including a population of genetically engineered myeloid cells expressing one or more CARs and/or TCRs, having reduced fratricidal activity; methods for reducing immune cell activation, differentiation, and/or fratricide by genetically engineered B-cells expressing one or more CARs and/or TCRs; methods for generating genetically engineered B-cells expressing one or more CARs and/or TCRs, including a population of genetically engineered B-cells expressing one or more CARs and/or TCRs, having reduced fratricidal activity; methods for manipulating immune cells to express one or more CARs and/or TCRs; methods for manipulating T-cells to express one or more CARs and/or TCRs; methods for manipulating NK cells to express one or more CARs and/or TCRs; methods for manipulating myeloid cells to express one or more CARs and/or TCRs; methods for manipulating B-cells to express one or more CARs and/or TCRs; methods and compositions for treating, preventing, and/or reducing the severity of cancer; methods and compositions for treating, preventing, and/or reducing the severity of an infectious disease; and methods and compositions for treating, preventing, and/or reducing the severity of an immune disorder.

Methods of the disclosure can include 1, 2, 3, 4, 5, 6, or more of the following steps:
obtaining a sample from a subject; diagnosing a subject as having cancer; diagnosing a subject as having an infectious disease; diagnosing a subject as having an immune disorder; administering immune cells, including a population of immune cells, manipulated to express one or more antigen-targeting receptors, including one or more CARs and/or one or more TCRs, to a subject: T-cells, including a population of T-cells, manipulated to express one or more antigen-targeting receptors, including one or more CARs and/or one or more TCRs, to a subject; NK cells, including a population of NK cells, manipulated to express one or more antigen-targeting receptors, including one or more CARs and/or one or more TCRs, to a subject; myeloid cells, including a population of myeloid cells, manipulated to express one or more antigen-targeting receptors, including one or more CARs and/or one or more TCRs, to a subject: B-cells, including a population of B-cells, manipulated to express one or more antigen-targeting receptors, including one or more CARs and/or one or more TCRs, to a subject; providing an alternative therapy to a subject; and providing two or more types of therapy to a subject:
expanding immune cells, including a population of immune cells, in culture; expanding immune cells, including a population of immune cells, in culture with kinase inhibitors, including one or more TKIs; expanding genetically engineered immune cells, including a population of genetically engineered immune cells, in culture; expanding genetically engineered immune cells, including a population of genetically engineered immune cells, in culture with kinase inhibitors, including one or more TKIs; expanding T-cells, including a population of T-cells, in culture; expanding T-cells, including a population of T-cells, in culture with kinase inhibitors, including one or more TKIs; expanding genetically engineered T-cells, including a population of genetically engineered T-cells, in culture; expanding genetically engineered T-cells, including a population of genetically engineered T-cells, in culture with kinase inhibitors, including one or more TKIs; expanding NK cells, including a population of NK cells, in culture; expanding NK cells, including a population of NK cells, in culture with kinase inhibitors, including one or more TKIs; expanding genetically engineered NK cells, including a population of genetically engineered NK cells, in culture; expanding genetically engineered NK cells, including a population of genetically engineered NK cells, in culture with kinase inhibitors, including one or more TKIs; expanding myeloid cells, including a population of myeloid cells, in culture; expanding myeloid cells, including a population of myeloid cells, in culture with kinase inhibitors, including one or more TKIs; expanding genetically engineered myeloid cells, including a population of genetically engineered myeloid cells, in culture; expanding genetically engineered myeloid cells, including a population of genetically engineered myeloid cells, in culture with kinase inhibitors, including one or more TKIs; expanding B-cells, including a population of B-cells, in culture; expanding B-cells, including a population of B-cells, in culture with kinase inhibitors, including one or more TKIs; expanding genetically engineered B-cells, including a population of genetically engineered B-cells, in culture; expanding genetically engineered B-cells, including a population of genetically engineered B-cells, in culture with kinase inhibitors, including one or more TKIs;
manipulating immune cells, including a population of immune cells, to express one or more antigen-targeting receptors; manipulating immune cells, including a population of immune cells, to express one or more CARs; manipulating immune cells, including a population of immune cells, to express one or more TCRs; activating immune cells, including a population of immune cells, prior to expanding the immune cells or population thereof in culture; activating immune cells, including a population of immune cells, prior to expanding the immune cells or population thereof in culture with kinase inhibitors, including one or more TKIs; activating T-cells, including a population of T-cells, prior to expanding the T-cells or population thereof in culture; activating T-cells, including a population of T-cells, prior to expanding the T-cells or population thereof in culture with kinase inhibitors, including one or more TKIs; activating NK cells, including a population of NK cells, prior to expanding the NK cells or population thereof in culture; activating NK cells, including a population of NK cells, prior to expanding the NK cells or population thereof in culture with kinase inhibitors, including one or more TKIs; activating myeloid cells, including a population of myeloid cells, prior to expanding the myeloid cells or population thereof in culture; activating myeloid cells, including a population of myeloid cells, prior to expanding the myeloid cells or population thereof in culture with kinase inhibitors, including one or more TKIs; activating B-cells, including a population of B-cells, prior to expanding the B-cells or population thereof in culture; activating B-cells, including a population of B-cells, prior to expanding the B-cells or population thereof in culture with kinase inhibitors, including one or more TKIs;
replenishing kinase inhibitors, including one or more TKIs, in a culture of immune cells, including a population of immune cells; replenishing kinase inhibitors, including one or more TKIs, in a culture of genetically engineered immune cells, including a population of genetically engineered immune cells; replenishing kinase inhibitors, including one or more TKIs, in a culture of T-cells, including a population of T-cells; replenishing kinase inhibitors, including one or more TKIs, in a culture of genetically engineered T-cells, including a population of genetically engineered T-cells; replenishing kinase inhibitors, including one or more TKIs, in a culture of NK cells, including a population of NK cells; replenishing kinase inhibitors, including one or more TKIs, in a culture of genetically engineered NK cells, including a population of genetically engineered NK cells; replenishing kinase inhibitors, including one or more TKIs, in a culture of myeloid cells, including a population of myeloid cells; replenishing kinase inhibitors, including one or more TKIs, in a culture of genetically engineered myeloid cells, including a population of genetically engineered myeloid cells; replenishing kinase inhibitors, including one or more TKIs, in a culture of B-cells, including a population of B-cells; replenishing kinase inhibitors, including one or more TKIs, in a culture of genetically engineered B-cells, including a population of genetically engineered B-cells;
depleting kinase inhibitors, including one or more TKIs, in a culture of immune cells, including a population of immune cells; depleting kinase inhibitors, including one or more TKIs, in a culture of genetically engineered immune cells, including a population of genetically engineered immune cells; depleting kinase inhibitors, including one or more TKIs, in a culture of T-cells, including a population of T-cells; depleting kinase inhibitors, including one or more TKIs, in a culture of genetically engineered T-cells, including a population of genetically engineered T-cells; depleting kinase inhibitors, including one or more TKIs, in a culture of NK cells, including a population of NK cells; depleting kinase inhibitors, including one or more TKIs, in a culture of genetically engineered NK cells, including a population of genetically engineered NK cells; depleting kinase inhibitors, including one or more TKIs, in a culture of myeloid cells, including a population of myeloid cells; depleting kinase inhibitors, including one or more TKIs, in a culture of genetically engineered myeloid cells, including a population of genetically engineered myeloid cells; depleting kinase inhibitors, including one or more TKIs, in a culture of B-cells, including a population of B-cells; depleting kinase inhibitors, including one or more TKIs, in a culture of genetically engineered B-cells, including a population of genetically engineered B-cells;
cryopreserving genetically engineered immune cells, including a population of genetically engineered immune cells; cryopreserving genetically engineered immune cells, including a population of genetically engineered immune cells, after depletion of kinase inhibitors, including one or more TKIs, from the culture of genetically engineered immune cells; cryopreserving genetically engineered T-cells, including a population of genetically engineered T-cells; cryopreserving genetically engineered T-cells, including a population of genetically engineered T-cells, after depletion of kinase inhibitors, including one or more TKIs, from the culture of genetically engineered T-cells; cryopreserving genetically engineered NK cells, including a population of genetically engineered NK cells; cryopreserving genetically engineered NK cells, including a population of genetically engineered NK cells, after depletion of kinase inhibitors, including one or more TKIs, from the culture of genetically engineered NK cells; cryopreserving genetically engineered myeloid cells, including a population of genetically engineered myeloid cells; cryopreserving genetically engineered myeloid cells, including a population of genetically engineered myeloid cells, after depletion of kinase inhibitors, including one or more TKIs, from the culture of genetically engineered myeloid cells; cryopreserving genetically engineered B-cells, including a population of genetically engineered B-cells; and cryopreserving genetically engineered B-cells, including a population of genetically engineered B-cells, after depletion of kinase inhibitors, including one or more TKIs, from the culture of genetically engineered B-cells.

Certain embodiments of the disclosure may exclude one or more of the preceding elements and/or steps.

Compositions of the disclosure can include at least 1, 2, 3, 4, 5, or more of the following components: immune cells; T-cells; NK cells; myeloid cells; B-cells antigen-targeting receptors; chimeric antigen receptors (CARs), including cancer cell antigen-targeting CARs, infectious disease antigen-targeting CARs, and/or immune disorder antigen-targeting CARs; T-cell receptors (TCRs), including cancer cell antigen-targeting TCRs, infectious disease antigen-targeting TCRs, and/or immune disorder antigen-targeting TCRs; genetically engineered immune cells; genetically engineered T-cells; genetically engineered NK cells; genetically engineered myeloid cells; genetically engineered B-cells; kinase inhibitors; tyrosine kinase inhibitors; dasatinib; ibrutinib; pp2; pazopanib; gefitinib; cell culture reagents, including but not limited to media and supplements; therapeutic agents; polypeptides; nucleic acids; and vectors.

Disclosed herein, in some aspects, is a composition comprising an effective amount of a population of genetically engineered immune cells comprising one or more chimeric antigen receptors (CARs) and/or T-cell receptors (TCRs), wherein the population of genetically engineered immune cells or a subset thereof express one or more target antigens to which the one or more CARs and/or TCRs specifically bind, wherein signaling by the one or more CARs and/or TCRs upon binding of the one or more target antigens expressed by the population of genetically engineered immune cells or a subset thereof by the one or more CARs and/or TCRs is reduced upon culture of a population of immune cells manipulated to express the one or more CARs and/or TCRs and/or the population of genetically engineered immune cells in the presence of one or more tyrosine kinase inhibitors (TKIs), and wherein a reduction in signaling by the one or more CARs and/or TCRs reduces immune cell activation, differentiation, and/or fratricide by the population of genetically engineered immune cells or a subset thereof compared to genetically engineered immune cells cultured in the absence of the one or more TKIs. In some embodiments of the composition, the composition further comprises a pharmaceutically acceptable carrier.

In some embodiments of the composition, the immune cells comprise T-cells, Natural Killer (NK) cells, myeloid cells, B-cells, or a mixture thereof. In some embodiments of the composition, the immune cells comprise T-cells. In some embodiments of the composition, the immune cells comprise NK cells. In some embodiments of the composition, the immune cells comprise myeloid cells. In some embodiments of the composition, the immune cells comprise B-cells.

In some embodiments of the composition, the one or more target antigens comprise one or more endogenous gene products expressed by the immune cells. In some embodiments of the composition, the one or more target antigens comprise CD2, CD5, CD7, CD4, CD8, CD3, CS1, CD38, CD99, CD30, 4-1BB, OX40, ICOS, CD26, CD6, TIGIT, PD-1, 2B4, LAG-3, MHC-I, MHC-II, peptide-MHC I, peptide-MHC II, Tim3, CTLA-4, CD112R, CD226, CD96, CD80, CD86, CD112, CD155, KIR2, KIR3, LILRB, CD28, CD40L, CD40, BTLA, GITR, VISTA, NKG2D ligands, or CD70.

In some embodiments of the composition, the one or more target antigens comprise one or more antigens acquired via trogocytosis and expressed by the immune cells.

In some embodiments of the composition, the one or more CARs and/or TCRs comprise one or more antibodies or fragments thereof with specificity against the one or more target antigens. In some embodiments of the composition, the antibodies or fragments thereof are scFv monoclonal antibodies, nanobodies/VHH-only sequences, fibronectin-derived binding domains, DARPINs, or natural ligands. In some embodiments of the composition, the one or more CARs comprise a hinge or spacer comprising a sequence derived from IgG, CD3, CD4, CD5, CD8, CD9, CD16, CD22, CD28, CD33, CD37, CD45, CD64, CD80, CD86, CD134, CD137, CD154, 4-1BB, OX40, a T-cell receptor α or β chain, ICOS, or a combination thereof. In some embodiments of the composition, the one or more CARs comprise a hinge or spacer comprising an IgG-derived sequence. In some embodiments of the composition, the one or more CARs comprise a hinge comprising an IgG4-derived sequence. In some embodiments of the composition, the one or more CARs comprise a spacer comprising an IgG1-derived sequence. In some embodiments of the composition, the one or more CARs comprise a C_{H}3 IgG1 spacer. In some embodiments of the composition, the one or more CARs comprise one or more signaling domains from CD2, CD3ξ, CD3δ, CD3ε, CD3γ, Fc receptors, CD79a, CD79b, CLEC-2, CD7, LFA-1 (CD11a/CD18), CD27, CD28, CD30, CD40, 4-1BB (CD137), CD278, 2B4, DNAM-1, OX40, NKG2C, NKG2D, DAP10, DAP12, B7-1/CD80, CD28, 4-1BBL, B7-2/CD86, CTLA-4, B7-H1/PD-L1, ICOS, B7-H2, PD-1, B7-H3, PD-L2, B7-H4, PDCD6, HVEM, LIGHT, ICAM-1, BTLA, GITR, or a combination thereof. In some embodiments of the composition, the one or more CARs comprise one or more signaling domains from CD35, CD28, 4-1BB, or a combination thereof.

In some embodiments of the composition, the one or more CARs and/or TCRs are encoded by one or more isolated nucleic acid sequences. In some embodiments of the composition, the one or more isolated nucleic acid sequences are comprised in one or more expression vectors. In some embodiments of the composition, the one or more expression vectors are a lentiviral vector, a gamma-retroviral vector, adenoviral vector, an adeno-associated viral vector, or a combination thereof.

In some embodiments of the composition, the one or more TKIs comprise one or more Src kinase inhibitors. In some embodiments of the composition, the one or more TKIs comprise dasatinib, ibrutinib, pp2, pazopanib, gefitinib, or a combination thereof. In some embodiments of the composition, at least one of the one or more TKIs comprises dasatinib. In some embodiments of the composition, at least one of the one or more TKIs comprises ibrutinib. In some embodiments of the composition, the one or more TKIs comprise dasatinib and ibrutinib.

In some embodiments of the composition, one or more endogenous genes in the population of genetically engineered immune cells or a subset thereof are not inhibited.

Disclosed herein, in some aspects, is a method of generating a population of genetically engineered immune cells, the method comprising manipulating a population of immune cells in culture with one or more TKIs to express one or more CARs and/or TCRs to produce the genetically engineered immune cells, wherein the produced population of genetically engineered immune cells or a subset thereof have reduced fratricidal activity in culture compared to genetically engineered immune cells cultured in the absence of the one or more TKIs.

In some embodiments of the method, the immune cells comprise T-cells, Natural Killer (NK) cells, myeloid cells, B-cells, or a mixture thereof. In some embodiments of the method, the immune cells comprise T-cells. In some embodiments of the method, the immune cells comprise NK cells. In some embodiments of the composition, the immune cells comprise myeloid cells. In some embodiments of the composition, the immune cells comprise B-cells.

In some embodiments of the method, the population of genetically engineered immune cells or a subset thereof express one or more target antigens to which the one or more CARs and/or TCRs specifically bind. In some embodiments of the method, signaling by the one or more CARs and/or TCRs upon binding of the one or more target antigens expressed by the population of genetically engineered immune cells or a subset thereof by the one or more CARs and/or TCRs is reduced upon culture of the immune cells and the population of genetically engineered immune cells in the presence of the one or more TKIs. In some embodiments of the method, a reduction in signaling by the one or more CARs and/or TCRs reduces immune cell activation, differentiation, and/or fratricide by the population of genetically engineered immune cells or a subset thereof during expansion of the genetically engineered immune cells in culture compared to genetically engineered immune cells cultured in the absence of the one or more TKIs.

In some embodiments of the method, the one or more target antigens comprise one or more endogenous gene products expressed by the immune cells. In some embodiments of the method, the one or more target antigens comprise CD2, CD5, CD7, CD4, CD8, CD3, CS1, CD38, CD99, CD30, 4-1BB, OX40, ICOS, CD26, CD6, TIGIT, PD-1, 2B4, LAG-3, MHC-I, MHC-II, peptide-MHC I, peptide-MHC II, Tim3, CTLA-4, CD112R, CD226, CD96, CD80, CD86, CD112, CD155, KIR2, KIR3, LILRB, CD28, CD40L, CD40, BTLA, GITR, VISTA, NKG2D ligands, or CD70.

In some embodiments of the method, the one or more target antigens comprise one or more antigens acquired via trogocytosis and expressed by the immune cells.

In some embodiments of the method, the one or more CARs and/or TCRs comprise one or more antibodies or fragments thereof with specificity against the one or more target antigens. In some embodiments of the method, the antibodies or fragments thereof are scFv monoclonal antibodies, nanobodies/VHH-only sequences, fibronectin-derived binding domains, DARPINs, or natural ligands. In some embodiments of the method, the one or more CARs comprise a hinge or spacer comprising a sequence derived from IgG, CD3, CD4, CD5, CD8, CD9, CD16, CD22, CD28, CD33, CD37, CD45, CD64, CD80, CD86, CD134, CD137, CD154, 4-1BB, OX40, a T-cell receptor α or β chain, a CD3ξ chain, ICOS, or a combination thereof. In some embodiments of the method, the one or more CARs comprise a hinge comprising an IgG4-derived sequence. In some embodiments of the method, the one or more CARs comprise a spacer comprising an IgG-derived sequence. In some embodiments of the method, the one or more CARs comprise a spacer comprising an IgG1-derived sequence. In some embodiments of the method, the one or more CARs comprise a C_{H}3 IgG1 spacer. In some embodiments of the method, the one or more CARs comprise one or more signaling domains from CD2, CD3ξ, CD3δ, CD3ε, CD3γ, Fc receptors, CD79a, CD79b, CLEC-2, CD7, LFA-1 (CD11a/CD18), CD27, CD28, CD30, CD40, 4-1BB (CD137), CD278, 2B4, DNAM-1, OX40, NKG2C, NKG2D, DAP10, DAP12, B7-1/CD80, CD28, 4-1BBL, B7-2/CD86, CTLA-4, B7-H1/PD-L1, ICOS, B7-H2, PD-1, B7-H3, PD-L2, B7-H4, PDCD6, HVEM, LIGHT, ICAM-1, BTLA, GITR, or a combination thereof. In some embodiments of the method, the one or more CARs comprise one or more signaling domains from CD3ξ, CD28, 4-1BB, or a combination thereof.

In some embodiments of the method, the concentration of each of the one or more TKIs in culture is between 0.01 μM to 10 μM. In some embodiments of the method, the concentration of each of the one or more TKIs in culture is between 0.1 μM to 1 μM. In some embodiments of the method, the one or more TKIs comprise one or more Src kinase inhibitors. In some embodiments of the method, the one or more TKIs comprise dasatinib, ibrutinib, pp2, pazopanib, gefitinib, or a combination thereof. In some embodiments of the method, at least one of the one or more TKIs comprises dasatinib. In some embodiments of the method, at least one of the one or more TKIs comprises ibrutinib. In some embodiments of the method, the one or more TKIs comprise dasatinib and ibrutinib. In some embodiments of the method, the concentration of dasatinib in culture is 0.5 μM. In some embodiments of the method, the concentration of ibrutinib in culture is 0.2 μM.

In some embodiments of the method, the one or more TKIs are added to the culture between 0 to 7 days before manipulation of the immune cell population to express the one or more CARs and/or TCRs. In some embodiments of the method, the one or more TKIs are added to the culture between 0 to 5 days before manipulation of the immune cell population to express the one or more CARs and/or TCRs. In some embodiments of the method, the one or more TKIs are added to the culture between 0 to 3 days before manipulation of the immune cell population to express the one or more CARs and/or TCRs.

In some embodiments of the method, the immune cell population is manipulated to express the one or more CARs and/or TCRs with one or more expression vectors comprising one or more isolated nucleic acid sequences encoding the one or more CARs and/or TCRs. In some embodiments of the method, the one or more expression vectors are a lentiviral vector, a gamma-retroviral vector, adenoviral vector, adeno-associated viral vector, or a combination thereof.

In some embodiments of the method, the method further comprises expanding the population of immune cells in culture with the one or more TKIs prior to manipulating the population of immune cells to express one or more CARs and/or TCRs to produce the population of genetically engineered immune cells. In some embodiments of the method, the method further comprises expanding the population of genetically engineered immune cells in culture with the one or more TKIs after manipulation of the population of immune cells to express one or more CARs and/or TCRs. In some embodiments of the method, the method further comprises activating the population of immune cells prior to manipulating the population of immune cells to express one or more CARs and/or TCRs to produce the population of genetically engineered immune cells.

In some embodiments of the method, the method further comprises replenishing the one or more TKIs in the culture every 1, 2, 3, 4, or 5 days during culture. In some embodiments of the method, the one or more TKIs are replenished in the culture every day during culture. In some embodiments of the method, the one or more TKIs are replenished in the culture every 2 days during culture. In some embodiments of the method, the one or more TKIs are replenished in the culture every 3 days during culture. In some embodiments of the method, the one or more TKIs are replenished in the culture every 4 days during culture. In some embodiments of the method, the one or more TKIs are replenished in the culture every 5 days during culture.

In some embodiments of the method, the method further comprises depleting the population of genetically engineered immune cells of the one or more TKIs between 1 to 21 days after manipulation of the population of immune cells to express one or more CARs and/or TCRs to produce the genetically engineered immune cells. In some embodiments of the method, the population of genetically engineered immune cells is depleted of the one or more between 1 to 14 days after manipulation of the population of immune cells to express one or more CARs and/or TCRs to produce the population of genetically engineered immune cells. In some embodiments of the method, the population of genetically engineered immune cells is depleted of the one or more TKIs between 1 to 7 days after manipulation of the population of immune cells to express one or more CARs and/or TCRs to produce the population of genetically engineered immune cells.

In some embodiments of the method, the population of genetically engineered immune cells is depleted of the one or more TKIs by sequential media washes of the population of genetically engineered immune cells. In some embodiments of the method, 2, 3, 4, 5, or 6 sequential washes of the population of genetically engineered immune cells are performed. In some embodiments of the method, 4 sequential washes of the population of genetically engineered cells are performed.

In some embodiments of the method, the method further comprises cryopreserving the population of genetically engineered cells. In some embodiments of the method, the population of genetically engineered cells are cryopreserved after depleting the population of genetically engineered cells of the one or more TKIs.

In some embodiments of the method, one or more endogenous genes in the immune cells and/or the population of genetically engineered cells or a subset thereof are not inhibited.

Disclosed herein, in some aspects, is a population of genetically engineered immune cells produced by the methods disclosed herein.

Disclosed herein, in some aspects, is a method of killing a diseased cell, the method comprising contacting the diseased cell with a composition disclosed herein or a population of genetically engineered immune cells disclosed herein. In some embodiments, the diseased cell is a cancer cell. In some embodiments, the cancer comprises T-ALL, T-cell lymphoma, leukemia, lymphoma, multiple myeloma, or a solid tumor. In some embodiments, the diseased cell is a cell infected by an infectious disease microorganism. In some embodiments, the diseased cell is a cell affected by an immune disorder.

Disclosed herein, in some aspects, is a method of treating a cancer in a subject, the method comprising administering to a subject in need thereof a therapeutically effective amount of a composition disclosed herein or a population of genetically engineered immune cells disclosed herein, wherein the one or more target antigens to which the one or more CARs and/or TCRs specifically bind are expressed by cancer cells in vivo, wherein the one or more CARs and/or TCRs specifically bind the one or more target antigens expressed by the cancer cells in vivo, and wherein binding of the one or more CARs and/or TCRs to the one or more target antigens expressed by the cancer cells in vivo results in elimination of the cancer cells.

In some embodiments, the amount of genetically engineered immune cells administered to the subject ranges from about 10⁴ up to about 10⁸ cells per kg body weight of the subject. In some embodiments, the composition or the population of genetically engineered immune cells is administered to the subject by infusion, intravenously, intraperitoneally, intratracheally, intramuscularly, endoscopically, percutaneously, subcutaneously, regionally, intracranially, by direct injection, or by perfusion.

In some embodiments, the fratricidal activity of the population of genetically engineered immune cells is restored in vivo after substantial elimination of the cancer cells. In some embodiments, restoration of the fratricidal activity of the population of genetically engineered immune cells results in elimination of the genetically engineered immune cells.

In some embodiments, the cancer is a myeloid malignancy, a lymphoid malignancy, and/or a solid tumor. In some embodiments, the cancer is T-cell acute lymphoblastic leukemia (T-ALL) or T-cell lymphoma.

Disclosed herein, in some aspects, is a method of treating an immune disorder in a subject, the method comprising administering to a subject in need thereof a therapeutically effective amount of a composition disclosed herein or a population of genetically engineered immune cells disclosed herein, wherein the one or more target antigens to which the one or more CARs and/or TCRs specifically bind are expressed by immune cells in vivo, wherein the one or more CARs and/or TCRs specifically bind the one or more target antigens expressed by the immune cells in vivo, and wherein binding of the one or more CARs and/or TCRs to the one or more target antigens expressed by the immune cells in vivo results in elimination of the immune cells.

In some embodiments, the amount of genetically engineered immune cells administered to the subject ranges from about 10⁴ up to about 10⁸ cells per kg body weight of the subject. In some embodiments, the composition or the population of genetically engineered immune cells is administered to the subject by infusion, intravenously, intraperitoneally, intratracheally, intramuscularly, endoscopically, percutaneously, subcutaneously, regionally, intracranially, by direct injection, or by perfusion.

In some embodiments, the fratricidal activity of the population of genetically engineered immune cells is restored in vivo after substantial elimination of the immune cells. In some embodiments, restoration of the fratricidal activity of the population of genetically engineered immune cells results in elimination of the genetically engineered immune cells.

In some embodiments, the immune disorder is an auto- or allo-immune disorder. In some embodiments, the auto- or allo-immune disorder is graft versus host disease, type 1 diabetes, multiple sclerosis, rheumatoid arthritis, psoriatic arthritis, systemic lupus erythematosus, inflammatory bowel disease, Guillain-Barre syndrome, chronic inflammatory demyelinating polyneuropathy, psoriasis, Graves' disease, Hashimoto's thyroiditis, myasthenia gravis, and/or vasculitis.

Disclosed herein, in some aspects, is a composition comprising an effective amount of a population of genetically engineered immune cells comprising one or more chimeric antigen receptors (CARs) and/or T-cell receptors (TCRs), said composition produced by manipulating a population of immune cells in culture with one or more TKIs to express one or more CARs and/or TCRs to produce the population of genetically engineered immune cells, wherein the population of genetically engineered immune cells or a subset thereof express one or more target antigens to which the one or more CARs and/or TCRs specifically bind, wherein signaling by the one or more CARs and/or TCRs upon binding of the one or more target antigens expressed by the population of genetically engineered immune cells or a subset thereof by the one or more CARs and/or TCRs is reduced upon culture of the immune cells manipulated to express the one or more CARs and/or TCRs and/or the population of genetically engineered immune cells in the presence of one or more TKIs, and wherein a reduction in signaling by the one or more CARs and/or TCRs reduces immune cell activation, differentiation, and/or fratricide by the population of genetically engineered immune cells or a subset thereof compared to genetically engineered immune cells cultured in the absence of the one or more TKIs. In some embodiments, the composition comprising an effective amount of a population of genetically engineered immune cells produced by manipulating a population of immune cells in culture with one or more TKIs to express one or more CARs and/or TCRs further comprises a pharmaceutically acceptable carrier

In some embodiments of the composition comprising an effective amount of a population of genetically engineered immune cells produced by manipulating a population of immune cells in culture with one or more TKIs to express one or more CARs and/or TCRs, the immune cells comprise T-cells, Natural Killer (NK) cells, myeloid cells, B-cells, or a mixture thereof. In some embodiments, the immune cells comprise T-cells. In some embodiments, the immune cells comprise NK cells. In some embodiments of the composition, the immune cells comprise myeloid cells. In some embodiments of the composition, the immune cells comprise B-cells.

In some embodiments of the composition comprising an effective amount of a population of genetically engineered immune cells produced by manipulating a population of immune cells in culture with one or more TKIs to express one or more CARs and/or TCRs, the one or more target antigens comprise one or more endogenous gene products expressed by the immune cells. In some embodiments, the one or more target antigens comprise CD2, CD5, CD7, CD4, CD8, CD3, CS1, CD38, CD99, CD30, 4-1BB, OX40, ICOS, CD26, CD6, TIGIT, PD-1, 2B4, LAG-3, MHC-I, MHC-II, peptide-MHC I, peptide-MHC II, Tim3, CTLA-4, CD112R, CD226, CD96, CD80, CD86, CD112, CD155, KIR2, KIR3, LILRB, CD28, CD40L, CD40, BTLA, GITR, VISTA, NKG2D ligands, or CD70.

In some embodiments of the composition comprising an effective amount of a population of genetically engineered immune cells produced by manipulating a population of immune cells in culture with one or more TKIs to express one or more CARs and/or TCRs, the one or more target antigens comprise one or more antigens acquired via trogocytosis and expressed by the immune cells.

In some embodiments of the composition comprising an effective amount of a population of genetically engineered immune cells produced by manipulating a population of immune cells in culture with one or more TKIs to express one or more CARs and/or TCRs, the one or more CARs and/or TCRs comprise one or more antibodies or fragments thereof with specificity against the one or more target antigens. In some embodiments, the antibodies or fragments thereof are scFv monoclonal antibodies, nanobodies/VHH-only sequences, fibronectin-derived binding domains, DARPINs, or natural ligands. In some embodiments, the one or more CARs comprise a hinge or spacer comprising a sequence derived from IgG, CD3, CD4, CD5, CD8, CD9, CD16, CD22, CD28, CD33, CD37, CD45, CD64, CD80, CD86, CD134, CD137, CD154, 4-1BB, OX40, a T-cell receptor α or β chain, a CD3ξ chain, ICOS, or a combination thereof. In some embodiments, the one or more CARs comprise a hinge comprising an IgG4-derived sequence. In some embodiments, the one or more CARs comprise a spacer comprising an IgG-derived sequence. In some embodiments, the one or more CARs comprise a spacer comprising an IgG1-derived sequence. In some embodiments, the one or more CARs comprise a C_{H}3 IgG1 spacer. In some embodiments, the one or more CARs comprise one or more signaling domains from CD2, CD3ξ, CD3δ, CD3ε, CD3γ, Fc receptors, CD79a, CD79b, CLEC-2, CD7, LFA-1 (CD11a/CD18), CD27, CD28, CD30, CD40, 4-1BB (CD137), CD278, 2B4, DNAM-1, OX40, NKG2C, NKG2D, DAP10, DAP12, B7-1/CD80, CD28, 4-1BBL, B7-2/CD86, CTLA-4, B7-H1/PD-L1, ICOS, B7-H2, PD-1, B7-H3, PD-L2, B7-H4, PDCD6, HVEM, LIGHT, ICAM-1, BTLA, GITR, or a combination thereof. In some embodiments, the one or more CARs comprise one or more signaling domains from CD35, CD28, 4-1BB, or a combination thereof.

In some embodiments of the composition comprising an effective amount of a population of genetically engineered immune cells produced by manipulating a population of immune cells in culture with one or more TKIs to express one or more CARs and/or TCRs, the one or more CARs and/or TCRs are encoded by one or more isolated nucleic acid sequences. In some embodiments, the one or more isolated nucleic acid sequences are comprised in one or more expression vectors. In some embodiments, the one or more expression vectors are a lentiviral vector, a gamma-retroviral vector, adenoviral vector, an adeno-associated viral vector, or a combination thereof.

In some embodiments of the composition comprising an effective amount of a population of genetically engineered immune cells produced by manipulating a population of immune cells in culture with one or more TKIs to express one or more CARs and/or TCRs, the concentration of each of the one or more TKIs in culture is between 0.01 μM to 10 μM. In some embodiments, the concentration of each of the one or more TKIs in culture is between 0.1 μM to 1 μM. In some embodiments, the one or more TKIs comprise dasatinib, ibrutinib, pp2, pazopanib, gefitinib, or a combination thereof. In some embodiments, at least one of the one or more TKIs comprises dasatinib. In some embodiments, at least one of the one or more TKIs comprises ibrutinib. In some embodiments, the one or more TKIs comprise dasatinib and ibrutinib. In some embodiments, the concentration of dasatinib in culture is 0.5 μM. In some embodiments, the concentration of ibrutinib in culture is 0.2 μM.

In some embodiments of the composition comprising an effective amount of a population of genetically engineered immune cells produced by manipulating a population of immune cells in culture with one or more TKIs to express one or more CARs and/or TCRs, the one or more TKIs are added to the culture between 0 to 7 days before manipulation of the immune cell population to express the one or more CARs and/or TCRs. In some embodiments, the one or more TKIs are added to the culture between 0 to 5 days before manipulation of the immune cell population to express the one or more CARs and/or TCRs. In some embodiments, the one or more TKIs are added to the culture between 0 to 3 days before manipulation of the immune cell population to express the one or more CARs and/or TCRs.

In some embodiments of the composition comprising an effective amount of a population of genetically engineered immune cells produced by manipulating a population of immune cells in culture with one or more TKIs to express one or more CARs and/or TCRs, the immune cell population is manipulated to express the one or more CARs and/or TCRs with one or more expression vectors comprising one or more isolated nucleic acid sequences encoding the one or more CARs and/or TCRs. In some embodiments, the one or more expression vectors are a lentiviral vector, a gamma-retroviral vector, adenoviral vector, adeno-associated viral vector, or a combination thereof.

In some embodiments of the composition comprising an effective amount of a population of genetically engineered immune cells produced by manipulating a population of immune cells in culture with one or more TKIs to express one or more CARs and/or TCRs, the population of immune cells is expanded in culture with the one or more TKIs prior to manipulating the population of immune cells to express one or more CARs and/or TCRs to produce the population of genetically engineered immune cells. In some embodiments of the composition comprising an effective amount of a population of genetically engineered immune cells produced by manipulating a population of immune cells in culture with one or more TKIs to express one or more CARs and/or TCRs, the population of genetically engineered immune cells is expanded in culture with the one or more TKIs after manipulation of the population of immune cells to express one or more CARs and/or TCRs. In some embodiments of the composition comprising an effective amount of a population of genetically engineered immune cells produced by manipulating a population of immune cells in culture with one or more TKIs to express one or more CARs and/or TCRs, the population of immune cells is activated prior to manipulating the population of immune cells to express one or more CARs and/or TCRs to produce the population of genetically engineered immune cells.

In some embodiments of the composition comprising an effective amount of a population of genetically engineered immune cells produced by manipulating a population of immune cells in culture with one or more TKIs to express one or more CARs and/or TCRs, the one or more TKIs are replenished in the culture every 1, 2, 3, 4, or 5 days during culture. In some embodiments, the one or more TKIs are replenished in the culture every day during culture. In some embodiments, the one or more TKIs are replenished in the culture every 2 days during culture. In some embodiments, the one or more TKIs are replenished in the culture every 3 days during culture. In some embodiments, the one or more TKIs are replenished in the culture every 4 days during culture. In some embodiments, the one or more TKIs are replenished in the culture every 5 days during culture.

In some embodiments of the composition comprising an effective amount of a population of genetically engineered immune cells produced by manipulating a population of immune cells in culture with one or more TKIs to express one or more CARs and/or TCRs, the population of genetically engineered immune cells is depleted of the one or more TKIs between 1 to 21 days after manipulation of the population of immune cells to express one or more CARs and/or TCRs to produce the population of genetically engineered immune cells. In some embodiments, the population of genetically engineered immune cells is depleted of the one or more TKIs between 1 to 14 days after manipulation of the population of immune cells to express one or more CARs and/or TCRs to produce population of the genetically engineered immune cells. In some embodiments, the population of genetically engineered immune cells is depleted of the one or more TKIs between 1 to 7 days after manipulation of the population of immune cells to express one or more CARs and/or TCRs to produce the population of genetically engineered immune cells. In some embodiments, the population of genetically engineered immune cells is depleted of the one or more kinase inhibitors by sequential media washes of the population of genetically engineered immune cells. In some embodiments, 2, 3, 4, 5, or 6 sequential washes of the population of genetically engineered immune cells are performed. In some embodiments, 4 sequential washes of the population of genetically engineered immune cells are performed.

In some embodiments of the composition comprising an effective amount of a population of genetically engineered immune cells produced by manipulating a population of immune cells in culture with one or more TKIs to express one or more CARs and/or TCRs, the population of genetically engineered immune cells is cryopreserved. In some embodiments, the population of genetically engineered immune cells is cryopreserved after depleting the population of genetically engineered immune cells of the one or more TKIs.

In some embodiments of the composition comprising an effective amount of a population of genetically engineered immune cells produced by manipulating a population of immune cells in culture with one or more TKIs to express one or more CARs and/or TCRs, one or more endogenous genes in the immune cells and/or the genetically engineered immune cells are not inhibited.

Any method in the context of a therapeutic, diagnostic, or physiologic purpose or effect may also be described in “use” claim language such as “use of” any compound, composition, or agent discussed herein for achieving or implementing a described therapeutic, diagnostic, or physiologic purpose or effect.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

It is specifically contemplated that any limitation discussed with respect to one embodiment of the disclosure may apply to any other embodiment of the disclosure. Furthermore, any composition of the disclosure may be used in any method of the disclosure, and any method of the disclosure may be used to produce or to utilize any composition of the disclosure. Aspects of an embodiment set forth in the Examples are also embodiments that may be implemented in the context of embodiments discussed elsewhere in a different Example or elsewhere in the application, such as in the Summary, Detailed Description, Claims, and Description of the Drawings.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1I. Chemical inhibition of CAR signaling reduces fratricide and terminal differentiation and improves viability and anti-tumor function of CD5 CAR T-cells. Subset composition (FIG. 1A) and overall expansion (FIG. 1B) of control non-transduced (NT) and CD5 CAR T-cells expanded in the presence of chemical inhibitors dasatinib (Das), pp2, pazopanib (Paz), gefitinib (Gef), and ibrutinib (Ibr). Viability (FIG. 1C) and expansion (FIG. 1D) of CD5 CAR T-cells in the presence of dasatinib (Das) and ibrutinib (Ibr) alone or in combination. FIG. 1E. Overall numbers of minimally differentiated CD5 CAR T-cells. Cytotoxicity of CD5 CAR T-cells after washing inhibitors off against CCRF-CEM (FIG. 1F) and Jurkat (FIG. 1G) leukemic cell lines. FIG. 1H. Anti-tumor activity of CD5 CAR T-cells in mouse xenograft model of aggressive disseminated CD5+ leukemia (CCRF-CEM). FIG. 1I. Overall survival of mice in each experimental group.

FIGS. 2A-2F. Dasatinib and ibrutinib prevents CD7 CAR T-cell fratricide and the inhibitory effect is reversible. FIG. 2A. A schematic diagram showing the effect of dasatinib and ibrutinib on CAR signaling. FIG. 2B. An outline of CAR-T cell generation. FIG. 2C. Representative flow plots (left) and summary of percent of CAR⁺ and CD7⁺ cells (right) on day 4 post transduction for each specified T-cell type (mean±SD, n=4). FIG. 2D. Left: Ex vivo fold expansion of specified T-cell types overtime (mean±SD, n=4). Right: Cell viability on day 4 post transduction determined by flow cytometry (mean±SD, n=4). FIG. 2E. Cytotoxicity of specified effector T-cells against Jurkat (left, mean±SD, n=10) or CCRF-CEM (right, mean±SD, n=6) target cells at 72 hours after coculture setup. FIG. 2F. Expansion of specified effector T-cells when cocultured with Jurkat (left, mean±SD, n=10) or CCRF-CEM (right, mean±SD, n=6) target cells for 72 hours. Statistical differences are calculated by One-way ANOVA with Tukey's multiple comparisons (FIGS. 2C-2F). *p<0.05, ** p<0.01, *** p<0.001, **** p<0.0001; n.s, non-significant.

FIGS. 3A-3B. PI CART-cells have an enriched population of less differentiated T-cells. See FIG. 2B for cell generation procedure. FIG. 3A. Representative flow plots showing memory phenotypes of specified T-cell types as determined by CCR7 and CD45RA staining. FIG. 3B. Summary of percentages of CCR7⁺CD45RA⁺ naive-like T-cells (T_{N}), CCR7⁺CD45RA⁻central memory T-cells (T_{CM}), CCR7⁻ CD45RA—effector memory T-cells (T_{EM}), and CCR7CD45RN⁺ effector T-cells (T_{E}) within CD4⁺ (left) and CD8⁺ (right) specified T-cell types on day 7 post transduction (mean±SD, n=10).

FIGS. 4A-4B. Short-term cytotoxicity and proliferation of CAR T-cells during cocultures with T-ALL cell lines. See also FIGS. 2E, 2F. FIG. 4A. Cytotoxicity of specified effector T-cells against Jurkat (left, mean±SD, n=10) or CCRF-CEM (right, mean±SD, n=6) target cells at 24 hours after coculture setup. FIG. 4B. Expansion of specified effector T-cells when cocultured with Jurkat (left, mean±SD, n=10) or CCRF-CEM (right, mean±SD, n=6) target cells for 24 hours. Statistical differences are calculated by One-way ANOVA with Tukey's multiple comparisons. ** p<0.01, *** p<0.001, **** p<0.0001; n.s, non-significant.

FIG. 5. Composition of CD7− T-cells in peripheral blood of healthy donors. Frequency of CD7-negative CD4⁺ and CD8⁺T-cells measured by flow cytometry in PBMC collected from healthy donors (mean±SD, n=5).

FIGS. 6A-6L. PI CAR T-cells showed superior anti-tumor activity and long-term persistence in vivo. FIG. 6A. Schematic of model setup for FIGS. 6B-6D. FIG. 6B. Representative IVIS images showing tumor bioluminescence. FIG. 6C. Change of tumor bioluminescence over time in mice receiving the specified T-cell treatment, as measured by IVIS imaging. Each line represents data from one individual animal. FIG. 6D. Animal survival overtime. FIG. 6E. Schematic of model setup for FIGS. 6F-6G. FIG. 6F. Representative IVIS images showing bioluminescence from infused T-cells. FIG. 6G. Change of T-cell bioluminescence overtime in mice receiving the specified T-cell treatment, as measured by IVIS imaging. Each line represents data from one individual animal. FIG. 6H. Schematic of model setup for FIGS. 6I-6L. FIG. 6I. Representative IVIS images showing bioluminescence from infused T-cells. FIG. 6J. Change of T-cell bioluminescence overtime in mice receiving the specified T-cell treatment, as measured by IVIS imaging. Each line represents data from one individual animal. FIG. 6K. Absolute counts of infused CCRF-CEM tumor cells (left) and T-cells (right) in 50 μL peripheral blood on day 15 post T-cell infusion (mean±SD, n=5 for NT, n=6 for CD7 KO, n=6 for PI). FIG. 6L. Animal survival overtime. Statistical differences are calculated by Log-rank test (FIGS. 6D, 6L) or One-way ANOVA with Tukey's multiple comparisons (FIG. 6K). * p<0.05, ** p<0.01, *** p<0.001, ****p<0.0001.

FIGS. 7A-7B. PI CAR T-cells generated from different donors persisted long-term in the Jurkat xenograft model. See also FIGS. 6E-6G. FIG. 7A. Schematic of model setup for FIG. 7B. FIG. 7B. Change of PI CAR T-cell bioluminescence over time, as measured by IVIS imaging. Each line represents data from one individual animal.

FIGS. 8A-8G. Persisting PI CAR T-cells lack CD7 expression and transcriptionally resemble CD7-edited CAR T-cells. See FIG. 6E and FIG. 5A for model setup. FIG. 8A. Representative flow plots (left) and summary of percent of CAR+CD7− cells (right) pre- and at 27 days post-infusion (mean±SD, n=3 for pre-infusion, n=13 for post-infusion). FIG. 8B. CD7 protein expression in persisting Ctrl non-transduced T-cells and PI CAR T-cells. FIG. 8C. Quantitative PCR results showing relative CD7 mRNA level of post-infusion PI CAR T-cells compared to pre-infusion (mean±SD, n=3 for pre-infusion, n=6 for post-infusion). FIG. 8D. Percent of CD4+ and CD8+ cells in the starting cell material (PBMC) and persisting CD7− PI CAR T-cells post-infusion (mean±SD, n=5 for PBMC, n=13 for post-infusion). FIG. 8E. Heatmap was plotted using normalized gene expression from each sample. Gene expression was normalized with Trimmed Means of M values (TMM) and log 2 transformed counts per million (log 2 (CPM)). Results of unsupervised clustering are shown. FIG. 8F. Scatterplot showing significantly high transcriptome profiling correlation between CD7-unedited and edited CD7 CAR T-cells. Mean normalized gene expression was calculated by averaging normalized gene expression (same method as described in FIG. 8E) from three biological replicates in each condition. P-value and coefficient was calculated with linear regression. Highlighted are genes involved in regulating the immune function of T-cells. Statistical differences are calculated by unpaired two-tailed t test (FIGS. 8A, 8C). **** p<0.0001. FIG. 8G. Jurkat cells (HLA-A2-negative) and donor T-cells (HLA-A2-positive) were analyzed by flow cytometry in peripheral blood on day 32 post T-cell injection. Expression of CD7 and CD7 CAR on tumor cells (left, NT Ctrl group), CD7 KO CD7 CAR T-cells (center) and unedited PI CD7 CAR T-cells (right).

FIGS. 9A-9F. Characterization of cGMP-manufactured autologous PI CAR T-cells for T-ALL patients. FIG. 9A. Frequency of CD7-negative normal CD4+ and CD8+ T-cells measured by flow cytometry in PBMC collected from patients with T-cell malignancies. FIG. 9B. Left: Absolute T-cell counts for each patient at the time of transduction and before cryopreservation 4 days later. Right: Fold expansion of PI CAR T-cells between transduction and cryopreservation. FIG. 9C. Viability of PI CAR T-cells at the time of cryopreservation. FIG. 9D. Percent of CAR+ T-cells at the time of cryopreservation. FIG. 9E. Vector copy number per transduced T-cell at the time of cryopreservation. FIG. 9F. Cytotoxicity of PI CAR T-cells upon coculture with FFluc-labeled Jurkat T-ALL cell line for 24 hours. In all panels, each dot represents data from an individual patient. Mean±SD are shown.

FIG. 10. Cytotoxicity of CD2 CAR T-cells expanded in the presence of dasatinib and ibrutinib against a CD2+ T-cell line. FIG. 10A. Viability and CAR expression in CD2 CAR-transduced T-cells expanded with vehicle control or dasatinib+ibrutinib. NT, non-transduced T cells. FIG. 10B. Cytotoxicity of CD2 CAR T-cells expanded with dasatinib and ibrutinib against a CD2+ cell line Jurkat. Residual counts of live tumor cells were enumerated by flow cytometry at indicated time points.

FIGS. 11A-11C. Illustration of concept of self-terminating CAR T-cells. Target antigen A can be normally expressed in T-cells (e.g., CD5 on CD5 CAR-T, CD7 on CD7 CAR-T, CD2 on CD2 CAR-T) or artificially overexpressed (e.g., CD19 on CD19 CAR-T).

### DETAILED DESCRIPTION

The present disclosure fulfills certain needs in the fields of cell biology, molecular biology, immunology, and medicine, including cancer medicine, by providing compositions and methods directed to therapies for diseases including but not limited to cancer, immune disorders, and infectious diseases caused by infectious disease microorganisms, particularly utilizing adoptive cell therapy that targets disease-associated antigens, for example, cancer cell antigens, immune cell antigens, and infectious disease microorganism antigens, for treatment and prevention of diseases including but not limited to cancer, immune disorders, and infectious diseases caused by infectious disease microorganisms and is based, at least in part, on the surprising discovery that functional cytotoxic genetically engineered immune cells can be generated for adoptive cell therapy without the use of additional cell engineering strategies to reduce immune cell activation, differentiation, and/or fratricide of the genetically engineered immune cells; prevent impairment of genetically engineered immune cell expansion in culture; and/or prevent rapid exhaustion of the genetically engineered immune cells during expansion of the cells in culture. In particular embodiments, cytotoxic genetically engineered mammalian immune cells of any kind (including at least human T-cells and natural killer (NK) cells) are generated to target antigen(s). The disclosure also encompasses a genetically engineered receptor of any kind (including a chimeric antigen receptor (CAR) or a T-cell receptor (TCR)) that is directed against the target antigen(s).

Developing new engineered adoptive cell therapies for diseases like cancers including hematologic malignancies and solid tumors, immune disorders, and infectious diseases sometimes requires targeting antigens that are also expressed by the genetically engineered immune cells comprising the engineered adoptive cell therapies. This often leads to self-targeting (or fratricide) of the cytotoxic genetically engineered immune cells during expansion of the cells in culture. Fratricide of CAR T-cells targeting T-lineage antigens is a common phenomenon. For example, expression of CARs specific to antigens expressed on T-cells, like CD3ε, TCRβ, CD7, CD38, and NKG2D ligands, can produce strong internecine cytolysis that impairs T-cell expansion^{2-4,11-14}. Continuous ligand-driven CAR signaling also accelerates terminal T-cell differentiation that limits the therapeutic potency of these cells^{15,16}.

Additional modifications are often needed to limit fratricide of cells and allow for efficient expansion, and multiple approaches have been proposed to mitigate this unwanted activity. Such modifications include target gene editing (for example, deletion of the antigen genes in the cytotoxic genetically engineered immune cells) or use of special protein expression blocker (PEBL) receptors that anchor the target antigens inside the endoplasmic reticulum of the cytotoxic genetically engineered immune cells. Genetic disruption of the target antigen on T-cells prior to CAR transduction is one common method, and the inventors previously reported that knocking out the CD7 gene with CRISPR/Cas9 enabled the generation of functional CD7 CAR T-cells with no detectable fratricide^{2,17}. This approach can be combined with TCR gene editing to create non-alloreactive CD7 CAR T-cells suitable for off-the-shelf use⁴. Similarly, genetic disruption of the CD3ε gene reduces fratricide of CD3 CAR T-cells¹¹. An alternative approach is to disrupt intracellular trafficking of surface antigens by anchoring them in the endoplasmic reticulum using PEBL molecules. Preclinical studies have shown PEBL-mediated intracellular retention of CD7 or CD3ε proteins prevented their surface expression and minimized fratricide of CAR T-cells targeting the respective antigens^{3,18}. Further, inhibition of CAR-antigen binding with a blocking antibody helps reduce T-cell cytolysis driven by an NKG2D-based CAR, which recognizes multiple ligands on T-cells¹⁴. All these approaches can be utilized to attenuate fratricide and the associated terminal differentiation and functional exhaustion in T-cells but require additional genetic manipulations and/or costly reagents. For example, genome editing involves additional risks of off-target activity and more complex manufacturing to meet current good manufacturing practice (cGMP) standards, whereas the efficacy of PEBL-mediated trapping heavily depends on the stoichiometry between the PEBL receptor and the target protein and may not be effective against all targets.

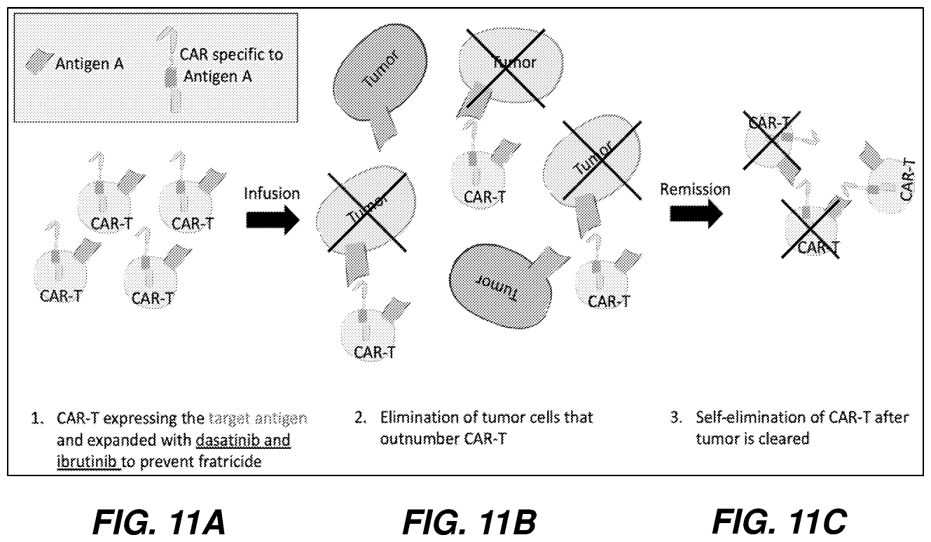

Provided herein and as illustrated by FIG. 11 are alternative methods and compositions directed to minimizing self-targeting of immune cells expressing fratricidal antigen receptors without the need for additional engineering, which can beneficially streamline T-cell manufacturing and reduce its complexity and costs. The method relies on the reversible pharmacological blockade of signaling by genetically engineered receptor(s) using a range of drugs, including FDA-approved tyrosine kinase inhibitors (FIG. 11A). In some embodiments, expanding genetically engineered immune cells in culture in the presence of these compounds minimizes self-directed killing by inhibiting signaling by the genetically engineered receptor. In some embodiments, the cytotoxicity of engineered immune cells is fully regained upon removal of the inhibitors, for example, after administration to a subject in need thereof. Initially, the cells primarily target diseased cells that vastly outnumber the engineered immune cells (FIG. 11B). When the diseased cells are substantially eliminated, the engineered immune cells are more likely to encounter and eliminate one another, thus increasing self-targeting and ultimately regulating expansion, persistence, and activity of the genetically engineered immune cells in vivo (FIG. 11C).

Thus, the disclosure provides cell therapy methods and compositions in which the genetically engineered immune cell therapy is cytotoxic to cells in need of being killed, such as cancer cells, immune cells affected by immune disorders, and/or cells infected by an infectious disease microorganism. The genetically engineered immune cells are generated using a pharmacological blockade mechanism to inhibit signaling by the genetically engineered immune cells when the cytotoxicity of the cells should be deterred. In particular embodiments, the pharmacological blockade mechanism is used to inhibit signaling when the genetically engineered immune cells will kill cells that are not their intended target, such as cells that are not desired to be killed.

In specific embodiments, the cells that are not their intended target are non-diseased, for example, non-cancerous cells, uninfected cells, and/or cells unaffected by an immune disorder.

In specific embodiments, the cells that are not their intended target express one or more target antigens comprising one or more endogenous gene products of the cells that are recognized by the one or more genetically engineered receptors of the genetically engineered immune cells. In some cases, genetically engineered immune cells of the cell therapy express one or more target antigens comprising one or more endogenous gene products of the cells that are recognized by the one or more genetically engineered receptors of the genetically engineered immune cells, which earmarks those cells for destruction by fellow cells of the genetically engineered immune cell therapy.

In specific embodiments, the cells that are not their intended target have acquired through trogocytosis an antigen that otherwise would not have been expressed by the cells, at least to a detectable extent. In some cases, cells of the cell therapy have acquired an antigen through trogocytosis that earmarks those cells for destruction by other cells of the cell therapy, which may or may not also have acquired the antigen through trogocytosis. Trogocytosis is an active cellular process that involves the transfer of surface material from one cell to another, mediated by a constitutive ligand-induced and receptor-mediated antigen endocytosis and recycling process. CAR-mediated trogocytosis has been reported to suppress CAR-T-cell anti-tumor cytotoxicity by mediating fratricide and exhaustion.

In specific embodiments, the cells are self-terminating genetically engineered immune cells that are manipulated to express one or more target antigens recognized by the one or more genetically engineered receptors of the genetically engineered immune cells. In some cases, genetically engineered immune cells of the cell therapy express one or more target antigens that are recognized by the one or more genetically engineered receptors of the genetically engineered immune cells, which earmarks those cells for destruction by fellow cells of the genetically engineered immune cell therapy.

In specific embodiments, one or more target antigens are expressed by only a subset of cells in a population of genetically engineered immune cells. In this case, pharmacological blockade of signaling by the genetically engineered immune cells would limit fratricide of the population subset, preserve the resting state of immune cells that lack the receptor by preventing their activation against antigen-positive cells, and would enable selection of the antigen-negative immune population upon infusion. An example of this is CD7, which is expressed on most but not all T-cells. Expansion of CD7 CAR T-cells with TKIs preserves both CD7+ and CD7-negative populations, but in vivo, only CD7-negative cells are protected from fratricide and persist, producing sustained anti-tumor activity.

The disclosure provides methods and compositions that reduce immune cell activation, differentiation, and/or fratricide among cells of the cell therapy by use of this pharmacological blockade mechanism.

### I. Antigens & Antigen-Targeting

Among the antigens targeted by the genetically engineered receptors disclosed herein are those expressed in the context of any disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas, as well as solid tumors. Also included are immune disorders, such as graft versus host disease, type 1 diabetes, multiple sclerosis, rheumatoid arthritis, psoriatic arthritis, systemic lupus erythematosus, inflammatory bowel disease, Guillain-Barre syndrome, chronic inflammatory demyelinating polyneuropathy, psoriasis, Graves' disease, Hashimoto's thyroiditis, myasthenia gravis, and vasculitis. Also included are infections caused by infectious disease microorganisms. In some embodiments, the target antigen is selectively expressed or overexpressed on cells of the disease or condition, e.g., the tumor, immune, or pathogenic cells, as compared to normal or non-targeted cells or tissues. In other embodiments, the target antigen is expressed on normal cells and/or is expressed on the engineered cells.

The disclosure demonstrates use of antigen-targeting receptors to prevent recognition and killing of certain antigen-expressing cells, and one can utilize this approach when targeting antigens shared between healthy and diseased cells and/or sibling antigen-expressing cells.

Embodiments of the disclosure include use of any of the engineered immune cells encompassed herein. Methods include enhancing cell therapies, including adoptive cell therapies, for individuals in need, such as individuals that have a disease, such as cancer, an immune disorder, or an infection, and for which the engineered immune effectors cells are to be used for therapy. In specific embodiments, the cell therapies employ antigen-targeting receptors that target one or more antigens present on the diseased cells. In specific cases, the genetically engineered immune cells are generated using a pharmacological blockade mechanism to inhibit signaling by the genetically engineered immune cells when the cytotoxicity of the cells should be deterred. In particular embodiments, the pharmacological blockade mechanism is used to inhibit signaling when the genetically engineered immune cells will kill cells that are not their intended target, such as cells that are not desired to be killed.

In specific embodiments, the cells that are not their intended target are non-diseased, for example, non-cancerous cells, uninfected cells, and/or cells unaffected by an immune disorder.

In specific embodiments, the cells that are not their intended target endogenously express the target antigen recognized and bound by one or more antigen-targeting receptors of the genetically engineered immune cells. In some cases, genetically engineered immune cells of the cell therapy express antigen(s) endogenous to the genetically engineered immune cells that are recognized by the one or more antigen-targeting receptors of the genetically engineered immune cells, which earmarks those cells for destruction by fellow cells of the genetically engineered immune cell therapy. In some embodiments, signaling by the one or more antigen-targeting receptors upon binding of the target antigen(s) expressed by the genetically engineered immune cells by one or more antigen-targeting receptors of the genetically engineered immune cells can be reduced upon culture of immune cells manipulated to express the one or more antigen-targeting receptors and/or the genetically engineered immune cells in the presence of one or more tyrosine kinase inhibitors (TKIs). In some cases, a reduction in signaling by the one or more antigen-targeting receptors upon binding of the target antigen(s) expressed by the genetically engineered immune cells reduces immune cell activation, differentiation, and/or fratricide by the genetically engineered immune cells compared to genetically engineered immune cells cultured in the absence of the one or more TKIs.

In specific embodiments, the cells that are not their intended target have acquired through trogocytosis a target antigen that otherwise would not have been expressed by the cells, at least to a detectable extent. In some cases, cells of the cell therapy have acquired a target antigen through trogocytosis that earmarks those cells for destruction by other cells of the cell therapy, which may or may not also have acquired the antigen through trogocytosis. Trogocytosis is an active cellular process that involves the transfer of surface material from one cell to another, mediated by a constitutive ligand-induced and receptor-mediated antigen endocytosis and recycling process. CAR-mediated trogocytosis has been reported to suppress CAR-T-cell anti-tumor cytotoxicity by mediating fratricide and exhaustion. In some embodiments, signaling by the one or more antigen-targeting receptors upon binding of the target antigen(s) acquired via trogocytosis and expressed by the genetically engineered immune cells by one or more antigen-targeting receptors of the genetically engineered immune cells can be reduced upon culture of immune cells manipulated to express the one or more antigen-targeting receptors and/or the genetically engineered immune cells in the presence of one or more tyrosine kinase inhibitors (TKIs). In some cases, a reduction in signaling by the one or more antigen-targeting receptors upon binding of the target antigen(s) acquired via trogocytosis and expressed by the genetically engineered immune cells by the one or more antigen-targeting receptors of the genetically engineered immune cells reduces immune cell activation, differentiation, and/or fratricide by the genetically engineered immune cells compared to genetically engineered immune cells cultured in the absence of the one or more TKIs.

In some cases, the fratricidal activity of the population of genetically engineered immune cells can be restored in vivo after substantial elimination of target cells by the genetically engineered immune cells. In some cases, restoration of the fratricidal activity of the population of genetically engineered immune cells results in elimination of the genetically engineered immune cells upon binding of the target antigen(s) expressed by the genetically engineered immune cells by one or more antigen-targeting receptors also expressed by the genetically engineered immune cells.

In some cases, one or more target antigens recognized by the antigen-targeting receptors of the genetically engineered immune cells are any fratricidal antigen expressed by a cell. In some embodiments, the fratricidal antigens comprise CD1a, CD1b, CD1c, CD1d, CD1e, CD2, CD3d, CD3e, CD3g, CD4, CD5, CD6, CD7, CD8a, CD8b, CD9, CD10, CD11a, CD11b, CD11c, CD11d, CD13, CD14, CD15, CD16a, CD16b, CD17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD60b, CD60c, CD61, CD62E, CD62L, CD62P, CD63, CD64, CD65, CD66a, CD66b, CD66c, CD66d, CD66e, CD66f, CD67, CD68, CD69, CD70, CD71, CD72, CD73, CD74, CD75, CD75s, CD77, CD79a, CD79b, CD80, CD81, CD82, CD83, CD84, CD85a, CD85b, CD85c, CD85d, CD85e, CD85f, CD85g, CD85h, CD85i, CD85j, CD85k, CD86, CD87, CD88, CD89, CD90, CD91, CD92, CD93, CD94, CD95, CD96, CD97, CD98, CD99, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD122, CD123, CD124, CD125, CD126, CD127, CD128, CD129, CD130, CD131, CD132, CD133, CD134, CD135, CD136, CD137, CD138, CD139, CD140a, CD140b, CD141, CD142, CD143, CD144, CD146, CD147, CD148, CD150, CD151, CD152, CD153, CD154, CD155, CD156a, CD156b, CD156c, CD157, CD158a, CD158b1, CD158b2, CD158c, CD158d, CD158e, CD158f1, CD158f2, CD158g, CD158h, CD158i, CD158j, CD158k, CD1587, CD159a, CD159c, CD160, CD161, CD162, CD163, CD163b, CD164, CD165, CD166, CD167a, CD167b, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203a, CD203c, CD204, CD205, CD206, CD207, CD208, CD209, CD210, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD238, CD239, CD240CE, CD240D, CD241, CD242, CD243, CD244, CD245, CD246, CD247, CD248, CD249, CD252, CD253, CD254, CD256, CD257, CD258, CD261, CD262, CD263, CD265, CD266, CD267, CD268, CD269, CD270, CD271, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300b, CD300c, CD300d, CD300e, CD300f, CD300g, CD301, CD302, CD303, CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD360, CD361, CD362, or CD363.

In some cases, one or more target antigens recognized by the antigen-targeting receptors of the genetically engineered immune cells are immune cell lineage antigens. In some embodiments, the immune cell lineage target antigen comprises CD2, CD5, CD7, CD4, CD8, CD3, CS1, CD38, CD99, CD30, 4-1BB, OX40, ICOS, CD26, CD6, TIGIT, PD-1, 2B4, LAG-3, MHC-I, MHC-II, peptide-MHC I, peptide-MHC II, Tim3, CTLA-4, CD112R, CD226, CD96, CD80, CD86, CD112, CD155, KIR2, KIR3, LILRB, CD28, CD40L, CD40, BTLA, GITR, VISTA, NKG2D ligands, or CD70. In some embodiments, the immune cell lineage target antigen comprises CD2. In some embodiments, the immune cell lineage target antigen comprises CD5. In some embodiments, the immune cell lineage target antigen comprises CD7. In some embodiments, the immune cell lineage target antigen comprises CD38.

In some cases, one or more target antigens recognized by the antigen-targeting receptors of the genetically engineered immune cells are antigens acquired via trogocytosis and expressed by the genetically engineered immune cells. The target antigen may be associated with certain cancer cells, infected cells, and/or cells affected by the immune disorder but not associated with non-cancerous cells, non-infected cells, and/or cells unaffected by the immune disorder in some cases. The target antigen may be associated with both certain cancer cells and non-cancerous cells, certain infected cells and non-infected cells, and certain cells affected by the immune disorder and cells unaffected by the immune disorder, in some cases.

In some cases, one or more target antigens recognized by the antigen-targeting receptors of the genetically engineered immune cells are expressed by only a subset of immune cells in a population of genetically engineered immune cells.

Exemplary target antigens include, but are not limited to, antigenic molecules from infectious agents, auto-/self-antigens, tumor-/cancer-associated antigens, and tumor neoantigens (Linnemann et al., 2015). In particular aspects, the antigens include EBNA, CD123, HER1, HER2, CA-125, CA 19-9, CA 72-4, CA 15-3\CA 27.29\BCAA, CA-195, CA-242, CA-50, CA LX, MN-CA IX, TRAIL/DR4, CD2, CD5, CD7, CD19, CD20, CD22, CD23, CD24, CD30, CD33, CD38, CD44v6, CD47, CD56, CD68/P1, CD70, CD97, CD99, CD123, CD171, CD179, CD200, CD319 (CS1), HLA-G, carcinoembryonic antigen, alphafetoprotein, b-human chorionic gonadotropin, AKT, Her3, epithelial tumor antigen, ROR1, folate binding protein, folate receptor, HIV-1 envelope glycoprotein gp120, HIV-1 envelope glycoprotein gp41, HERV-K, Il-6, IL-11Rα, IL-13Rα, kappa chain, lambda chain, CSPG4, CLL-1, U5snRNP200, BAFF-R, BCMA, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, glioma-associated antigen, melanoma-associated antigen, BAGE, DAM-6, DAM-10, GAGE-1, GAGE-2, GAGE-8, GAGE-3, GAGE-4, GAGE-5, GAGE-6, GAGE-7B, pi 5, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, ICE, MUC1, MUC2, MUC16, MUC18, Phosphoinositide 3-kinases (PI3Ks), TRK receptors, PRAME, P15, P16, RU1, RU2, SART-1, SART-3, Wilms' tumor antigen (WT1), AFP, β-catenin, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, E2A-PRL, H4-RET, IGH-IGK, MYL-RAR, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), NY ESO 1, p185erbB2, p180erbB-3, c-Met, nm-23H1, beta-HCG, BCA225, BTAA, CAM 17.1, CAM43, L1 CAM, NCAM, NuMa, 43-9F, 791 Tgp72, CO-029, FGF-5, HTgp-175, M344, MA-50, MG7-Ag, MOV18, NB/70K, NY-CO-1, RCASI, SDCCAG1 6, TA-90\Mac-2 binding protein\cyclophilm C-associated protein, TAAL6, TAG72, TLP, TPS, GPC3, EBMA-1, BARF-1, CS-1, ADRB3, thyroglobulin, EVT6-AML, TGS5, plysialic acid, neutrophil elastase, intestinal carboxyl esterase, prostase, prostein, lewisY, LY6K, PAP, OR51 E2, PANX3, SSEA-4, TARP, CXORF61, Flt3, TEM1, TEM7R, TSHR, UPK2, mammalian targets of rapamycin (mTOR), WNT, extracellular signal-regulated kinases (ERKs), and their regulatory subunits, k-ras, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, EphnnB2, ML-IAP, EpCAM, ERG (TMPRSS2 ETS fusion gene), NA17, PAX3, ALK, androgen receptor, insulin growth factor (IGF)-I, IGFII, IGF-I receptor, cyclin B1, polysialic acid, M-CSF, MYCN, RhoC, GD3, fucosyl GM1, mesothelian, PSCA, sLe, PLAC1, GM3, GPRC5D, GPR20, BORIS, Tn, GLoboH, NY-BR-1, RGsS, SAGE, SART3, STn, PAX5, OY-TES1, sperm protein 17, LCK, HMWMAA, HAVCR1, AKAP-4, SSX2, XAGE 1, B7H3, B7H6, Kit, legumain, TN Ag, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, TSP-180, and LRRN1. Examples of sequences for antigens are known in the art, for example, in the GENBANK® database: CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRVIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, liver, brain, bone, stomach, spleen, testicular, cervical, anal, gall bladder, thyroid, or melanoma cancers, as examples. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostate-carcinoma tumor antigen-1 (PCTA-1), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Antigens may also include genes expressed normally by effector immune cells at various stages of development or functional activation of the effector immune cells, including but not limited to ICOS, 4-1BB, OX40, CD30, CS-1, CD69, CD25, and other typical immune cell markers.

Antigens may include epitopic regions or epitopic peptides derived from genes expressed by or mutated in normal or tumor cells or from genes transcribed at different levels in tumor cells compared to normal cells, such as telomerase enzyme, telomerase reverse transcriptase, survivin, mesothelin, mutated ras, bcr/abl rearrangement, Her1, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus proteins LMP1 and LMP2; nonmutated oncofetal proteins with a tumor-selective expression, such as carcinoembryonic antigen and alpha-fetoprotein.

In other embodiments, instead of a human cellular antigen, such as a cancer antigen (tumor antigen), an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, Staphylococcus species including Methicillin-resistant Staphylococcus aureus (MRSA), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gp120, gp41, p17, and p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (H1, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), IE1 and 1E2 (UL123 and UL122), protein products from the cluster of genes from UL128-UL150 (Rykman, et al., 2006), envelope glycoprotein B (gB), gH, gN, and pp150. As would be understood by the skilled person, CMV proteins for use as antigens described herein may be identified in public databases such as GENBANK®, SWISS-PROT®, and TREMBL® (see e.g., Bennekov et al., 2004; Loewendorf et al., 2010; Marschall et al., 2009).

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al., 2008).

Antigens derived from respiratory syncytial virus (RSV) that are contemplated for use herein include any of the eleven proteins encoded by the RSV genome, or antigenic fragments thereof: NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from vesicular stomatitis virus (VSV) that are contemplated for use include any one of the five major proteins encoded by the VSV genome, and antigenic fragments thereof: large protein (L), glycoprotein (G), nucleoprotein (N), phosphoprotein (P), and matrix protein (M) (see, e.g., Rieder et al., 1999).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis β core or surface antigen, a hepatitis C virus E1 or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be bacterial antigens. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., 2007). As would be understood by the skilled person, Staphylococcus proteins for use as antigens may also be identified in other public databases such as GENBANK®, SWISS-PROT®, and TREMBL®.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneumoniae are also known in the art and may be used as an antigen in some embodiments (see, e.g., Zysk et al., 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®. Proteins of particular interest for antigens according to the present disclosure include virulence factors and proteins predicted to be exposed at the surface of the pneumococci (see, e.g., Frolet et al., 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g., S. pyogenes M proteins), group β streptococcus (S. agalactiae) polypeptides, Treponema polypeptides, and Yersinia polypeptides (e.g., Y. pestis F1 and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

### II. Genetically Engineered Receptors

The immune cells of the present disclosure can be genetically engineered to express one or more antigen-targeting receptors (also referred to herein as “antigen-binding receptors” and “antigen receptors”) that target one or more antigens, such as engineered CARs or, alternatively, engineered TCRs, thereby producing genetically engineered immune cells. For example, the immune cells may be immune cells that are modified to express a CAR and/or TCR having specificity for a cancer cell antigen, an immune cell antigen, or an infectious disease antigen. Other CARs and/or TCRs may be expressed by the same cells as the cancer cell antigen, immune cell antigen, or infectious disease antigen receptor-expressing cells, and they may be directed to different antigens.

In some aspects, the immune cells are engineered to express the cancer cell antigen-specific CAR or cancer cell antigen-specific TCR by transient transfection or transduction of the CAR or TCR. In other cases, the immune cells may be immune cells that are modified to express a CAR and/or TCR having specificity for an infectious disease antigen. Other CARs and/or TCRs may be expressed by the same cells as the infectious disease antigen receptor-expressing cells, and they may be directed to different antigens. In some aspects, the immune cells are engineered to express the infectious disease antigen-specific CAR or infectious disease antigen-specific TCR by transient transfection or transduction of the CAR or TCR. In other cases, the immune cells may be immune cells that are modified to express a CAR and/or TCR having specificity for an immune disorder antigen. Other CARs and/or TCRs may be expressed by the same cells as the immune disorder antigen receptor-expressing cells, and they may be directed to different antigens. In some aspects, the immune cells are engineered to express the immune disorder antigen-specific CAR or immune disorder antigen-specific TCR by transient transfection or transduction of the CAR or TCR.

Suitable methods of modification of cells are known in the art. See, for instance, Sambrook and Ausubel, supra. For example, the cells may be transduced to express a CAR or TCR having antigenic specificity for a cancer antigen using transduction techniques described in Heemskerk et al., 2008 and Johnson et al., 2009.

In some embodiments, the cells comprise one or more nucleic acids introduced via genetic engineering that encode one or more antigen-targeting receptors and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

Exemplary antigen receptors, including CARs and recombinant TCRs, as well as methods for engineering and introducing the receptors into cells, include those described, for example, in international patent application publication numbers WO2000/14257, WO2013/126726, WO2012/129514, WO2014/031687, WO2013/166321, WO2013/071154, WO2013/123061, and WO/2014055668; U.S. patent application publication numbers US2002131960, US2013287748, and US20130149337; U.S. Pat. Nos. 6,451,995, 7,446,190, 8,252,592, 8,339,645, 8,398,282, 7,446,179, 6,410,319, 7,070,995, 7,265,209, 7,354,762, 7,446,190, 7,446,191, 8,324,353, and 8,479,118; and European patent application number EP2537416; those described by Sadelain et al., 2013; Davila et al., 2013; Turtle et al., 2012; Wu et al., 2012; and/or those described by international patent application publication number WO2016138491; U.S. patent application numbers US20200405811, US20190144522, US20200087398, and US20200000937; and U.S. patent number U.S. Pat. No. 10,550,183.

### A. Chimeric Antigen Receptors (CARs)

In particular embodiments, a cancer cell antigen-specific CAR is utilized that comprises at least: a) one or more intracellular signaling domains, b) a transmembrane domain, and c) an extracellular domain comprising at least one antigen binding region that targets, including specifically binds, a cancer cell antigen. In particular embodiments the antigen binding region is an antibody or functional fragment thereof, although in other cases the antigen binding region of the CAR is not an antibody or functional fragment thereof (such as a receptor ligand). In some embodiments, the cancer cell antigen-specific CAR binds a single cancer cell antigen, whereas in other cases the CAR as a single polypeptide is bispecific by comprising two or more antigen binding domains, one of which that binds a first cancer cell antigen and the other of which binds another, non-identical cancer cell antigen.

In particular embodiments, an infectious disease antigen-specific CAR is utilized that comprises at least: a) one or more intracellular signaling domains, b) a transmembrane domain, and c) an extracellular domain comprising at least one antigen binding region that targets, including specifically binds, an infectious disease antigen. In particular embodiments the antigen binding region is an antibody or functional fragment thereof, although in other cases the antigen binding region of the CAR is not an antibody or functional fragment thereof (such as a receptor ligand). In some embodiments, the infectious disease antigen-specific CAR binds a single infectious disease antigen, whereas in other cases the CAR as a single polypeptide is bispecific by comprising two or more antigen binding domains, one of which that binds a first infectious disease antigen and the other of which binds another, non-identical infectious disease antigen.

In particular embodiments, an immune disorder antigen-specific CAR is utilized that comprises at least: a) one or more intracellular signaling domains, b) a transmembrane domain, and c) an extracellular domain comprising at least one antigen binding region that targets, including specifically binds, an immune disorder antigen. In particular embodiments the antigen binding region is an antibody or functional fragment thereof, although in other cases the antigen binding region of the CAR is not an antibody or functional fragment thereof (such as a receptor ligand). In some embodiments, the immune disorder antigen-specific CAR binds an immune disorder antigen, whereas in other cases the CAR as a single polypeptide is bispecific by comprising two or more antigen binding domains, one of which that binds a first immune disorder antigen and the other of which binds another, non-identical immune disorder antigen.

In some embodiments, the genetically engineered antigen receptors include CARs, including activating or stimulatory CARs, or costimulatory CARs (see WO2014/055668). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric CAR construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

Certain embodiments of the present disclosure concern the use of nucleic acids, including nucleic acids encoding a cancer cell antigen-specific CAR polypeptide, including in some cases a CAR that has been humanized to reduce immunogenicity (hCAR), nucleic acids encoding an infectious disease antigen-specific CAR polypeptide, and/or nucleic acids encoding an immune disorder antigen-specific CAR polypeptide, comprising at least one intracellular signaling domain, a transmembrane domain, and an extracellular domain comprising one or more signaling motifs. In certain embodiments, the cancer cell antigen-specific CAR, infectious disease antigen-specific CAR, and/or immune disorder antigen-specific CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In certain embodiments, the antibodies or fragments thereof are the antibodies or fragments thereof are scFv monoclonal antibodies, nanobodies/VHH-only sequences, fibronectin-derived binding domains, DARPINs, or natural ligands. In another embodiment, that specificity is derived from a peptide (e.g., cytokine) that binds to a receptor.

It is contemplated that the human cancer cell antigen CAR nucleic acids may be human genes used to enhance cellular immunotherapy for human patients. In a specific embodiment, the disclosure includes a full-length cancer cell antigen-specific CAR cDNA or coding region. The antigen binding regions or domain can comprise a fragment of the V_{H} and V_{L} chains of a single-chain variable fragment (scFv) derived from a particular human monoclonal antibody. The fragment can also be any number of different antigen binding domains of a human antigen-specific antibody. In a more specific embodiment, the fragment is a cancer cell antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody.

In some embodiments, a cancer cell antigen-specific CAR is constructed with specificity for a particular cancer cell antigen, such as an antigen being expressed on a diseased cell type. Thus, the CAR typically includes in its extracellular portion one or more cancer cell antigen-binding molecules, such as one or more antigen-binding fragments, domains, antibody variable domains, and/or antibody molecules of any kind. Examples of human cancer cell antigen nucleic acids can readily be located in the National Center for Biotechnology Information's GENBANK® database. One of skill in the art is able to generate antibodies, including scFvs against the cancer cell antigens based on knowledge at least of the polypeptide and routine practices, although numerous anti-cancer cell antigen scFvs and monoclonal antibodies are already present in the art.

In some embodiments, an infectious disease antigen-specific CAR is constructed with specificity for a particular infectious disease antigen, such as an antigen being expressed on a diseased cell type. Thus, the CAR typically includes in its extracellular portion one or more infectious disease antigen-binding molecules, such as one or more antigen-binding fragments, domains, antibody variable domains, and/or antibody molecules of any kind. Examples infectious disease cell antigen nucleic acids can readily be located in the National Center for Biotechnology Information's GENBANK® database. One of skill in the art is able to generate antibodies, including scFvs against the infectious disease antigens based on knowledge at least of the polypeptide and routine practices, although numerous anti-infectious disease antigen scFvs and monoclonal antibodies are already present in the art.

In some embodiments, immune disorder antigen-specific CAR is constructed with specificity for a particular immune disorder antigen, such as an antigen being expressed on a diseased cell type. Thus, the CAR typically includes in its extracellular portion one or more immune disorder antigen-binding molecules, such as one or more antigen-binding fragments, domains, antibody variable domains, and/or antibody molecules of any kind. Examples of human immune disorder antigen nucleic acids can readily be located in the National Center for Biotechnology Information's GENBANK® database. One of skill in the art is able to generate antibodies, including scFvs against the immune disorder antigens based on knowledge at least of the polypeptide and routine practices, although numerous anti-immune disorder antigen scFvs and monoclonal antibodies are already present in the art.

In some embodiments, the cancer cell, infectious disease, and/or immune disorder antigen-specific CAR includes an antigen-binding portion or portions of an antibody molecule, such as a single-chain antibody fragment (scFv) derived from the variable heavy (V_{H}) and variable light (V_{L}) chains of a monoclonal antibody (mAb). In specific embodiments, the antibody or functional fragment thereof is or is derived from one or more commercially available antibodies including but not limited to anti-CD5 clones H65, UCHT2, L17F12, CD5-5D7, OTI10H3, OTI2G8, OTI3A9, OTI5D4, CRISI, M28623, OTI2D8, OTI6F7, OTI9E9, OTI10C8, OTI10F4, OTI10H4, OTI12C10, OTI12E10, OTI13C3, OTI13F2, OTIIAS, OTIIA8, OTIIB7, OTIIF9, OTI2A2, OTI2B8, OTI2C2, OTI2E1, OTI3E5, OTI3H4, OTI4A10, OTI4F9, OTI4H3, OTI5F8, OTI5G10, OTISH10, OTI6C9, OTI6D6, OTI7A7, OTI8C10, OTI8E7, UMAB9, 4C7, 6A11, ICO-80, MEM-32, SP19, and the like; anti-CD7 clones 3A1e, 3A1f, TH-69, 124-1D1, 4H9, CD7-6B7, MEM-186, MG34, OTIIA6, 1B8, 1G10D8, 2A4E6, 2D7D11, LT7, and the like; or anti-CD2 clones TS2/18, RPA-2.10, AB75, UMAB6, S5.5, UMAB86, OTI9DI, OTI3E11, OTIIC5, 3A10B2, OTI4E4, OTI2C3, OTISA1, 118, LT2, OTIID4, 224, T6.3, MEM-65, and the like. The antibody may also be one that is generated de novo against the cancer cell, infectious disease, and/or immune disorder antigen, and the scFv sequence may be obtained, or derived, from such de novo antibodies.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof, as it is found that introns stabilize the mRNA. Also, it may be further advantageous to use endogenous or exogenous non-coding regions to stabilize the mRNA.

A hinge portion may link the antigen-binding domain to the transmembrane domain. It should be flexible enough to allow the antigen-binding domain to orient in different directions to facilitate antigen binding. The hinge may be any suitable hinge and includes a hinge derived from IgG, or CD4, CD8, or CD28, in some cases. The hinge portion can comprise an amino acid sequence of a human IgG1, IgG2, IgG3, or IgG4 hinge region. The hinge portion may also include one or more amino acid substitutions and/or insertions and/or deletions compared to a wild-type (naturally-occurring) hinge region. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the C_{H}2 or C_{H}3 domain from human immunoglobulin. One could also use the hinge, C_{H}2 and C_{H}3 region of a human immunoglobulin that has been modified to improve dimerization. One could also use just the hinge portion of an immunoglobulin.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex. The transmembrane domain in some embodiments is derived either from a natural or from a synthetic source. Where the source is natural, the domain in some aspects is derived from any membrane-bound or transmembrane protein. Transmembrane regions include those derived from (i.e., comprise at least the transmembrane region(s) of) the alpha, beta or zeta chain of the T-cell receptor, CD28, DAP12, DAP10, NKG2D, CD35, CD3 epsilon, CD3 gamma, CD3 delta, CD45, CD4, CD5, CD8, CD9, CD16, CD22, CD33, CD37, CD64, CD80, CD86, CD134, CD137, CD154, ICOS/CD278, GITR/CD357, and so forth. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. Optionally, a short oligo- or polypeptide linker, e.g., between 2 and about 10 amino acids in length may form the linkage between the transmembrane domain and the cytoplasmic signaling domain of the CAR. In certain embodiments a glycine-serine doublet provides a particularly suitable linker.

In some embodiments, the cancer cell, infectious disease, and/or immune disorder antigen CAR nucleic acid comprises a sequence encoding other costimulatory receptors, such as a transmembrane domain and one or more intracellular signaling domains. Primary T-cell activation signals, such as may be initiated by CD3ξ and/or FcεRIγ, is responsible for activation of at least one of the normal effector functions of the immune cell in which the CAR has been placed. After antigen and/or ligand recognition, receptors cluster and a signal is transmitted to the cell through the cytoplasmic region. The term “effector function” refers to a specialized function of a cell. An effector function of a T-cell, for example, may be cytolytic activity, or helper activity including the secretion of cytokines. Thus the term “intracellular signaling domain” refers to the portion of a protein that transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion can be used in place of the intact chain as long as it transduces the effector function signal. The term intracellular signaling domain is thus meant to include any truncated portion of the intracellular signaling domain sufficient to transduce the effector function signal.

In addition to a primary T-cell activation signal, such as may be initiated by CD3ξ and/or FcεRIγ, an additional stimulatory signal for immune cell proliferation and effector function following engagement of the chimeric receptor with the target antigen may be utilized. For example, part or all of a human costimulatory receptor for enhanced activation of cells may be utilized that could help improve in vivo persistence and improve the therapeutic success of the adoptive immunotherapy. A costimulatory receptor may refer to the cognate binding partner on an immune cell that specifically binds with a costimulatory ligand, thereby mediating a co-stimulatory response by immune cells, such as, but not limited to, proliferation and/or activation. A costimulatory signal may refer to a signal that in combination with a primary signal, leads to immune cell activation, proliferation, and/or upregulation or downregulation of key molecules.

Costimulatory receptors suitable for use in the CARs of the disclosure include any desired intracellular signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some embodiments, the cytoplasmic region includes CD34, CD16, DAP10, DAP12, CD2, CD7, LFA-1 (CD11a/CD18), CD27, CD28, CD30, CD40, 4-1BB (CD137), CD278, 2B4, DNAM-1, OX40, ICOS, HVEM, LIGHT, ICAM-1, BTLA, GITR, NKG2D, and NKG2C type signaling chains, although in specific alternative embodiments any one of these listed may be excluded from use in the CAR.

In certain embodiments, the platform technologies disclosed herein to genetically modify immune cells, such as T-cells, NK cells, myeloid cells, and B-cells, comprise (i) non-viral gene transfer using an electroporation device (e.g., a nucleofector), (ii) CARs that signal through endodomains (e.g., CD28/CD3-¿, CD137/CD3-3, or other combinations), (iii) CARs with variable lengths of extracellular domains connecting the antigen-recognition domain to the cell surface, and, in some cases, (iv) artificial antigen presenting cells (aAPC) derived from K562 to be able to robustly and numerically expand CAR+ immune cells (Singh et al., 2008; Singh et al., 2011).

### 1. Examples of Specific CAR Embodiments

In particular embodiments, specific target antigen CAR molecules are encompassed herein, such as those that target cancer cell, infectious disease, and/or immune cell antigens. In some cases, the target antigen binding domain of the CAR is a scFv, and any scFv that binds to the target antigen and/or ligand that binds the target antigen may be utilized herein. In cases wherein the target antigen scFv is utilized in the extracellular domain of the CAR, the variable heavy chain and the variable light chain for the scFv may be in any order in N-terminal to C-terminal direction. For example, the variable heavy chain may be on the N-terminal side of the variable light chain, or vice versa. The scFv and/or ligand that binds the target antigen in the CAR may or may not be codon optimized. In particular embodiments, a vector encoding the target antigen-specific CAR and also encodes one or more other molecules. For example, a vector may encode a target antigen-specific CAR and also may encode another protein of interest, such as another engineered antigen receptor.

On the same molecule, the target antigen-specific CAR may comprise one or more antigen-specific extracellular domains, a specific hinge, a specific transmembrane domain, one or more specific cytoplasmic or costimulatory domains, and one or more specific activation signals. When more than one antigen-specific extracellular domain is utilized, such as for targeting two different antigens (one of which is the target antigen), there may be a linker between the two antigen-specific extracellular domains.

In particular embodiments of specific CAR molecules, a CAR may utilize DAP10, DAP12, 4-1BB, NKG2D, or other cytoplasmic domains (which may be referred to herein as costimulatory domains). In some cases, CD32 is utilized without any costimulatory domains. In particular embodiments of specific CAR molecules, a CAR may utilize any suitable transmembrane domain, such as from DAP12, DAP10, NKG2D or CD28.

In particular embodiments, there is an expression construct comprising a sequence that encodes a particular target antigen-specific engineered receptor. In particular embodiments, the expression construct comprises a signal peptide, an antigen-specific extracellular domain, a hinge and/or spacer, a transmembrane domain, and one or more cytoplasmic domains. In particular embodiments, the signal peptide, antigen-specific extracellular domain, hinge and/or spacer, transmembrane domain, and one or more cytoplasmic domains comprise the following order from the C-terminus to the N-terminus in the construct: <signal peptide><antigen-specific extracellular domain><hinge/spacer><transmembrane domain><cytoplasmic domain 1><cytoplasmic domain 2>. In particular embodiments, the signal peptide, antigen-specific extracellular domain, hinge and/or spacer, transmembrane domain, and one or more cytoplasmic domains comprise the following order from the N-terminus to the C-terminus in the construct: <signal peptide><antigen-specific extracellular domain ><hinge/spacer><transmembrane domain><cytoplasmic domain 1><cytoplasmic domain 2>.

In particular embodiments, any target-antigen specific CAR may comprise one of the following: an anti-CD7 scFv, an IgG4/IgG1 Fc-derived spacer, a CD28-derived transmembrane domain, and CD28- and CD3ξ-derived cytoplasmic domains; (b) an anti-CD7 scFv, a CD8a-derived spacer, a CD28-derived transmembrane domain, and CD28- and CD3ξ-derived cytoplasmic domains; (c) an anti-CD5 scFv, an IgG4/IgG1 Fc-derived spacer, a CD28-derived transmembrane domain, and CD28- and CD3ξ-derived cytoplasmic domains; (d) an anti-CD5 scFv, a CD8a-derived spacer, a CD28-derived transmembrane domain, and CD28- and CD3ξ-derived cytoplasmic domains; (e) an anti-CD2 scFv, an IgG4/IgG1 Fc-derived spacer, a CD28- derived transmembrane domain, and CD28- and CD3ξ-derived cytoplasmic domains; and (f) an anti-CD2 scFv, a CD8a-derived spacer, a CD28-derived transmembrane domain, and CD28- and CD3ξ-derived cytoplasmic domains.

Examples of specific sequence embodiments are provided below.

a. Signal Peptide

In specific embodiments, a CD8a signal peptide nucleotide sequence is utilized, as follows:

(SEQ ID NO: 1)
ATGGCCTTACCAGTGACCGCCTTGCTCCTGCCGCTGGCCTTGCTGCTCC
ACGCCGCCAGGCCG

An amino acid sequence translated from SEQ ID NO: 1 is as follows:

(SEQ ID NO: 2)
MALPVTALLLPLALLLHAARP

In specific embodiments, an IgV signal peptide nucleotide sequence is utilized, as follows:

(SEQ ID NO: 3)
ATGGAGTTTGGGCTGAGCTGGCTTTTTCTTGTGGCTATTTTAAAAGGTG
TCCAGTGC

An amino acid sequence translated from SEQ ID NO:3 is as follows:

(SEQ ID NO: 4)
MEFGLSWLFLVAILKGVQC

In some embodiments, the signal peptide nucleotide sequence has at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides, or any value derivable therein, and has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any range or value derivable therein, with SEQ ID NO:1 or SEQ ID NO:3. In some embodiments, the signal peptide nucleotide sequence comprises SEQ ID NO:1 or SEQ ID NO:3. In some embodiments, the signal peptide nucleotide sequence consists of SEQ ID NO:1 or SEQ ID NO: 3.

In some embodiments, the signal peptide amino acid sequence has at least 5, 6, 7, 8, 9, 10, 11, 12, 13, 14, 15, 16, 17, 18, 19, 20, 21, 22, 23, 24, 25, 26, 27, 28, 29, or 30 amino acids, or any range or value derivable therein, and has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:2 or SEQ ID NO:4. In some embodiments, the signal peptide amino acid sequence comprises SEQ ID NO:2 or SEQ ID NO:4. In some embodiments, the signal peptide amino acid sequence consists of SEQ ID NO:2 or SEQ ID NO:4.

b. Antigen-Specific Extracellular Domains

In specific embodiments, an anti-CD5 scFv nucleotide sequence is utilized, as follows:

(SEQ ID NO: 5)
ATGGAGTTTGGGCTGAGCTGGCTTTTTCTTGTGGCTATTTTAAAAGGTG
TCCAGTGCATCGATGCCATGGGCAACATCCAGCTGGTGCAGAGCGGCCC
TGAGCTGAAGAAACCCGGCGAGACAGTGAAGATCAGCTGCAAGGCCAGC
GGCTACACCTTCACCAACTACGGCATGAACTGGGTGAAACAGGCCCCAG
GCAAGGGCCTGCGGTGGATGGGCTGGATCAACACCCACACCGGCGAGCC
CACCTACGCCGACGACTTCAAGGGCAGATTCGCCTTCAGCCTGGAAACC
AGCGCCAGCACCGCCTACCTGCAGATCAACAACCTGAAGAACGAGGACA
CCGCCACCTATTTCTGCACCAGACGGGGCTACGACTGGTACTTCGACGT
GTGGGGAGCCGGCACCACCGTGACCGTGTCTAGCGGAGGCGGAGGATCT
GGCGGAGGGGGATCAGGCGGCGGAGGCAGCGACATCAAGATGACCCAGA
GCCCCAGCTCTATGTACGCCAGCCTGGGCGAGCGCGTGACCATCACATG
CAAGGCCTCCCAGGACATCAACAGCTACCTGAGCTGGTTCCACCACAAG
CCCGGCAAGAGCCCCAAGACCCTGATCTACCGGGCCAACCGGCTGGTGG
ACGGCGTGCCAAGCAGATTCAGCGGCAGCGGCTCCGGCCAGGACTACAG
CCTGACCATCAGCAGCCTGGACTACGAGGACATGGGCATCTACTACTGC
CAGCAGTACGACGAGAGCCCCTGGACCTTCGGAGGCGGCACCAAGCTGG
AAATGAAGGGCAGCGGGGATCCCGCC

A translated scFv (translated from SEQ ID NO:5) amino acid sequence is as follows:

(SEQ ID NO: 6)
MEFGLSWLFLVAILKGVQCIDAMGNIQLVQSGPELKKPGETVKISCKAS
GYTFTNYGMNWVKQAPGKGLRWMGWINTHTGEPTYADDFKGRFAFSLET
SASTAYLQINNLKNEDTATYFCTRRGYDWYFDVWGAGTTVTVSSGGGGS
GGGGSGGGGSDIKMTQSPSSMYASLGERVTITCKASQDINSYLSWFHHK
PGKSPKTLIYRANRLVDGVPSRFSGSGSGQDYSLTISSLDYEDMGIYYC
QQYDESPWTFGGGTKLEMKGSGDPA

In specific embodiments, an anti-CD7 scFv nucleotide sequence is utilized, as follows:

(SEQ ID NO: 7)
CAGGTGAAGCTGCAGGAGTCAGGGGGAGGCTTAGTGAAGCCTGGAGG
GTCCCTGAAACTCTCCTGTGCAGCCTCTGGATTCACTTTCAGTAGCTAT
GCaATGTCTTGGGTTCGCCAGACTCCGGAGAAGAGGCTGGAGTGGGTCG
CAACCATTAGTAGTGGTGGTAGTTACACCTACTATCCAGACAGTGTGAA
GGGGCGATTCACCATCTCCAGAGACAATGCCAAGAACACCCTGTACCTG
CAAATGAGCAGTCTGAGGTCTGAGGACACGGCCATGTATTACTGTGCAA
GACAGGATGGTTACTACCCGGGCTGGTTTGCTAACTGGGGGCAAGGGAC
CACGGTCACCGTCTCCTCAGGTGGAGGCGGTTCAGGCGGAGGTGGCTCT
GGCGGTGGCGGATCGGACATCGAGCTCACTCAGTCTCCAGCAATCATGT
CTGCATCTCTAGGGGAGGAGATCACCCTAACCTGCAGTGCCAGCTCcAG
TGTAAGTTACATGCACTGGTACCAGCAGAAGTCAGGCACTTCTCCCAAA
CTCTTGATTTATAGCACATCCAACCTGGCTTCTGGAGTCCCTTCTCGCT
TCAGTGGCAGTGGGTCTGGGACCTTTTATTCTCTCACAATCAGCAGTGT
GGAGGCTGAAGATGCTGCCGATTATTACTGCCATCAGTGGAGTAGTTAC
ACGTTCGGAGGGGGCACCAAGCTGGAAATCAAACGGGCG

A translated scFv (translated from SEQ ID NO:7) amino acid sequence is as follows:

(SEQ ID NO: 8)
PQVKLQESGGGLVKPGGSLKLSCAASGFTFSSYAMSWVRQTPEKRLEWV
ATISSGGSYTYYPDSVKGRFTISRDNAKNTLYLQMSSLRSEDTAMYYCA
RQDGYYPGWFANWGQGTTVTVSSGGGGSGGGGSGGGGSDIELTQSPAIM
SASLGEEITLTCSASSSVSYMHWYQQKSGTSPKLLIYSTSNLASGVPSR
FSGSGSGTFYSLTISSVEAEDAADYYCHQWSSYTFGGGTKLEIKRA

In specific embodiments, an anti-CD7 scFv nucleotide sequence is utilized, as follows:

(SEQ ID NO: 9)
ATGGCCCTGCCTGTGACCGCTCTGCTGCTGCCTCTGGCACTGCTGCTGC
ACGCTGCTAGACCTGGCGCTCAGCCTGCTATGGCCGCCTACAAGGACAT
CCAGATGACCCAGACCACCAGCAGCCTGTCTGCCAGCCTGGGCGACAGA
GTGACCATCAGCTGTAGCGCCAGCCAGGGCATCAGCAACTACCTGAACT
GGTATCAGCAGAAACCCGACGGCACCGTGAAGCTGCTGATCTACTACAC
CAGCTCCCTGCACAGCGGCGTGCCCAGCAGATTTTCTGGCAGCGGCTCC
GGCACCGACTACAGCCTGACCATCTCCAACCTGGAACCCGAGGATATCG
CCACCTACTACTGCCAGCAGTACAGCAAGCTGCCCTACACCTTCGGCGG
AGGCACCAAGCTGGAAATCAAGAGGGGAGGCGGAGGAAGCGGAGGCGGT
GGATCTGGTGGTGGCGGTTCTGGCGGAGGTGGAAGCGAAGTGCAGCTGG
TGGAATCTGGCGGCGGACTGGTCAAGCCTGGCGGCTCTCTGAAACTGAG
CTGTGCCGCCTCTGGCCTGACCTTCAGCAGCTACGCTATGAGCTGGGTG
CGCCAGACCCCCGAGAAGAGACTGGAATGGGTGGCCAGCATCAGCAGCG
GCGGCTTTACCTACTACCCCGACAGCGTGAAGGGCCGGTTCACCATCAG
CCGGGACAACGCCCGGAACATCCTGTACCTGCAGATGAGCAGCCTGCGG
AGCGAGGACACCGCCATGTACTACTGCGCCAGGGATGAAGTGCGGGGCT
ACCTGGATGTGTGGGGAGCCGGAACAACCGTGACCGTGTCTAGTGCCAG
CGGAGCGGATCC

A translated scFv (translated from SEQ ID NO:9) amino acid sequence is as follows:

(SEQ ID NO: 10)
MALPVTALLLPLALLLHAARPGAQPAMAAYKDIQMTQTTSSLSASLGDR
VTISCSASQGISNYLNWYQQKPDGTVKLLIYYTSSLHSGVPSRFSGSGS
GTDYSLTISNLEPEDIATYYCQQYSKLPYTFGGGTKLEIKRGGGGSGGG
GSGGGGSGGGGSEVQLVESGGGLVKPGGSLKLSCAASGLTFSSYAMSWV
RQTPEKRLEWVASISSGGFTYYPDSVKGRFTISRDNARNILYLQMSSLR
SEDTAMYYCARDEVRGYLDVWGAGTTVTVSSASGADPA

In specific embodiments, an anti-CD7 scFv nucleotide sequence is utilized, as follows:

(SEQ ID NO: 11)
ATGGCCTTACCAGTGACCGCCTTGCTCCTGCCGCTGGCCTTGCTGCTCC
ACGCCGCCAGGCCGCAGGTCCAGCTGCAGGAGTCTGGGGCTGAACTGGT
GAAGCCTGGGGCTTCAGTGAAGCTGTCCTGCAAGGCTTCTGGCTACACC
TTCACGAGCTACTGGATGCACTGGGTGAAGCAGAGGCCTGGACAAGGCC
TTGAGTGGATTGGAAAGATTAATCCTAGCAACGGTCGTACTAACTACAA
TGAGAAGTTCAAGAGCAAGGCCACACTGACTGTAGACAAATCCTCCAGC
ACAGCCTACATGCAACTCAGCAGCCTGACATCTGAGGACTCTGCGGTCT
ATTACTGTGCAAGAGGGGGAGTCTACTATGACCTTTATTACTATGCTCT
GGACTACTGGGGCCAAGGCACCACGGTCACCGTCTCCTCAGGTGGAGGC
GGTTCAGGCGGAGGTGGCTCTGGCGGTGGCGGATCGGACATCGAGCTCA
CTCAGTCTCCAGCCACCCTGTCTGTGACTCCAGGAGATAGCGTCAGTCT
TTCCTGCAGGGCCAGCCAAAGTATTAGCAACAACCTACACTGGTATCAA
CAAAAATCACATGAGTCTCCAAGGCTTCTCATCAAGTCTGCTTCCCAGT
CCATCTCTGGaATCCCCTCCAGGTTCAGTGGCAGTGGATCAGGGACAGA
TTTCACTCTCAGTATCAACAGTGTGGAGACTGAAGATTTTGGAATGTAT
TTCTGTCAACAGAGTAACAGCTGGCCGTACACGTTCGGAGGGGGGACAA
AGTTGGAAATAAAACGGGCGGATCC

A translated scFv (translated from SEQ ID NO:11) amino acid sequence is as follows:

(SEQ ID NO: 12)
MALPVTALLLPLALLLHAARPQVQLQESGAELVKPGASVKLSCKASGYT
FTSYWMHWVKQRPGQGLEWIGKINPSNGRTNYNEKFKSKATLTVDKSSS
TAYMQLSSLTSEDSAVYYCARGGVYYDLYYYALDYWGQGTTVTVSSGGG
GSGGGGSGGGGSDIELTQSPATLSVTPGDSVSLSCRASQSISNNLHWYQ
QKSHESPRLLIKSASQSISGIPSRFSGSGSGTDFTLSINSVETEDFGMY
FCQQSNSWPYTFGGGTKLEIKRADPA

In specific embodiments, an anti-CD2 scFv nucleotide sequence is utilized, as follows:

(SEQ ID NO: 13)
GATGTTGTTCTTACTCAGACTCCACCAACTTTGTTGGCAACAATTGGGC
AAAGTGTGTCAATTAGTTGCAGATCAAGCCAAAGTCTCTTGCACAGTAG
CGGAAATACCTATCTGAACTGGCTGTTGCAGCGGACTGGGCAATCCCCG
CAACCGCTCATATACCTGGTAAGCAAGCTAGAGTCAGGGGTGCCGAATC
GCTTCTCCGGATCCGGTAGTGGTACGGATTTCACGCTGAAGATAAGCGG
AGTGGAAGCGGAAGACTTGGGCGTGTACTACTGTATGCAGTTCACACAC
TATCCCTACACTTTTGGGGGGGGTACTAAACTTGAGCTTAAGTCTGGAG
GCGGTGGATCTGGCGGTGGAGGTAGCGGAGGAGGCGGTAGCGAAGTGCA
ATTGCAGCAGTCAGGGCCAGAGCTGCAAAGACCTGGTGCCAGCGTGAAG
TTGTCCTGTAAAGCCTCCGGTTATATCTTCACAGAGTACTATATGTACT
GGGTTAAGCAACGCCCAAAACAAGGCCTGGAGCTTGTGGGCCGAATCGA
CCCCGAAGATGGTTCTATTGACTACGTAGAGAAGTTCAAGAAAAAGGCA
ACACTCACTGCGGACACTAGTTCAAACACTGCCTACATGCAGCTCTCTA
GCCTGACATCCGAAGACACCGCCACGTATTTTTGCGCACGAGGTAAATT
CAACTATCGCTTCGCATACTGGGGGCAGGGTACTCTCGTCACCGTCTCC
TCA

A translated scFv (translated from SEQ ID NO:13) amino acid sequence is as follows:

(SEQ ID NO: 14)
DVVLTQTPPTLLATIGQSVSISCRSSQSLLHSSGNTYLNWLLQRTGQSP
QPLIYLVSKLESGVPNRFSGSGSGTDFTLKISGVEAEDLGVYYCMQFTH
YPYTFGAGTKLELKSGGGGSGGGGSGGGGSEVQLQQSGPELQRPGASVK
LSCKASGYIFTEYYMYWVKQRPKQGLELVGRIDPEDGSIDYVEKFKKKA
TLTADTSSNTAYMQLSSLTSEDTATYFCARGKFNYRFAYWGQGTLVTVS
SA

In specific embodiments, an anti-CD2 scFv nucleotide sequence is utilized, as follows:

(SEQ ID NO: 15)
GAAGTGCAATTGCAGCAGTCAGGGCCAGAGCTGCAAAGACCTGGTGC
CAGCGTGAAGTTGTCCTGTAAAGCCTCCGGTTATATCTTCACAGAGTAC
TATATGTACTGGGTTAAGCAACGCCCAAAACAAGGCCTGGAGCTTGTGG
GCCGAATCGACCCCGAAGATGGTTCTATTGACTACGTAGAGAAGTTCAA
GAAAAAGGCAACACTCACTGCGGACACTAGTTCAAACACTGCCTACATG
CAGCTCTCTAGCCTGACATCCGAAGACACCGCCACGTATTTTTGCGCAC
GAGGTAAATTCAACTATCGCTTCGCATACTGGGGGCAGGGTACTCTCGT
CACCGTCTCCTCATCTGGAGGCGGTGGATCTGGCGGTGGAGGTAGCGGA
GGAGGCGGTAGCGATGTTGTTCTTACTCAGACTCCACCAACTTTGTTGG
CAACAATTGGGCAAAGTGTGTCAATTAGTTGCAGATCAAGCCAAAGTCT
CTTGCACAGTAGCGGAAATACCTATCTGAACTGGCTGTTGCAGCGGACT
GGGCAATCCCCGCAACCGCTCATATACCTGGTAAGCAAGCTaGAGTCAG
GGGTGCCGAATCGCTTCTCCGGATCCGGTAGTGGTACGGATTTCACGCT
GAAGATAAGCGGAGTGGAAGCGGAAGACTTGGGCGTGTACTACTGTATG
CAGTTCACACACTATCCCTACACTTTTGGGGGGGGTACTAAACTTGAGC
TTAAGGCC

A translated scFv (translated from SEQ ID NO:15) amino acid sequence is as follows:

(SEQ ID NO: 16)
EVQLQQSGPELQRPGASVKLSCKASGYIFTEYYMYWVKQRPKQGLELVG
RIDPEDGSIDYVEKFKKKATLTADTSSNTAYMQLSSLTSEDTATYFCAR
GKFNYRFAYWGQGTLVTVSSSGGGGSGGGGSGGGGSDVVLTQTPPTLLA
TIGQSVSISCRSSQSLLHSSGNTYLNWLLQRTGQSPQPLIYLVSKLESG
VPNRFSGSGSGTDFTLKISGVEAEDLGVYYCMQFTHYPYTFGAGTKLEL
KA

In specific embodiments, an anti-CD38 scFv nucleotide sequence is utilized, as follows:

(SEQ ID NO: 17)
GCCCAGCCGGCCATGGCCAAGGTCCAGCTGCAGGAGTCAGGACCTAG
CCTAGTGCAGCCCTCACAGCGCCTGTCCATAACCTGCACAGTCTCTGGT
TTCTCATTAATTAGTTATGGTGTACACTGGGTTCGCCAGTCTCCAGGAA
AGGGTCTGGAGTGGCTGGGAGTGATATGGAGAGGTGGAAGCACAGACTA
CAATGCAGCTTTCATGTCCAGACTGAGCATCACCAAGGACAACTCCAAG
AGCCAAGTTTTCTTTAAAATGAACAGTCTGCAAGCTGATGACACTGCCA
TATACTTCTGTGCCAAAACCTTGATTACGACGGGCTATGCTATGGACTA
CTGGGGCCAAGGGACCACGGTCACCGTCTCCTCAGGTGGAGGCGGTTCA
GGCGGAGGTGGCTCTGGCGGTGGCGGATCGGACATCGAGCTCACTCAGT
CTCCATCCTCCTTTTCTGTATCTCTAGGAGACAGAGTCACCATTACTTG
CAAGGCAAGTGAGGACATATATAATCGGTTAGCCTGGTATCAGCAGAAA
CCAGGAAATGCTCCTAGGCTCTTAATATCTGGTGCAACCAGTTTGGAAA
CTGGGGTTCCTTCAAGATTCAGTGGCAGTGGATCTGGAAAGGATTACAC
TCTCAGCATTACCAGTCTTCAGACTGAAGATGTTGCTACTTATTACTGT
CAACAGTATTGGAGTACTCCTACGTTCGGTGGAGGGACCAAGCTGGAAA
TCAAACGG

A translated scFv (translated from SEQ ID NO:17) amino acid sequence is as follows:

(SEQ ID NO: 18)
AQPAMAKVQLQESGPSLVQPSQRLSITCTVSGFSLISYGVHWVRQSPGK
GLEWLGVIWRGGSTDYNAAFMSRLSITKDNSKSQVFFKMNSLQADDTAI
YFCAKTLITTGYAMDYWGQGTTVTVSSGGGGSGGGGSGGGGSDIELTQS
PSSFSVSLGDRVTITCKASEDIYNRLAWYQQKPGNAPRLLISGATSLET
GVPSRFSGSGSGKDYTLSITSLQTEDVATYYCQQYWSTPTFGGGTKLEI
KR

In some embodiments, the antigen-specific extracellular domain nucleotide sequence has at least 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, or 900 nucleotides, or any range or value derivable therein, and has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:5, 7, 9, 11, 13, 15, or 17. In some embodiments, the antigen-specific extracellular domain nucleotide sequence comprises SEQ ID NO:5, 7, 9, 11, 13, 15, or 17. In some embodiments, the antigen-specific extracellular domain nucleotide sequence consists of SEQ ID NO:5, 7, 9, 11, 13, 15, or 17.

In some embodiments, the antigen-specific extracellular domain amino acid sequence has at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 amino acids, or any range or value derivable therein, and has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:6, 8, 10, 12, 14, 16, or 18. In some embodiments, the antigen-specific extracellular domain amino acid sequence comprises SEQ ID NO:6, 8, 10, 12, 14, 16, or 18. In some embodiments, the antigen-specific extracellular domain amino acid sequence consists of SEQ ID NO:6, 8, 10, 12, 14, 16, or 18.

c. Transmembrane Domains

Any suitable transmembrane domain may be utilized in the target antigen-specific CAR. Examples include at least from DAP10, DAP12, CD28, NKG2D, CD3 epsilon, CD3 gamma, CD3 delta, CD4, CD5, CD8, CD9, CD16, CD22, CD28, CD33, CD37, CD45, CD64, CD80, CD86, CD134, CD137, CD154, from a T-cell receptor α or b chain, a CD3} chain, ICOS, GITR/CD357, functional derivatives thereof, and combinations thereof. In specific cases, the transmembrane domain from CD28 is utilized. Examples of particular transmembrane domain sequences that may be used include the following:

CD28 transmembrane domain nucleotide sequence:

(SEQ ID NO: 19)
TTTTGGGTGCTGGTGGTGGTTGGTGGAGTCCTGGCTTGCTATAGCTTGC
TAGTAACAGTGGCCTTTATTATTTTCTGGGTGAGGAGT.

An amino acid sequence translated from SEQ ID NO:19 is as follows:

(SEQ ID NO: 20)
FWVLVVVGGVLACYSLLVTVAFIIFWVRS

In some embodiments, the transmembrane domain nucleotide sequence has at least 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides, or any range or value derivable therein, and has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO: 19. In some embodiments, the transmembrane domain nucleotide sequence comprises SEQ ID NO: 19. In some embodiments, the transmembrane domain nucleotide sequence consists of SEQ ID NO: 19.

In some embodiments, the transmembrane domain amino acid sequence has at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35, or any range or value derivable therein, and has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:20. In some embodiments, the transmembrane domain amino acid sequence comprises SEQ ID NO:20. In some embodiments, the transmembrane domain amino acid sequence consists of SEQ ID NO:20.

d. Cytoplasmic Domains

One or more cytoplasmic domains (that may also be referred to herein as signaling domains or stimulatory domains or costimulatory domains or intracytoplasmic domains, in appropriate cases) may or may not be utilized in specific anti-target antigen CARs of the disclosure. Specific examples include cytoplasmic domains from CD2, CD3ξ, CD3δ, CD3ε, CD3γ, Fc receptors, CD79a, CD79b, CLEC-2, CD7, LFA-1 (CD11a/CD18), CD27, CD28, CD30, CD40, 4-1BB (CD137), CD278, 2B4, DNAM-1, OX40, NKG2C, NKG2D, DAP10, DAP12, B7-1/CD80, CD28, 4-1BBL, B7-2/CD86, CTLA-4, B7-H1/PD-L1, ICOS, B7-H2, PD-1, B7-H3, PD-L2, B7-H4, PDCD6, HVEM, LIGHT, ICAM-1, BTLA, GITR, or a combination thereof. In specific cases, the cytoplasmic domain from CD28, 4-1BB, and/or CD3ξ is utilized. Examples of particular cytoplasmic domain sequences that may be used include the following:

CD28 cytoplasmic domain nucleotide sequence:

(SEQ ID NO: 21)
AAGAGGAGCAGGCTCCTGCACAGTGACTACATGAACATGACTCCCCG
CCGCCCCGGGCCCACCCGCAAGCATTACCAGCCCTATGCCCCACCACGC
GACTTCGCAGCCTATCGCTCC.

An amino acid sequence translated from SEQ ID NO:21 is as follows:

(SEQ ID NO: 22)
KRSRLLHSDYMNMTPRRPGPTRKHYQPYAPPRDFAAYRS

4-1BB cytoplasmic domain amino acid sequence:

(SEQ ID NO: 23)
AAACGGGGCAGAAAGAAACTCCTGTATATATTCAAACAACCATTTATG
AGACCAGTACAAACTACTCAAGAGGAAGATGGCTGTAGCTGCCGATTTC
CAGAAGAAGAAGAAGGAGGATGTGAACTG.

An amino acid sequence translated from SEQ ID NO:23 is as follows:

(SEQ ID NO: 24
KRGRKKLLYIFKQPFMRPVQTTQEEDGCSCRFPEEEEGGCEL

CD3 ξ cytoplasmic domain nucleotide sequence:

(SEQ ID NO: 25)
AGAGTGAAGTTCAGCAGGAGCGCAGACGCCCCCGCGTACCAGCAGGG
CCAGAACCAGCTCTATAACGAGCTCAATCTAGGACGAAGAGAGGAGTAC
GATGTTTTGGACAAGAGACGTGGCCGGGACCCTGAGATGGGGGGAAAGC
CGAGAAGGAAGAACCCTCAGGAAGGCCTGTACAATGAACTGCAGAAAGA
TAAGATGGCGGAGGCCTACAGTGAGATTGGGATGAAAGGCGAGCGCCGG
AGGGGCAAGGGGCACGATGGCCTTTACCAGGGTCTCAGTACAGCCACCA
AGGACACCTACGACGCCCTTCACATGCAGGCCCTGCCCCCTCGC.

An amino acid sequence translated from SEQ ID NO:25 is as follows:

(SEQ ID NO: 26)
RVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTAT
KDTYDALHMQALPPR

In some embodiments, the cytoplasmic domain nucleotide sequence has at least 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, or 350 nucleotides, or any range or value derivable therein, and has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:21, 23, 25. In some embodiments, the cytoplasmic domain nucleotide sequence comprises SEQ ID NO: 21, 23, 25. In some embodiments, the cytoplasmic domain nucleotide sequence consists of SEQ ID NO:21, 23, 25.

In some embodiments, the cytoplasmic domain amino acid sequence has at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 amino acids, or any range or value derivable therein, and has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:22, 24, or 26. In some embodiments, the cytoplasmic domain amino acid sequence comprises SEQ ID NO: 22, 24, or 26. In some embodiments, the cytoplasmic domain amino acid sequence consists of SEQ ID NO:22, 24, or 26.

e. Hinge

In some embodiments of the CARs, there is a hinge region between the one or more extracellular antigen binding domains and the transmembrane domain. A hinge portion may link the antigen-binding domain to the transmembrane domain. It should be flexible enough to allow the antigen-binding domain to orient in different directions to facilitate antigen binding. As used herein, the term “hinge” refers to a flexible polypeptide connector region (used interchangeably herein with “hinge region” or “spacer”) providing structural flexibility and spacing to flanking polypeptide regions and can consist of natural or synthetic polypeptides. A “hinge” derived from an immunoglobulin (e.g., IgG1) is generally defined as stretching from Glu216 to Pro230 of human IgG1, for example (Burton (1985) Molec. Immunol., 22:161-206). Hinge regions of other IgG isotypes may be aligned with the IgG1 sequence by placing the first and last cysteine residues forming inter-heavy chain disulfide (S-S) bonds in the same positions. The hinge region may be of natural occurrence or non-natural occurrence, including but not limited to an altered hinge region as described in U.S. Pat. No. 5,677,425.

In specific embodiments, the hinge is of a particular length, such as 10-20, 10-15, 11-20, 11-15, 12-20, 12-15, or 15-20 amino acids in length, for example. The hinge portion of the construct can have a length of at least, at most, or exactly 4, 5, 6, 7, 8, 9, 10, 12, 15, 16, 17, 18, 19, 20, 20, 25, 30, 35, 40, 45, 50, 75, 100, 110, 119, 120, 130, 140, 150, 160, 170, 180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein). In some embodiments, the hinge portion consists of or comprises a hinge region from an immunoglobulin (e.g., IgG). Immunoglobulin hinge region amino acid sequences are known in the art; see, e.g., Tan et al. (1990) Proc. Natl. Acad. Sci. USA 87:162; and Huck et al. (1986) Nucl. Acids Res.

The hinge may be any suitable hinge and includes a hinge from IgG, or CD3, CD8, or CD28, in some cases. In specific embodiments, the hinge is a small flexible polypeptide that connects C_{H}2-C_{H}3 and C_{H}1 domains of IgG Fc. For example, one may utilize C_{H}2-C_{H}3 hinge (part or all) from various IgG subclasses (IgG1-4, either modified or not). However, in some cases the entire C_{H}2-C_{H}3 hinge is not utilized but instead a portion of the hinge is used (such as C_{H}3 by itself or part of C_{H}3 by itself). In particular embodiments, the C_{H}2-C_{H}3 hinge derived from IgG1 is utilized, and in some cases the entire C_{H}2-C_{H}3 hinge is used (all 229 amino acids), only the C_{H}3 hinge (119 amino acids) is used, or a short hinge (12 amino acids) is used. The hinge region can include a complete hinge region derived from an antibody of a different class or subclass from that of the C_{H}1 domain. The term “hinge” can also include regions derived from other receptors that provide a similar function in providing flexibility and spacing to flanking regions.

In specific cases, one can modify the identity or length of the spacer and/or hinge to optimize efficiency of the CAR. See, for example, Hudecek et al. (2014) and Jonnalagadda et al. (2015). The length of the hinge portion may have effects on the CAR's signaling activity and/or the CAR-T-cells' expansion properties in response to antigen-stimulated CAR signaling. In some embodiments, a shorter spacer such as less than 50, 45, 40, 30, 35, 30, 25, 20, 15, 14, 13, 12, 11, or 10 amino acids is used. In some embodiments, a longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids may have the advantage of increased expansion in vivo or in vitro.

Thus, in specific embodiments the IgG hinge region that is utilized is typically IgG1 or IgG4, and in some cases the CAR comprises the C_{H}2-C_{H}3 domain of IgG Fc. The use of the IgG Fc domain can provide flexibility to the CAR, has low immunogenicity, facilitates detection of CAR expression using anti-Fc reagents, and allows removal of one or more C_{H}2 or C_{H}3 modules to accommodate different spacer lengths. However, in one embodiment mutations in certain spacers to avoid FcγR binding may improve CAR+ T-cell engraftment and antitumor efficacy to avoid binding of soluble and cell surface Fc gamma receptors, for example, yet maintain the activity to mediate antigen-specific lysis. For example, one can employ IgG4-Fc spacers that have either been modified in the C_{H}2 region. For example, the C_{H}2 region may be mutated, including point mutations and/or deletions. Specific modifications have been demonstrated at two sites (L235E; N297Q) within the C_{H}2 region and/or incorporate a C_{H}2 deletion (Jonnalagadda et al, 2015). In specific embodiments, one may employ the IgG4 hinge-C_{H}2-C_{H}3 domain (229 aa in length) or only the hinge domain (12 aa in length) (Hudececk et al., 2015).

In specific embodiments, the hinge and/or spacer is from IgG, CD28, CD-8 alpha, 4-1BB, OX40, CD3ξ, T-cell receptor a or b chain, a CD3 ξchain, CD28, CD3e, CD45, CD4, CD5, CD8, CD9, CD16, CD22, CD33, CD37, CD64, CD80, CD86, CD134, CD137, ICOS, or CD154.

Examples of specific sequences of hinges that may be utilized include at least the following:

IgG hinge nucleotide sequence:
(SEQ ID NO: 27)
GTACGGTCACTGTCTCTTCACAGGATCCCGCCGAGCCCAAATCTCCTG
ACAAAACTCACACATGCCCACCGTGCCCAGCACCTGAACTCCTGGGGGG
ACCGTCAGTCTTCCTCTTCCCCCCAAAACCCAAGGACACCCTCATGATC
TCCCGGACCCCTGAGGTCACATGCGTGGTGGTGGACGTGAGCCACGAAG
ACCCTGAGGTCAAGTTCAACTGGTACGTGGACGGCGTGGAGGTGCATAA
TGCCAAGACAAAGCCGCGGGAGGAGCAGTACAACAGCACGTACCGTGTG
GTCAGCGTCCTCACCGTCCTGCACCAGGACTGGCTGAATGGCAAGGAGT
ACAAGTGCAAGGTCTCCAACAAAGCCCTCCCAGCCCCCATCGAGAAAAC
CATCTCCAAAGCCAAAGGGCAGCCCCGAGAACCACAGGTGTACACCCTG
CCCCCATCCCGGGATGAGCTGACCAAGAACCAGGTCAGCCTGACCTGCC
TGGTCAAAGGCTTCTATCCCAGCGACATCGCCGTGGAGTGGGAGAGCAA
TGGGCAACCGGAGAACAACTACAAGACCACGCCTCCCGTGCTGGACTCC
GACGGCTCCTTCTTCCTCTACAGCAAGCTCACCGTGGACAAGAGCAGGT
GGCAGCAGGGGAACGTCTTCTCATGCTCCGTGATGCATGAGGCTCTGCA
CAACCACTACACGCAGAAGAGCCTCTCCCTGTCTCCGGGTAAAAAAGAT
CCCAAATT.
IgG hinge amino acid sequence:
(SEQ ID NO: 28)
TVTVSSQDPAEPKSPDKTHTCPPCPAPELLGGPSVFLFPPKPKDTLMIS
RTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGKKDP
K.

Examples of particular hinge and/or spacer sequences that may be used include the following:

IgG4-derived hinge, IgG1 C_{H}3-derived spacer
nucleotide sequence:
(SEQ ID NO: 29)
GAGTCTAAATATGGCCCACCTTGCCCACCGTGCCCAGGGCAGCCCCGA
GAACCACAGGTGTACACCCTGCCCCCATCCCGGGATGAGCTGACCAAG
AACCAGGTCAGCCTGACCTGCCTGGTCAAAGGCTTCTATCCCAGCGAC
ATCGCCGTGGAGTGGGAGAGCAATGGGCAACCGGAGAACAACTACAAG
ACCACGCCTCCCGTGCTGGACTCCGACGGCTCCTTCTTCCTCTACAGC
AAGCTCACCGTGGACAAGAGCAGGTGGCAGCAGGGGAACGTCTTCTCA
TGCTCCGTGATGCATGAGGCTCTGCACAACgcCTACACGCAGAAGAGC
CTCTCCCTGTCTCCGGGTAAAAAAGATC.

An amino acid sequence translated from SEQ ID NO:29 is as follows:

(SEQ ID NO: 30)
ESKYGPPCPPCPGQPREPQVYTLPPSRDELTKNQVSLTCLVKGFYPSD
IAVEWESNGQPENNYKTTPPVLDSDGSFFLYSKLTVDKSRWQQGNVFS
CSVMHEALHNAYTQKSLSLSPGKKDPK.
CD8a-derived hinge nucleotide sequence:
(SEQ ID NO: 31)
CTGAGCAACTCCATCATGTACTTCAGCCACTTCGTGCCGGTCTTCCTG
CCAGCGAAGCCCACCACGACGCCAGCGCCGCGACCACCAACACCGGCG
CCCACCATCGCGTCGCAGCCCCTGTCCCTGCGCCCAGAGGCGTGCCGG
CCAGCGGCGGGGGGCGCAGTGCACACGAGGGGGCTGGACTTCG.

An amino acid sequence translated from SEQ ID NO:31 is as follows:

(SEQ ID NO: 32)
LSNSIMYFSHFVPVFLPAKPTTTPAPRPPTPAPTIASQPLSLRPEACR
PAAGGAVHTRGLDFA.

In some embodiments, the hinge and/or spacer nucleotide sequence has at least 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, or 750 nucleotides, or any range or value derivable therein, and has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:27, 29, or 31. In some embodiments, the hinge and/or spacer nucleotide sequence comprises SEQ ID NO:27, 29, or 31. In some embodiments, the hinge and/or spacer nucleotide sequence consists of SEQ ID NO:27, 29, or 31.

In some embodiments, the hinge and/or spacer amino acid sequence has at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, or 255 amino acids, or any range or value derivable therein, and has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO: 28, 30, or 32. In some embodiments, the hinge and/or spacer amino acid sequence comprises SEQ ID NO:28, 30, or 32. In some embodiments, the hinge and/or spacer amino acid sequence consists of SEQ ID NO:28, 30, or 32.

f. Therapeutic Controls

In some embodiments of the methods and compositions described herein, the CAR molecule is co-expressed with a therapeutic control.

Therapeutic controls regulate cell proliferation, facilitate cell selection (for example selecting cells which express the chimeric antigen receptors of the disclosure) or a combination thereof. In one embodiment, regulating cell proliferation comprises up-regulating cell proliferation to promote cell propagation. In another embodiment, regulating cell proliferation comprises down-regulating cell proliferation so as to reduce or inhibit cell propagation. In some embodiments, the agents that serve as therapeutic controls may promote enrichment of cells which express the chimeric antigen receptors which may result in a therapeutic advantage. In some embodiments, agents which serve as therapeutic controls may biochemically interact with additional compositions so as to regulate the functioning of the therapeutic controls. For example, EGFRt (a therapeutic control) may biochemically interact with cetuximab so as to regulate the function of EGFRt in selection, tracking, cell ablation or a combination thereof.

Exemplary therapeutic controls include truncated epidermal growth factor receptor (EGFRt), chimeric cytokine receptors (CCR) and/or dihydroxyfolate receptor (DHFR) (e.g., mutant DHFR). The polynucleotides encoding the CAR and the therapeutic control(s) may be linked via IRES sequences or via polynucleotide sequences encoding cleavable linkers. The CARs of the disclosure are constructed so that they may be expressed in cells, which in turn proliferate in response to the presence of at least one molecule that interacts with at least one antigen-specific targeting region, for instance, an antigen. In further embodiments, the therapeutic control comprises a cell-surface protein wherein the protein lacks intracellular signaling domains. It is contemplated that any cell surface protein lacking intracellular signaling or modified (e.g., by truncation) to lack intracellular signaling may be used. Further examples of a therapeutic control include truncated LNGFR, truncated CD19, and the like, wherein the truncated proteins lack intracellular signaling domains.

“Co-express” as used herein refers to simultaneous expression of two or more genes. Genes may be nucleic acids encoding, for example, a single protein or a chimeric protein as a single polypeptide chain. For example, the CARs of the disclosure may be co-expressed with a therapeutic control, wherein the CAR is encoded by a first polynucleotide chain and the therapeutic control is encoded by a second polynucleotide chain. In an embodiment, the first and second polynucleotide chains are linked by a nucleic acid sequence that encodes a cleavable linker. The polynucleotides encoding the CAR and the therapeutic control system may be linked by IRES sequences. Alternately, the CAR and the therapeutic control are encoded by two different polynucleotides that are not linked via a linker but are instead encoded by, for example, two different vectors. If the aforementioned sequences are encoded by separate vectors, these vectors may be simultaneously or sequentially transfected.

Further aspects of the therapeutic controls, CAR molecules, and methods of use for the compositions of the disclosure can be found in U.S. Pat. No. 9,447,194, which is herein incorporated by reference for all purposes.

g. Specific CAR Molecules

The disclosure also encompasses specific CAR molecules, including for expression in any type of immune cells.

In specific embodiments, a CD5 CAR molecule is utilized, as follows:

(SEQ ID NO: 33)
ATGGAGTTTGGGCTGAGCTGGCTTTTTCTTGTGGCTATTTTAAAAGGT
GTCCAGTGCATCGATGCCATGGGCAACATCCAGCTGGTGCAGAGCGGC
CCTGAGCTGAAGAAACCCGGCGAGACAGTGAAGATCAGCTGCAAGGCC
AGCGGCTACACCTTCACCAACTACGGCATGAACTGGGTGAAACAGGCC
CCAGGCAAGGGCCTGCGGTGGATGGGCTGGATCAACACCCACACCGGC
GAGCCCACCTACGCCGACGACTTCAAGGGCAGATTCGCCTTCAGCCTG
GAAACCAGCGCCAGCACCGCCTACCTGCAGATCAACAACCTGAAGAAC
GAGGACACCGCCACCTATTTCTGCACCAGACGGGGCTACGACTGGTAC
TTCGACGTGTGGGGAGCCGGCACCACCGTGACCGTGTCTAGCGGAGGC
GGAGGATCTGGCGGAGGGGGATCAGGCGGCGGAGGCAGCGACATCAAG
ATGACCCAGAGCCCCAGCTCTATGTACGCCAGCCTGGGCGAGCGCGTG
ACCATCACATGCAAGGCCTCCCAGGACATCAACAGCTACCTGAGCTGG
TTCCACCACAAGCCCGGCAAGAGCCCCAAGACCCTGATCTACCGGGCC
AACCGGCTGGTGGACGGCGTGCCAAGCAGATTCAGCGGCAGCGGCTCC
GGCCAGGACTACAGCCTGACCATCAGCAGCCTGGACTACGAGGACATG
GGCATCTACTACTGCCAGCAGTACGACGAGAGCCCCTGGACCTTCGGA
GGCGGCACCAAGCTGGAAATGAAGGGCAGCGGGGATCCCGCCGAGTCT
AAATATGGCCCACCTTGCCCACCGTGCCCAGGGCAGCCCCGAGAACCA
CAGGTGTACACCCTGCCCCCATCCCGGGATGAGCTGACCAAGAACCAG
GTCAGCCTGACCTGCCTGGTCAAAGGCTTCTATCCCAGCGACATCGCC
GTGGAGTGGGAGAGCAATGGGCAACCGGAGAACAACTACAAGACCACG
CCTCCCGTGCTGGACTCCGACGGCTCCTTCTTCCTCTACAGCAAGCTC
ACCGTGGACAAGAGCAGGTGGCAGCAGGGGAACGTCTTCTCATGCTCC
GTGATGCATGAGGCTCTGCACAACGCCTACACGCAGAAGAGCCTCTCC
CTGTCTCCGGGTAAAAAAGATCCCAAATTTTGGGTGCTGGTGGTGGTT
GGTGGAGTCCTGGCTTGCTATAGCTTGCTAGTAACAGTGGCCTTTATT
ATTTTCTGGGTGAGGAGTAAGAGGAGCAGGCTCCTGCACAGTGACTAC
ATGAACATGACTCCCCGCCGCCCCGGGCCCACCCGCAAGCATTACCAG
CCCTATGCCCCACCACGCGACTTCGCAGCCTATCGCTCCAGAGTGAAG
TTCAGCAGGAGCGCAGACGCCCCCGCGTACCAGCAGGGCCAGAACCAG
CTCTATAACGAGCTCAATCTAGGACGAAGAGAGGAGTACGATGTTTTG
GACAAGAGACGTGGCCGGGACCCTGAGATGGGGGGAAAGCCGAGAAGG
AAGAACCCTCAGGAAGGCCTGTACAATGAACTGCAGAAAGATAAGATG
GCGGAGGCCTACAGTGAGATTGGGATGAAAGGCGAGCGCCGGAGGGGC
AAGGGGCACGATGGCCTTTACCAGGGTCTCAGTACAGCCACCAAGGAC
ACCTACGACGCCCTTCACATGCAGGCCCTGCCTCCTCGC

An amino acid sequence translated from SEQ ID NO:33 is as follows:

(SEQ ID NO: 34)
MEFGLSWLFLVAILKGVQCIDAMGNIQLVQSGPELKKPGETVKISCKA
SGYTFTNYGMNWVKQAPGKGLRWMGWINTHTGEPTYADDFKGRFAFSL
ETSASTAYLQINNLKNEDTATYFCTRRGYDWYFDVWGAGTTVTVSSGG
GGSGGGGSGGGGSDIKMTQSPSSMYASLGERVTITCKASQDINSYLSW
FHHKPGKSPKTLIYRANRLVDGVPSRFSGSGSGQDYSLTISSLDYEDM
GIYYCQQYDESPWTFGGGTKLEMKGSGDPAESKYGPPCPPCPGQPREP
QVYTLPPSRDELTKNQVSLTCLVKGFYPSDIAVEWESNGQPENNYKTT
PPVLDSDGSFFLYSKLTVDKSRWQQGNVFSCSVMHEALHNAYTQKSLS
LSPGKKDPKFWVLVVVGGVLACYSLLVTVAFIIFWVRSKRSRLLHSDY
MNMTPRRPGPTRKHYQPYAPPRDFAAYRSRVKFSRSADAPAYQQGQNQ
LYNELNLGRREEYDVLDKRRGRDPEMGGKPRRKNPQEGLYNELQKDKM
AEAYSEIGMKGERRRGKGHDGLYQGLSTATKDTYDALHMQALPPR

In specific embodiments, a CD7 CAR molecule is utilized, as follows:

(SEQ ID NO: 35)
ATGGCCTTACCAGTGACCGCCTTGCTCCTGCCGCTGGCCTTGCTGCTC
CACGCCGCCAGGCCGCAGGTGAAGCTGCAGGAGTCAGGGGGAGGCTTA
GTGAAGCCTGGAGGGTCCCTGAAACTCTCCTGTGCAGCCTCTGGATTC
ACTTTCAGTAGCTATGCAATGTCTTGGGTTCGCCAGACTCCGGAGAAG
AGGCTGGAGTGGGTCGCAACCATTAGTAGTGGTGGTAGTTACACCTAC
TATCCAGACAGTGTGAAGGGGCGATTCACCATCTCCAGAGACAATGCC
AAGAACACCCTGTACCTGCAAATGAGCAGTCTGAGGTCTGAGGACACG
GCCATGTATTACTGTGCAAGACAGGATGGTTACTACCCGGGCTGGTTT
GCTAACTGGGGGCAAGGGACCACGGTCACCGTCTCCTCAGGTGGAGGC
GGTTCAGGCGGAGGTGGCTCTGGCGGTGGCGGATCGGACATCGAGCTC
ACTCAGTCTCCAGCAATCATGTCTGCATCTCTAGGGGAGGAGATCACC
CTAACCTGCAGTGCCAGCTCCAGTGTAAGTTACATGCACTGGTACCAG
CAGAAGTCAGGCACTTCTCCCAAACTCTTGATTTATAGCACATCCAAC
CTGGCTTCTGGAGTCCCTTCTCGCTTCAGTGGCAGTGGGTCTGGGACC
TTTTATTCTCTCACAATCAGCAGTGTGGAGGCTGAAGATGCTGCCGAT
TATTACTGCCATCAGTGGAGTAGTTACACGTTCGGAGGGGGCACCAAG
CTGGAAATCAAACGGGCGGATCCCGCCGAGTCTAAATATGGCCCACCT
TGCCCACCGTGCCCAGGGCAGCCCCGAGAACCACAGGTGTACACCCTG
CCCCCATCCCGGGATGAGCTGACCAAGAACCAGGTCAGCCTGACCTGC
CTGGTCAAAGGCTTCTATCCCAGCGACATCGCCGTGGAGTGGGAGAGC
AATGGGCAACCGGAGAACAACTACAAGACCACGCCTCCCGTGCTGGAC
TCCGACGGCTCCTTCTTCCTCTACAGCAAGCTCACCGTGGACAAGAGC
AGGTGGCAGCAGGGGAACGTCTTCTCATGCTCCGTGATGCATGAGGCT
CTGCACAACGCCTACACGCAGAAGAGCCTCTCCCTGTCTCCGGGTAAA
AAAGATCCCAAATTTTGGGTGCTGGTGGTGGTTGGTGGAGTCCTGGCT
TGCTATAGCTTGCTAGTAACAGTGGCCTTTATTATTTTCTGGGTGAGG
AGTAAGAGGAGCAGGCTCCTGCACAGTGACTACATGAACATGACTCCC
CGCCGCCCCGGGCCCACCCGCAAGCATTACCAGCCCTATGCCCCACCA
CGCGACTTCGCAGCCTATCGCTCCAGAGTGAAGTTCAGCAGGAGCGCA
GACGCCCCCGCGTACCAGCAGGGCCAGAACCAGCTCTATAACGAGCTC
AATCTAGGACGAAGAGAGGAGTACGATGTTTTGGACAAGAGACGTGGC
CGGGACCCTGAGATGGGGGGAAAGCCGAGAAGGAAGAACCCTCAGGAA
GGCCTGTACAATGAACTGCAGAAAGATAAGATGGCGGAGGCCTACAGT
GAGATTGGGATGAAAGGCGAGCGCCGGAGGGGCAAGGGGCACGATGGC
CTTTACCAGGGTCTCAGTACAGCCACCAAGGACACCTACGACGCCCTT
CACATGCAGGCCCTGCCCCCTCGC

An amino acid sequence translated from SEQ ID NO:35 is as follows:

(SEQ ID NO: 36)
MALPVTALLLPLALLLHAARPQVKLQESGGGLVKPGGSLKLSCAASGF
TFSSYAMSWVRQTPEKRLEWVATISSGGSYTYYPDSVKGRFTISRDNA
KNTLYLQMSSLRSEDTAMYYCARQDGYYPGWFANWGQGTTVTVSSGGG
GSGGGGSGGGGSDIELTQSPAIMSASLGEEITLTCSASSSVSYMHWYQ
QKSGTSPKLLIYSTSNLASGVPSRFSGSGSGTFYSLTISSVEAEDAAD
YYCHQWSSYTFGGGTKLEIKRADPAESKYGPPCPPCPGQPREPQVYTL
PPSRDELTKNQVSLTCLVKGFYPSDIAVEWESNGQPENNYKTTPPVLD
SDGSFFLYSKLTVDKSRWQQGNVFSCSVMHEALHNAYTQKSLSLSPGK
KDPKFWVLVVVGGVLACYSLLVTVAFIIFWVRSKRSRLLHSDYMNMTP
RRPGPTRKHYQPYAPPRDFAAYRSRVKESRSADAPAYQQGQNQLYNEL
NLGRREEYDVLDKRRGRDPEMGGKPRRKNPQEGLYNELQKDKMAEAYS
EIGMKGERRRGKGHDGLYQGLSTATKDTYDALHMQALPPR

In specific embodiments, a CD7 CAR molecule is utilized, as follows:

(SEQ ID NO: 37)
ATGGCCCTGCCTGTGACCGCTCTGCTGCTGCCTCTGGCACTGCTGCTG
CACGCTGCTAGACCTGGCGCTCAGCCTGCTATGGCCGCCTACAAGGAC
ATCCAGATGACCCAGACCACCAGCAGCCTGTCTGCCAGCCTGGGCGAC
AGAGTGACCATCAGCTGTAGCGCCAGCCAGGGCATCAGCAACTACCTG
AACTGGTATCAGCAGAAACCCGACGGCACCGTGAAGCTGCTGATCTAC
TACACCAGCTCCCTGCACAGCGGCGTGCCCAGCAGATTTTCTGGCAGC
GGCTCCGGCACCGACTACAGCCTGACCATCTCCAACCTGGAACCCGAG
GATATCGCCACCTACTACTGCCAGCAGTACAGCAAGCTGCCCTACACC
TTCGGCGGAGGCACCAAGCTGGAAATCAAGAGGGGAGGCGGAGGAAGC
GGAGGCGGTGGATCTGGTGGTGGCGGTTCTGGCGGAGGTGGAAGCGAA
GTGCAGCTGGTGGAATCTGGCGGCGGACTGGTCAAGCCTGGCGGCTCT
CTGAAACTGAGCTGTGCCGCCTCTGGCCTGACCTTCAGCAGCTACGCT
ATGAGCTGGGTGCGCCAGACCCCCGAGAAGAGACTGGAATGGGTGGCC
AGCATCAGCAGCGGCGGCTTTACCTACTACCCCGACAGCGTGAAGGGC
CGGTTCACCATCAGCCGGGACAACGCCCGGAACATCCTGTACCTGCAG
ATGAGCAGCCTGCGGAGCGAGGACACCGCCATGTACTACTGCGCCAGG
GATGAAGTGCGGGGCTACCTGGATGTGTGGGGAGCCGGAACAACCGTG
ACCGTGTCTAGTGCCAGCGGAGCGGATCCCGCCGAGTCTAAATATGGC
CCACCTTGCCCACCGTGCCCAGGGCAGCCCCGAGAACCACAGGTGTAC
ACCCTGCCCCCATCCCGGGATGAGCTGACCAAGAACCAGGTCAGCCTG
ACCTGCCTGGTCAAAGGCTTCTATCCCAGCGACATCGCCGTGGAGTGG
GAGAGCAATGGGCAACCGGAGAACAACTACAAGACCACGCCTCCCGTG
CTGGACTCCGACGGCTCCTTCTTCCTCTACAGCAAGCTCACCGTGGAC
AAGAGCAGGTGGCAGCAGGGGAACGTCTTCTCATGCTCCGTGATGCAT
GAGGCTCTGCACAACGCCTACACGCAGAAGAGCCTCTCCCTGTCTCCG
GGTAAAAAAGATCCCAAATTTTGGGTGCTGGTGGTGGTTGGTGGAGTC
CTGGCTTGCTATAGCTTGCTAGTAACAGTGGCCTTTATTATTTTCTGG
GTGAGGAGTAAGAGGAGCAGGCTCCTGCACAGTGACTACATGAACATG
ACTCCCCGCCGCCCCGGGCCCACCCGCAAGCATTACCAGCCCTATGCC
CCACCACGCGACTTCGCAGCCTATCGCTCCAGAGTGAAGTTCAGCAGG
AGCGCAGACGCCCCCGCGTACCAGCAGGGCCAGAACCAGCTCTATAAC
GAGCTCAATCTAGGACGAAGAGAGGAGTACGATGTTTTGGACAAGAGA
CGTGGCCGGGACCCTGAGATGGGGGGAAAGCCGAGAAGGAAGAACCCT
CAGGAAGGCCTGTACAATGAACTGCAGAAAGATAAGATGGCGGAGGCC
TACAGTGAGATTGGGATGAAAGGCGAGCGCCGGAGGGGCAAGGGGCAC
GATGGCCTTTACCAGGGTCTCAGTACAGCCACCAAGGACACCTACGAC
GCCCTTCACATGCAGGCCCTGCCCCCTCGC

An amino acid sequence translated from SEQ ID NO:37 is as follows:

(SEQ ID NO: 38)
MALPVTALLLPLALLLHAARPGAQPAMAAYKDIQMTQTTSSLSASLGD
RVTISCSASQGISNYLNWYQQKPDGTVKLLIYYTSSLHSGVPSRFSGS
GSGTDYSLTISNLEPEDIATYYCQQYSKLPYTFGGGTKLEIKRGGGGS
GGGGSGGGGSGGGGSEVQLVESGGGLVKPGGSLKLSCAASGLTFSSYA
MSWVRQTPEKRLEWVASISSGGFTYYPDSVKGRFTISRDNARNILYLQ
MSSLRSEDTAMYYCARDEVRGYLDVWGAGTTVTVSSASGADPAESKYG
PPCPPCPGQPREPQVYTLPPSRDELTKNQVSLTCLVKGFYPSDIAVEW
ESNGQPENNYKTTPPVLDSDGSFFLYSKLTVDKSRWQQGNVFSCSVMH
EALHNAYTQKSLSLSPGKKDPKFWVLVVVGGVLACYSLLVTVAFIIFW
VRSKRSRLLHSDYMNMTPRRPGPTRKHYQPYAPPRDFAAYRSRVKFSR
SADAPAYQQGQNQLYNELNLGRREEYDVLDKRRGRDPEMGGKPRRKNP
QEGLYNELQKDKMAEAYSEIGMKGERRRGKGHDGLYQGLSTATKDTYD
ALHMQALPPR

In specific embodiments, a CD7 CAR molecule is utilized, as follows:

(SEQ ID NO: 39)
ATGGCCTTACCAGTGACCGCCTTGCTCCTGCCGCTGGCCTTGCTGCTC
CACGCCGCCAGGCCGCAGGTCCAGCTGCAGGAGTCTGGGGCTGAACTG
GTGAAGCCTGGGGCTTCAGTGAAGCTGTCCTGCAAGGCTTCTGGCTAC
ACCTTCACGAGCTACTGGATGCACTGGGTGAAGCAGAGGCCTGGACAA
GGCCTTGAGTGGATTGGAAAGATTAATCCTAGCAACGGTCGTACTAAC
TACAATGAGAAGTTCAAGAGCAAGGCCACACTGACTGTAGACAAATCC
TCCAGCACAGCCTACATGCAACTCAGCAGCCTGACATCTGAGGACTCT
GCGGTCTATTACTGTGCAAGAGGGGGAGTCTACTATGACCTTTATTAC
TATGCTCTGGACTACTGGGGCCAAGGCACCACGGTCACCGTCTCCTCA
GGTGGAGGCGGTTCAGGCGGAGGTGGCTCTGGCGGTGGCGGATCGGAC
ATCGAGCTCACTCAGTCTCCAGCCACCCTGTCTGTGACTCCAGGAGAT
AGCGTCAGTCTTTCCTGCAGGGCCAGCCAAAGTATTAGCAACAACCTA
CACTGGTATCAACAAAAATCACATGAGTCTCCAAGGCTTCTCATCAAG
TCTGCTTCCCAGTCCATCTCTGGAATCCCCTCCAGGTTCAGTGGCAGT
GGATCAGGGACAGATTTCACTCTCAGTATCAACAGTGTGGAGACTGAA
GATTTTGGAATGTATTTCTGTCAACAGAGTAACAGCTGGCCGTACACG
TTCGGAGGGGGGACAAAGTTGGAAATAAAACGGGCGGATCCCGCCGAG
TCTAAATATGGCCCACCTTGCCCACCGTGCCCAGGGCAGCCCCGAGAA
CCACAGGTGTACACCCTGCCCCCATCCCGGGATGAGCTGACCAAGAAC
CAGGTCAGCCTGACCTGCCTGGTCAAAGGCTTCTATCCCAGCGACATC
GCCGTGGAGTGGGAGAGCAATGGGCAACCGGAGAACAACTACAAGACC
ACGCCTCCCGTGCTGGACTCCGACGGCTCCTTCTTCCTCTACAGCAAG
CTCACCGTGGACAAGAGCAGGTGGCAGCAGGGGAACGTCTTCTCATGC
TCCGTGATGCATGAGGCTCTGCACAACGCCTACACGCAGAAGAGCCTC
TCCCTGTCTCCGGGTAAAAAAGATCCCAAATTTTGGGTGCTGGTGGTG
GTTGGTGGAGTCCTGGCTTGCTATAGCTTGCTAGTAACAGTGGCCTTT
ATTATTTTCTGGGTGAGGAGTAAGAGGAGCAGGCTCCTGCACAGTGAC
TACATGAACATGACTCCCCGCCGCCCCGGGCCCACCCGCAAGCATTAC
CAGCCCTATGCCCCACCACGCGACTTCGCAGCCTATCGCTCCAGAGTG
AAGTTCAGCAGGAGCGCAGACGCCCCCGCGTACCAGCAGGGCCAGAAC
CAGCTCTATAACGAGCTCAATCTAGGACGAAGAGAGGAGTACGATGTT
TTGGACAAGAGACGTGGCCGGGACCCTGAGATGGGGGGAAAGCCGAGA
AGGAAGAACCCTCAGGAAGGCCTGTACAATGAACTGCAGAAAGATAAG
ATGGCGGAGGCCTACAGTGAGATTGGGATGAAAGGCGAGCGCCGGAGG
GGCAAGGGGCACGATGGCCTTTACCAGGGTCTCAGTACAGCCACCAAG
GACACCTACGACGCCCTTCACATGCAGGCCCTGCCCCCTCGC

An amino acid sequence translated from SEQ ID NO:39 is as follows:

(SEQ ID NO: 40)
MALPVTALLLPLALLLHAARPQVQLQESGAELVKPGASVKLSCKASGY
TFTSYWMHWVKQRPGQGLEWIGKINPSNGRTNYNEKFKSKATLTVDKS
SSTAYMQLSSLTSEDSAVYYCARGGVYYDLYYYALDYWGQGTTVTVSS
GGGGSGGGGSGGGGSDIELTQSPATLSVTPGDSVSLSCRASQSISNNL
HWYQQKSHESPRLLIKSASQSISGIPSRFSGSGSGTDFTLSINSVETE
DFGMYFCQQSNSWPYTFGGGTKLEIKRADPAESKYGPPCPPCPGQPRE
PQVYTLPPSRDELTKNQVSLTCLVKGFYPSDIAVEWESNGQPENNYKT
TPPVLDSDGSFFLYSKLTVDKSRWQQGNVFSCSVMHEALHNAYTQKSL
SLSPGKKDPKFWVLVVVGGVLACYSLLVTVAFIIFWVRSKRSRLLHSD
YMNMTPRRPGPTRKHYQPYAPPRDFAAYRSRVKFSRSADAPAYQQGQN
QLYNELNLGRREEYDVLDKRRGRDPEMGGKPRRKNPQEGLYNELQKDK
MAEAYSEIGMKGERRRGKGHDGLYQGLSTATKDTYDALHMQALPPR

In specific embodiments, a CD2 CAR molecule is utilized, as follows:

(SEQ ID NO: 41)
ATGGCCTTACCAGTGACCGCCTTGCTCCTGCCGCTGGCCTTGCTGCTC
CACGCCGCCAGGCCGGATGTTGTTCTTACTCAGACTCCACCAACTTTG
TTGGCAACAATTGGGCAAAGTGTGTCAATTAGTTGCAGATCAAGCCAA
AGTCTCTTGCACAGTAGCGGAAATACCTATCTGAACTGGCTGTTGCAG
CGGACTGGGCAATCCCCGCAACCGCTCATATACCTGGTAAGCAAGCTA
GAGTCAGGGGTGCCGAATCGCTTCTCCGGATCCGGTAGTGGTACGGAT
TTCACGCTGAAGATAAGCGGAGTGGAAGCGGAAGACTTGGGCGTGTAC
TACTGTATGCAGTTCACACACTATCCCTACACTTTTGGGGGGGGTACT
AAACTTGAGCTTAAGTCTGGAGGCGGTGGATCTGGCGGTGGAGGTAGC
GGAGGAGGCGGTAGCGAAGTGCAATTGCAGCAGTCAGGGCCAGAGCTG
CAAAGACCTGGTGCCAGCGTGAAGTTGTCCTGTAAAGCCTCCGGTTAT
ATCTTCACAGAGTACTATATGTACTGGGTTAAGCAACGCCCAAAACAA
GGCCTGGAGCTTGTGGGCCGAATCGACCCCGAAGATGGTTCTATTGAC
TACGTAGAGAAGTTCAAGAAAAAGGCAACACTCACTGCGGACACTAGT
TCAAACACTGCCTACATGCAGCTCTCTAGCCTGACATCCGAAGACACC
GCCACGTATTTTTGCGCACGAGGTAAATTCAACTATCGCTTCGCATAC
TGGGGGCAGGGTACTCTCGTCACCGTCTCCTCAGAGTCTAAATATGGC
CCACCTTGCCCACCGTGCCCAGGGCAGCCCCGAGAACCACAGGTGTAC
ACCCTGCCCCCATCCCGGGATGAGCTGACCAAGAACCAGGTCAGCCTG
ACCTGCCTGGTCAAAGGCTTCTATCCCAGCGACATCGCCGTGGAGTGG
GAGAGCAATGGGCAACCGGAGAACAACTACAAGACCACGCCTCCCGTG
CTGGACTCCGACGGCTCCTTCTTCCTCTACAGCAAGCTCACCGTGGAC
AAGAGCAGGTGGCAGCAGGGGAACGTCTTCTCATGCTCCGTGATGCAT
GAGGCTCTGCACAACGCCTACACGCAGAAGAGCCTCTCCCTGTCTCCG
GGTAAAAAAGATCCCAAATTTTGGGTGCTGGTGGTGGTTGGTGGAGTC
CTGGCTTGCTATAGCTTGCTAGTAACAGTGGCCTTTATTATTTTCTGG
GTGAGGAGTAAGAGGAGCAGGCTCCTGCACAGTGACTACATGAACATG
ACTCCCCGCCGCCCCGGGCCCACCCGCAAGCATTACCAGCCCTATGCC
CCACCACGCGACTTCGCAGCCTATCGCTCCAGAGTGAAGTTCAGCAGG
AGCGCAGACGCCCCCGCGTACCAGCAGGGCCAGAACCAGCTCTATAAC
GAGCTCAATCTAGGACGAAGAGAGGAGTACGATGTTTTGGACAAGAGA
CGTGGCCGGGACCCTGAGATGGGGGGAAAGCCGAGAAGGAAGAACCCT
CAGGAAGGCCTGTACAATGAACTGCAGAAAGATAAGATGGCGGAGGCC
TACAGTGAGATTGGGATGAAAGGCGAGCGCCGGAGGGGCAAGGGGCAC
GATGGCCTTTACCAGGGTCTCAGTACAGCCACCAAGGACACCTACGAC
GCCCTTCACATGCAGGCCCTGCCTCCTCGCTAA

An amino acid sequence translated from SEQ ID NO:41 is as follows:

(SEQ ID NO: 42)
MALPVTALLLPLALLLHAARPDVVLTQTPPTLLATIGQSVSISCRSSQ
SLLHSSGNTYLNWLLQRTGQSPQPLIYLVSKLESGVPNRFSGSGSGTD
FTLKISGVEAEDLGVYYCMQFTHYPYTFGAGTKLELKSGGGGSGGGGS
GGGGSEVQLQQSGPELQRPGASVKLSCKASGYIFTEYYMYWVKQRPKQ
GLELVGRIDPEDGSIDYVEKFKKKATLTADTSSNTAYMQLSSLTSEDT
ATYFCARGKFNYRFAYWGQGTLVTVSSESKYGPPCPPCPGQPREPQVY
TLPPSRDELTKNQVSLTCLVKGFYPSDIAVEWESNGQPENNYKTTPPV
LDSDGSFFLYSKLTVDKSRWQQGNVFSCSVMHEALHNAYTQKSLSLSP
GKKDPKFWVLVVVGGVLACYSLLVTVAFIIFWVRSKRSRLLHSDYMNM
TPRRPGPTRKHYQPYAPPRDFAAYRSRVKFSRSADAPAYQQGQNQLYN
ELNLGRREEYDVLDKRRGRDPEMGGKPRRKNPQEGLYNELQKDKMAEA
YSEIGMKGERRRGKGHDGLYQGLSTATKDTYDALHMQALPPR

In specific embodiments, a CD2 CAR molecule is utilized, as follows:

(SEQ ID NO: 43)
ATGGCCTTACCAGTGACCGCCTTGCTCCTGCCGCTGGCCTTGCTGCTC
CACGCCGCCAGGCCGGAAGTGCAATTGCAGCAGTCAGGGCCAGAGCTG
CAAAGACCTGGTGCCAGCGTGAAGTTGTCCTGTAAAGCCTCCGGTTAT
ATCTTCACAGAGTACTATATGTACTGGGTTAAGCAACGCCCAAAACAA
GGCCTGGAGCTTGTGGGCCGAATCGACCCCGAAGATGGTTCTATTGAC
TACGTAGAGAAGTTCAAGAAAAAGGCAACACTCACTGCGGACACTAGT
TCAAACACTGCCTACATGCAGCTCTCTAGCCTGACATCCGAAGACACC
GCCACGTATTTTTGCGCACGAGGTAAATTCAACTATCGCTTCGCATAC
TGGGGGCAGGGTACTCTCGTCACCGTCTCCTCATCTGGAGGCGGTGGA
TCTGGCGGTGGAGGTAGCGGAGGAGGCGGTAGCGATGTTGTTCTTACT
CAGACTCCACCAACTTTGTTGGCAACAATTGGGCAAAGTGTGTCAATT
AGTTGCAGATCAAGCCAAAGTCTCTTGCACAGTAGCGGAAATACCTAT
CTGAACTGGCTGTTGCAGCGGACTGGGCAATCCCCGCAACCGCTCATA
TACCTGGTAAGCAAGCTAGAGTCAGGGGTGCCGAATCGCTTCTCCGGA
TCCGGTAGTGGTACGGATTTCACGCTGAAGATAAGCGGAGTGGAAGCG
GAAGACTTGGGCGTGTACTACTGTATGCAGTTCACACACTATCCCTAC
ACTTTTGGGGGGGGTACTAAACTTGAGCTTAAGGAGTCTAAATATGGC
CCACCTTGCCCACCGTGCCCAGGGCAGCCCCGAGAACCACAGGTGTAC
ACCCTGCCCCCATCCCGGGATGAGCTGACCAAGAACCAGGTCAGCCTG
ACCTGCCTGGTCAAAGGCTTCTATCCCAGCGACATCGCCGTGGAGTGG
GAGAGCAATGGGCAACCGGAGAACAACTACAAGACCACGCCTCCCGTG
CTGGACTCCGACGGCTCCTTCTTCCTCTACAGCAAGCTCACCGTGGAC
AAGAGCAGGTGGCAGCAGGGGAACGTCTTCTCATGCTCCGTGATGCAT
GAGGCTCTGCACAACGCCTACACGCAGAAGAGCCTCTCCCTGTCTCCG
GGTAAAAAAGATCCCAAATTTTGGGTGCTGGTGGTGGTTGGTGGAGTC
CTGGCTTGCTATAGCTTGCTAGTAACAGTGGCCTTTATTATTTTCTGG
GTGAGGAGTAAGAGGAGCAGGCTCCTGCACAGTGACTACATGAACATG
ACTCCCCGCCGCCCCGGGCCCACCCGCAAGCATTACCAGCCCTATGCC
CCACCACGCGACTTCGCAGCCTATCGCTCCAGAGTGAAGTTCAGCAGG
AGCGCAGACGCCCCCGCGTACCAGCAGGGCCAGAACCAGCTCTATAAC
GAGCTCAATCTAGGACGAAGAGAGGAGTACGATGTTTTGGACAAGAGA
CGTGGCCGGGACCCTGAGATGGGGGGAAAGCCGAGAAGGAAGAACCCT
CAGGAAGGCCTGTACAATGAACTGCAGAAAGATAAGATGGCGGAGGCC
TACAGTGAGATTGGGATGAAAGGCGAGCGCCGGAGGGGCAAGGGGCAC
GATGGCCTTTACCAGGGTCTCAGTACAGCCACCAAGGACACCTACGAC
GCCCTTCACATGCAGGCCCTGCCTCCTCGC

An amino acid sequence translated from SEQ ID NO:43 is as follows:

(SEQ ID NO: 44)
MALPVTALLLPLALLLHAARPEVQLQQSGPELQRPGASVKLSCKASGY
IFTEYYMYWVKQRPKQGLELVGRIDPEDGSIDYVEKFKKKATLTADTS
SNTAYMQLSSLTSEDTATYFCARGKFNYRFAYWGQGTLVTVSSSGGGG
SGGGGSGGGGSDVVLTQTPPTLLATIGQSVSISCRSSQSLLHSSGNTY
LNWLLQRTGQSPQPLIYLVSKLESGVPNRFSGSGSGTDFTLKISGVEA
EDLGVYYCMQFTHYPYTFGAGTKLELKESKYGPPCPPCPGQPREPQVY
TLPPSRDELTKNQVSLTCLVKGFYPSDIAVEWESNGQPENNYKTTPPV
LDSDGSFFLYSKLTVDKSRWQQGNVFSCSVMHEALHNAYTQKSLSLSP
GKKDPKFWVLVVVGGVLACYSLLVTVAFIIFWVRSKRSRLLHSDYMNM
TPRRPGPTRKHYQPYAPPRDFAAYRSRVKFSRSADAPAYQQGQNQLYN
ELNLGRREEYDVLDKRRGRDPEMGGKPRRKNPQEGLYNELQKDKMAEA
YSEIGMKGERRRGKGHDGLYQGLSTATKDTYDALHMQALPPR

In some embodiments, the CAR molecule nucleotide sequence has at least 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1050, 1100, 1150, 1200, 1250, 1300, 1350, 1400, 1450, 1500, 1550, 1600, 1650, 1700, 1750, or 1800 nucleotides, or any range or value derivable therein, and has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:33, 35, 37, 39, 41, or 43. In some embodiments, the CAR molecule nucleotide sequence comprises SEQ ID NO:33, 35, 37, 39, 41, or 43. In some embodiments, the CAR molecule nucleotide sequence consists of SEQ ID NO:33, 35, 37, 39, 41, or 43.

In some embodiments, the CAR molecule amino acid sequence has at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 350, 400, 500, 550, or 600 amino acids, or any range or value derivable therein, and has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO: 34, 36, 38, 40, 42, or 44. In some embodiments, the CAR molecule amino acid sequence comprises SEQ ID NO:34, 36, 38, 40, 42, or 44. In some embodiments, the CAR molecule amino acid sequence consists of SEQ ID NO:34, 36, 38, 40, 42, or 44.

### B. T Cell Receptors (TCRs)

In some embodiments, a cancer cell, infectious disease, and/or immune disorder antigen-targeting genetically engineered antigen receptor includes recombinant TCRs and/or TCRs cloned from naturally occurring T-cells. A “T-cell receptor” or “TCR” refers to a molecule that contains a variable a and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form.

Typically, TCRs that exist in αβ and γδ forms are generally structurally similar, but T-cells expressing them may have distinct anatomical locations or functions. A TCR can be found on the surface of a cell or in soluble form. Generally, a TCR is found on the surface of T-cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e., MHC-peptide complex. An “antigen-binding portion” or antigen-binding fragment” of a TCR, which can be used interchangeably, refers to a molecule that contains a portion of the structural domains of a TCR, but that binds the antigen (e.g., MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., Vₐ or Vₚ; typically amino acids 1 to 116 based on Kabat numbering Kabat et al., “Sequences of Proteins of Immunological Interest, US Dept. Health and Human Services, Public Health Service National Institutes of Health, 1991, 5th ed.) at the N-terminus, and one constant domain (e.g., a-chain constant domain or Cₐ, typically amino acids 117 to 259 based on Kabat, β-chain constant domain or Cₚ, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the a and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ξ-chain. For example, in mammals the complex can contain a CD3γ chain, a CD3δchain, two CD3ε chains, and a homodimer of CD3ξ chains. The CD3γ, CD3δ, and CD3& chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T-cell receptor chains. The intracellular tails of the CD3γ, CD3ε, and CD3E chains each contain a single conserved motif known as an immunoreceptor tyrosine-based activation motif or ITAM, whereas each CD3ξ chain has three. Generally, ITAMs are involved in the signaling capacity of the TCR complex. These accessory molecules have negatively charged transmembrane regions and play a role in propagating the signal from the TCR into the cell. The CD3- and ξ-chains, together with the TCR, form what is known as the T-cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T-cell (e.g., cytotoxic T-cell), T-cell hybridomas or other publicly available source. In some embodiments, the T-cells can be obtained from in vivo isolated cells. In some embodiments, a high-affinity T-cell clone can be isolated from a patient, and the TCR isolated. In some embodiments, the T-cells can be a cultured T-cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

### III. Immune Cells

Disclosed herein are methods and compositions which utilize genetically engineered immune cells. The present disclosure encompasses immune cells of any kind that harbor at least one vector that encodes at least one antigen-targeting receptor that recognizes at least one target antigen, for example, a cancer cell antigen, an infectious disease antigen, and/or an immune disorder antigen. Thus, “genetically engineered immune cells” or “engineered immune cells” are immune cells that have been manipulated to express one or more antigen-targeting receptors that recognize one or more target antigen. Any type of immune cells may be utilized in the methods and compositions of the disclosure. In some embodiments, the genetically engineered immune cells are αβ−T-cells, γδ−T-cells, regulatory T-cells, Natural Killer (NK) cells, Natural Killer T (NKT) cells, macrophages, dendritic cells, B-cells, innate lymphoid cells (ILC), cytokine induced killer (CIK) cells, cytotoxic T lymphocytes (CTL), lymphokine activated killer (LAK) cells, or a mixture thereof.

The immune cells described herein may be engineered to express the engineered receptors disclosed herein. These cells are preferably obtained from the subject to be treated (i.e., are autologous). However, in some embodiments, immune cell lines or donor immune cells (allogeneic) are used. The cells may be obtained from an individual directly or may be obtained from a depository or other storage facility. The cells may be from an individual in need of therapy for a medical condition, and following their manipulation to express the antigen-targeting CAR (using standard techniques for transduction and expansion for adoptive cell therapy, for example), they may be provided back to the individual from which they were originally sourced. In some cases, the cells are stored for later use for the individual or another individual.

Immune cells to be manipulated can be obtained from a number of sources, including peripheral blood mononuclear cells, bone marrow, lymph node tissue, cord blood, thymus tissue, tissue from a site of infection, ascites, pleural effusion, spleen tissue, and tumors. Immune cells can be obtained from blood collected from a subject using any number of techniques known to the skilled artisan, such as FICOLL™ separation. For example, cells from the circulating blood of an individual may be obtained by apheresis. In some embodiments, immune cells are isolated from peripheral blood lymphocytes by lysing the red blood cells and depleting the monocytes, for example, by centrifugation through a PERCOLL™ gradient or by counterflow centrifugal elutriation.

A specific subpopulation of immune cells can be further isolated by positive or negative selection techniques. For example, immune cells can be isolated using a combination of antibodies directed to surface markers unique to the positively selected cells, e.g., by incubation with antibody-conjugated beads for a time period sufficient for positive selection of the desired immune cells. Alternatively, enrichment of immune cell populations can be accomplished by negative selection using a combination of antibodies directed to surface markers unique to the negatively selected cells.

The immune cells may be comprised in a population of cells, and that population may have a majority that are manipulated to express one or more antigen-targeting receptors. A cell population may comprise at least, at most, or about 50 to 100% of immune cells that are manipulated to express one or more antigen-targeting receptors. A cell population may comprise 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of immune cells that are manipulated to express one or more antigen-targeting receptors. The one or more antigen-targeting receptors may be separate polypeptides that may or may not be encoded by one or more vectors.

The genetically modified immune cells expressing one or more antigen-targeting receptors may also comprise a population of cells, and the population of genetically modified immune cells may further comprise a subset of cells. In some embodiments, a subset of a population of genetically engineered immune cells comprises from 50 to 99% of the population of genetically engineered immune cells. A subset of a cell population may comprise 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the cells of the population. In some embodiments, subset of cells in a population of genetically engineered immune cells comprises 50% of the cells of the genetically engineered immune cell population. In some embodiments, subset of cells in a population of genetically engineered immune cells comprises 55% of the cells of the genetically engineered immune cell population. In some embodiments, subset of cells in a population of genetically engineered immune cells comprises 60% of the cells of the genetically engineered immune cell population. In some embodiments, subset of cells in a population of genetically engineered immune cells comprises 65% of the cells of the genetically engineered immune cell population. In some embodiments, subset of cells in a population of genetically engineered immune cells comprises 70% of the cells of the genetically engineered immune cell population. In some embodiments, subset of cells in a population of genetically engineered immune cells comprises 75% of the cells of the genetically engineered immune cell population. In some embodiments, subset of cells in a population of genetically engineered immune cells comprises 80% of the cells of the genetically engineered immune cell population. In some embodiments, subset of cells in a population of genetically engineered immune cells comprises 85% of the cells of the genetically engineered immune cell population. In some embodiments, subset of cells in a population of genetically engineered immune cells comprises 90% of the cells of the genetically engineered immune cell population. In some embodiments, subset of cells in a population of genetically engineered immune cells comprises 95% of the cells of the genetically engineered immune cell population.

Following genetic manipulation to express the one or more antigen-targeting receptors, the immune cells may be immediately infused or may be stored. In certain aspects, following genetic modification, the cells may be propagated for days, weeks, or months ex vivo as a bulk population within about 1, 2, 3, 4, 5 days or more following gene transfer into cells. In a further aspect, the transfectants or transductants are cloned and a clone demonstrating presence of a single integrated or episomally maintained expression cassette or plasmid, and expression of the antigen-targeting CAR is expanded ex vivo. The clone selected for expansion demonstrates the capacity to specifically recognize and lyse target antigen-expressing target cells. The recombinant immune cells may be expanded by stimulation with IL-2, or other cytokines that bind the common gamma-chain (e.g., IL-7, IL-12, IL-15, IL-21, and others). The recombinant immune cells may be expanded by stimulation with artificial antigen presenting cells.

In a further aspect, the immune cells and/or genetically modified immune cells may be cryopreserved. The immune cells and/or genetically modified immune cells may be cryopreserved after expansion of the immune cells and/or genetically modified immune cells in culture. The cells may be in a solution or medium comprising dextrose, one or more electrolytes, albumin, dextran, and DMSO. The solution may be sterile, nonpyogenic, and isotonic.

The immune cells may be manipulated to express the one or more antigen-targeting receptors to produce genetically modified immune cells for the intent of being modular with respect to a specific purpose. For example, cells may be generated, including for commercial distribution, expressing antigen-targeting CARs and/or TCRs (or distributed with a nucleic acid that encodes the mutant for subsequent transduction), and a user may modify them to express one or more other genes of interest (including therapeutic genes) dependent upon their intended purpose(s). For instance, an individual interested in treating target antigen-positive cells, including target antigen-positive cancer, may obtain or generate suicide gene-expressing cells (or heterologous cytokine-expressing cells) and modify them to express a receptor comprising a target antigen-specific scFv, or vice versa.

Embodiments of the disclosure encompass immune cells that express one or more antigen-targeting CARs and/or TCRs. The immune cell comprises a recombinant nucleic acid that encodes one or more antigen-targeting CARs and/or TCRs, in specific embodiments. In particular embodiments, the genome of the manipulated immune cells expressing the one or more antigen-targeting CARs and/or TCRs may not be modified, for example, by inhibiting one or more genes endogenous to the genome. In particular embodiments, the genome of the manipulated immune cells expressing the one or more antigen-targeting CARs and/or TCRs may be modified in any manner, but in specific embodiments the genome is modified by CRISPR gene editing, for example. The genome of the cells may be modified to enhance effectiveness of the cells for any purpose.

### A. T-Cells

In some embodiments, the immune cells to be manipulated to expressed one or more antigen-targeting receptors, thereby producing genetically engineered immune cells, are human T-cells. A T-cell is a type of lymphocyte. T-cells can be easily distinguished from other lymphocytes by the presence of a T-cell receptor (TCR) on their cell surface. A critical step in T-cell maturation is making a functional T-cell receptor (TCR). Each mature T-cell will ultimately contain a unique TCR that reacts to a random pattern, allowing the immune system to recognize many different types of pathogens. The TCR consists of two major components, the alpha and beta chains, containing random elements designed to produce a wide variety of different TCRs.

T-cells are derived from c-kit+ Sca1+ hematopoietic stem cells (HSCs), found in the bone marrow. The HSCs then differentiate into multipotent progenitors (MPPs) that retain the potential to become both myeloid and lymphoid cells. The process of differentiation then proceeds to a common lymphoid progenitor (CLP), which can only differentiate into T, B or NK cells. These CLP cells then migrate via the blood to the thymus, where they engraft. The earliest cells which arrived in the thymus are termed double-negative, as they express neither the CD4 nor CD8 co-receptor. The newly arrived CLP cells are CD4−CD8−CD44+CD25−ckit+ cells, and are termed early thymic progenitor (ETP) cells. These cells will then undergo a round of division and downregulate c-kit and are termed DN1 cells.

At the DN2 stage (CD44+CD25+), cells upregulate the recombination genes RAG1 and RAG2 and re-arrange the TCRβ locus, combining V-D-J and constant region genes in an attempt to create a functional TCRB chain. As the developing thymocyte progresses through to the DN3 stage (CD44−CD25+), the T-cell expresses an invariant a-chain called pre-Ta alongside the TCRB gene. If the rearranged β-chain successfully pairs with the invariant a-chain, signals are produced which cease rearrangement of the β-chain (and silences the alternate allele). Although these signals require this pre-TCR at the cell surface, they are independent of ligand binding to the pre-TCR. If the pre-TCR forms, then the cell downregulates CD25 and is termed a DN4 cell (CD25−CD44−). These cells then undergo a round of proliferation and begin to re-arrange the TCRα locus.

Double-positive thymocytes (CD4+/CD8+) migrate deep into the thymic cortex, where they are presented with self-antigens. These self-antigens are expressed by thymic cortical epithelial cells on MHC molecules on the surface of cortical epithelial cells. Only those thymocytes that interact with MHC-I or MHC-II will receive a survival signal, and thymocytes that do not interact (or do not interact strongly enough) do not receive a survival signal and die. Double-positive cells (CD4+/CD8+) that interact well with MHC class II molecules will eventually become CD4+ cells, whereas thymocytes that interact well with MHC class I molecules mature into CD8+ cells. A T-cell becomes a CD4+ cell by down-regulating expression of its CD8 cell surface receptors. If the cell does not lose its signal, it will continue downregulating CD8 and become a CD4+, single positive cell.

T-cells are grouped into two groups, conventional adaptive T-cells or innate-like T-cells, based on their function. CD4 and CD8 T-cells selected in the thymus undergo further differentiation in the periphery to specialized cells which have different functions. Conventional adaptive T-cells include cytotoxic T-cells, helper T-cells, memory T-cells, and regulatory T-cells. Innate-like T-cells include natural killer T-cells, mucosal associated invariant T-cells, and gamma delta T-cells.

T helper cells (TH cells) assist other lymphocytes, including maturation of B-cells into plasma cells and memory B-cells, and activation of cytotoxic T-cells and macrophages. These cells are also known as CD4+ T-cells as they express the CD4 on their surfaces. Helper T-cells become activated when they are presented with peptide antigens by MHC class II molecules, which are expressed on the surface of antigen-presenting cells (APCs). Once activated, they divide rapidly and secrete cytokines that regulate or assist the immune response. These cells can differentiate into one of several subtypes, which have different roles. Cytokines direct T-cells into particular subtypes.

CD8+ T-cells (T_{C} cells, CTLs, T-killer cells, killer T-cells) are cytotoxic, meaning they are able to directly kill virus-infected cells and cancer cells, for example. These cells are defined by the expression of the CD8 protein on their cell surface. Cytotoxic T-cells recognize their targets by binding to short peptides (8-11 amino acids in length) associated with MHC class I molecules, present on the surface of all nucleated cells. Cytotoxic T-cells also produce the key cytokines IL-2 and IFNγ. These cytokines influence the effector functions of other cells, in particular macrophages and NK cells.

One function of T-cells is immune-mediated cell death, and it is carried out by CD8+ cytotoxic T-cells and CD4+ helper T-cells. Unlike CD8+ killer T-cells, CD4+ helper T-cells function by indirectly killing cells identified as foreign by determining if and how other parts of the immune system respond to a specific, perceived threat to the immune system. Helper T-cells also use cytokine signaling to influence regulatory B-cells directly, and other cell populations indirectly.

Antigen-naive T-cells expand and differentiate into memory and effector T-cells after they encounter their cognate antigen within the context of an MHC molecule on the surface of an antigen presenting cell. Appropriate co-stimulation must be present at the time of antigen encounter for this process to occur. Memory T-cells include effector, central, tissue-resident memory T (Trm) cells, stem memory TSCM cells, and virtual memory T-cells. The single unifying theme for all memory T-cell subtypes is that they are long-lived and can quickly expand to large numbers of effector T-cells upon re-exposure to their cognate antigen. By this mechanism, memory T-cells provide the immune system with memory against previously encountered pathogens. Memory T-cells may be either CD4+ or CD8+ and usually express CD45RO.

Regulatory T-cells (T_{regs}) provide tolerance, whereby immune cells are able to distinguish invading cells from “self,” which prevents immune cells from inappropriately reacting against a subjects' own cells, known as an autoimmune response. For this reason, regulatory T-cells have also been called suppressor T-cells. Two major classes of CD4+ Treg cells have been described, FOXP3+ T_{reg} cells and FOXP3− T_{reg} cells. FOXP3+ T_{reg} cells can develop either during normal development in the thymus, and are then known as thymic Treg cells, or can be induced peripherally and are called peripherally derived Treg cells. FOXP3-T_{reg} cells include T_{reg} 17 cells, Tr1 cells, and Th3 cells, which are thought to originate during an immune response and act by producing suppressive molecules. Tr1 cells are associated with IL-10, and Th3 cells are associated with TGF-beta.

Natural killer T-cells bridge the adaptive immune system with the innate immune system. Unlike conventional T-cells that recognize protein peptide antigens presented by major histocompatibility complex (MHC) molecules, NKT-cells recognize glycolipid antigens presented by CD1d. Once activated, these cells can perform functions ascribed to both helper and cytotoxic T-cells: cytokine production and release of cytolytic/cell killing molecules. They are also able to recognize and eliminate some tumor cells and cells infected with herpes viruses.

Mucosal associated invariant T-cell (MAIT) cells display innate, effector-like qualities. In humans, MAIT-cells are found in the blood, liver, lungs, and mucosa, defending against microbial activity and infection. The MHC class I-like protein, MR1, is responsible for presenting bacterially-produced vitamin B metabolites to MAIT-cells. After the presentation of foreign antigen by MR1, MAIT-cells secrete pro-inflammatory cytokines and are capable of lysing bacterially-infected cells. MAIT-cells can also be activated through MR1-independent signaling. In addition to possessing innate-like functions, this T-cell subset supports the adaptive immune response and has a memory-like phenotype.

Gamma delta T-cells (γδ T-cells) represent a small subset of T-cells which possess a γδ TCR rather than the αβ TCR on the cell surface. Gamma delta T-cells are found mostly in the gut mucosa, within a population of intraepithelial lymphocytes. Gamma delta T-cells are not MHC-restricted and seem to be able to recognize whole proteins rather than requiring peptides to be presented by MHC molecules on APCs. Human γδ T-cells that use the Vγ9 and Vδ2 gene fragments constitute the major γδ T-cell population in peripheral blood and are unique in that they specifically and rapidly respond to a set of nonpeptidic phosphorylated isoprenoid precursors, collectively named phosphoantigens, which are produced by virtually all living cells. The most common phosphoantigens from animal and human cells (including cancer cells) are isopentenyl pyrophosphate (IPP) and its isomer dimethylallyl pyrophosphate (DMPP). Many microbes produce the highly active compound hydroxy-DMAPP (HMB-PP) and corresponding mononucleotide conjugates, in addition to IPP and DMAPP. Plant cells produce both types of phosphoantigens.

In certain embodiments, T-cells are obtained from peripheral blood mononuclear cells (PBMCs) commonly obtained by a leukapheresis process, unstimulated leukapheresis products (PBSC), human embryonic stem cells (hESCs), induced pluripotent stem cells (iPSCs), bone marrow, or umbilical cord blood, or T-cell lines by methods well known in the art. In some cases utilizing a leukapheresis process, collected apheresis products can be processed in various ways depending on the downstream procedures. Devices such as HAEMONETICS® Cell Saver 5+, COBE 2991, and Fresenius Kabi LOVO have the ability to remove gross red blood cells and platelet contaminants. Terumo ELUTRA® and Biosafe SEPAX® systems provide size-based cell fractionation for the depletion of monocytes and the isolation of lymphocytes. Instruments such as CLINIMACS® Plus and Prodigy systems allow the enrichment of specific subsets of T-cells, such as CD4+, CD8+, CD25+, or CD62L+ T-cells using Miltenyi beads post-cell washing.

The expansion of T-cells in culture requires sustained and adequate activation. T-cell activation needs a primary specific signal via the T-cell receptor and costimulatory signals such as CD28, 4-1BB, or OX40. T-cell activation is also required for the manipulation of T-cells to express one or more antigen-targeting receptors. Methods of activating T-cells include but are not limited to use of plate-bound anti-CD3 and anti-CD28 antibodies, use of antigen-presenting cells, or use of T-cell activation reagents, for example.

Antigen-presenting cells, such as dendritic cells (DCs), are the endogenous activators of T-cell responses. Another cell-based T-cell activation approach is through artificial antigen-presenting cells (AAPCs). Irradiated K562-derived AAPCs have been used to stimulate the expansion of CAR-T-cells. In some cases, immune cells are expanded in the presence of an effective amount of universal antigen presenting cells (UAPCs), including in any suitable ratio. The cells may be cultured with the UAPCs at a ratio of 10:1 to 1:10; 9:1 to 1:9; 8:1 to 1:8; 7:1 to 1:7; 6:1 to 1:6; 5:1 to 1:5; 4:1 to 1:4; 3:1 to 1:3; 2:1 to 1:2; or 1:1, including at a ratio of 1:2, for example.

Several off-the-shelf clinical-grade T-cell activation reagents are also available, including the Invitrogen CTS DYNABEADS® CD3/28, the Miltenyi MACS® GMP EXPACT™ Treg beads, Miltenyi MACS® GMP TRANSACT™ CD3/28 beads, and the Juno Stage Expamer technology.

DYNABEADS® CD3/28 are uniform super-paramagnetic beads covalently coupled to CD3 and CD28 antibodies. These beads the selection and activation of T-cells in a single step when used in conjunction with the Dynal CLINEXVIVO™ MPC™ magnet. Miltenyi EXPACT™ beads are paramagnetic beads conjugated to CD3-biotin, CD28 and anti-biotin monoclonal antibodies. By using various beads to T-cell ratios, EXPACTIM Treg beads can be used to expand both regulatory T-cells and conventional lineage T-cells. Miltenyi MACS® GMP TRANSACT™ CD3/28 beads are polymeric nanomatrix conjugated to CD3 or to CD28 monoclonal antibodies. The Expamer technology from Juno Therapeutics utilizes a unique core Streptamer technology to isolate viral-specific lymphocytes. As a soluble and dissociable T-cell stimulation reagent, Expamer efficiently induces T-cell receptor (TCR) signaling and efficiently activates T-cells to support retroviral transduction and expansion.

The engagement of T-cell surface CD3 molecules with soluble anti-CD3 monoclonal antibodies also supports T-cell activation in the presence of IL-2. In some cases, the immune cells are expanded in the presence of IL-2, such as at a concentration of 10-500, 10-400, 10-300, 10-200, 10-100, 10-50, 100-500, 100-400, 100-300, 100-200, 200-500, 200-400, 200-300, 300-500, 300-400, or 400-500 U/mL.

### B. NK Cells

In some embodiments, the immune cells to be manipulated to expressed one or more antigen-targeting receptors, thereby producing genetically engineered immune cells, are human natural killer (NK) cells. NK cells, a lymphoid component of the innate immune system, are CD56+/CD3− large granular lymphocytes of the innate immune system that are involved in immune responses against viral infection or cells undergoing malignant transformation and that produce MHC-unrestricted cytotoxicity and secrete proinflammatory cytokines and chemokines. Unlike T lymphocytes, NK cells do not require antigen sensitization or presentation by major histocompatibility complex (MHC) class I/II molecules to recognize their targets. Instead, NK cells are a subpopulation of lymphocytes that have spontaneous cytotoxicity against a variety of tumor cells, virus-infected cells, and some normal cells in the bone marrow and thymus. NK cells differentiate and mature in the bone marrow, lymph nodes, spleen, tonsils, and thymus. NK cells can be detected by specific surface markers, such as CD16 and/or, CD56 in humans. NK cells do not express T-cell antigen receptors, the pan T marker CD3, or surface immunoglobulin B-cell receptors.

In certain embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood or NK cell lines by methods well known in the art. Particularly, umbilical CB may be used to derive NK cells. In certain aspects, the NK cells are isolated and expanded by the previously described method of ex vivo expansion of NK cells (Spanholtz et al., 2011; Shah et al., 2013). In this method, CB mononuclear cells are isolated by ficoll density gradient centrifugation and cultured in a bioreactor with IL-2 and artificial antigen presenting cells (aAPCs). After 7 days, the cell culture may be depleted of any cells expressing CD3 and re-cultured for an additional 7 days. The cells may again be CD3-depleted and characterized to determine the percentage of CD56⁺/CD3 ⁻ cells or NK cells. In other methods, umbilical CB is used to derive NK cells by the isolation of CD34⁺ cells and differentiation into CD56⁺/CD3 ⁻ cells by culturing in medium contain SCF, IL-7, IL-15, and/or IL-2.

### C. B-Cells

In some embodiments, the immune cells to be manipulated to expressed one or more antigen-targeting receptors, thereby producing genetically engineered immune cells, are human B-cells. B-cells are a type of white blood cell of the lymphocyte subtype and function in the humoral immunity component of the adaptive immune system. B-cells produce antibody molecules which may be either secreted or inserted into the plasma membrane where they serve as a part of B-cell receptors. When a naïve or memory B-cell is activated by an antigen, it proliferates and differentiates into an antibody-secreting effector cell, known as a plasmablast or plasma cell. Additionally, B-cells present antigens and secrete cytokines. B-cells express B-cell receptors (BCRs) on their cell membrane, which allow the B-cell to bind to a foreign antigen, against which it will initiate an antibody response.

B-cell types that may be manipulated to expressed one or more antigen-targeting receptors, thereby producing genetically engineered immune cells, include plasmablasts, plasma cells, lymphoplasmacytoid cells, memory B-cells, B-2 cells, and regulatory B-cells.

Plasmablasts are a short-lived, proliferating antibody-secreting cell arising from β cell differentiation. They are generated early in an infection and can result from T-cell-independent activation of B-cells or the extrafollicular response from T-cell-dependent activation of β cells.

Plasma cells are a long-lived, non-proliferating antibody-secreting cell arising from B-cell differentiation. They are generated later in an infection and, compared to plasmablasts, have antibodies with a higher affinity towards their target antigen due to affinity maturation in the germinal center (GC) and produce more antibodies. Plasma cells can result from the germinal center reaction from T-cell-dependent activation of B-cells, though they can also result from T-cell-independent activation of B-cells.

Lymphoplasmacytoid cells are cells with a mixture of B lymphocyte and plasma cell morphological features that are thought to be closely related to or a subtype of plasma cells.

Memory B-cells are dormant B-cells arising from B-cell differentiation. They may circulate through the body and initiate a stronger, more rapid antibody response if they detect the antigen that had activated their parent B-cell. Memory B cells can be generated from T-cell-dependent activation through both the extrafollicular response and the germinal center reaction as well as from T-cell-independent activation.

B-2 cells include Follicular (FO) B-cells and Marginal Zone (MZ) B-cells. FO B-cells are the most common type of B cell and, when not circulating through the blood, are found mainly in the lymphoid follicles of secondary lymphoid organs. They are responsible for generating the majority of high-affinity antibodies during an infection. MZ B-cells are found mainly in the marginal zone of the spleen and can serve as a first line of defense against blood-borne pathogens. B-2 cells can undergo both T-cell-independent and T-cell-dependent activation.

Regulatory B-cells (Bregs) are an immunosuppressive B-cell type that stop the expansion of pathogenic, pro-inflammatory lymphocytes through the secretion of, e.g., IL-10, IL-35, and TGF-β. Bregs can also promote the generation of Tregs by directly interacting with T-cells to skew their differentiation.

### D. Myeloid Cells

In some embodiments, the immune cells to be manipulated to expressed one or more antigen-targeting receptors, thereby producing genetically engineered immune cells, are human myeloid cells. Myeloid or myelogenous cell are blood cells that arise from a progenitor cell, and myeloid or myelogenous cell types that may be manipulated to expressed one or more antigen-targeting receptors, thereby producing genetically engineered immune cells, include granulocytes, monocytes, erythrocytes, and platelets.

Granulocytes are a category of leukocyte, or white blood cell, in the innate immune system characterized by the presence of specific granules in their cytoplasm. They are also called polymorphonuclear leukocytes (PMN, PML, or PMNL) because of the varying shape of the nucleus, which is usually lobed into three segments. Granulocytes include neutrophils, eosinophils, basophils, and mast cells and are produced via granulopoiesis in the bone marrow. Neutrophils constitute 60% to 65% of the total circulating white blood cells and consist of two subpopulations: neutrophil-killers and neutrophil-cagers. Neutrophils attack micro-organisms by phagocytosis, release of soluble anti-microbials (including granule proteins), and generation of neutrophil extracellular traps. Neutrophils can secrete products that stimulate monocytes and macrophages to increase phagocytosis and the formation of reactive oxygen compounds involved in intracellular killing. Eosinophils have a limited ability to participate in phagocytosis, they are professional antigen-presenting cells, they regulate other immune cell functions (e.g., CD4+ T cell, dendritic cell, B-cell, mast cell, neutrophil, and basophil functions), they are involved in the destruction of tumor cells, and they promote the repair of damaged tissue. Basophils release histamine and prostaglandins, which contribute to the inflammatory response that helps fight invading organisms by causing dilation and increased permeability of capillaries and allow blood-clotting elements and phagocytes to be delivered to infected areas. Mast cells mediate host defense against pathogens (e.g., parasites) and allergic reactions and are also involved in mediating inflammation and autoimmunity as well as mediating and regulating neuroimmune system responses.

Monocytes are also a type of leukocyte, or white blood cell. They are the largest type of leukocyte and can differentiate into macrophages and myeloid lineage dendritic cells. As a part of the vertebrate innate immune system, monocytes also influence the process of adaptive immunity. Monocytes compose 2% to 10% of all leukocytes in the human body and serve multiple roles in immune function. Such roles include: replenishing resident macrophages under normal conditions; migration within approximately 8-12 hours in response to inflammation signals from sites of infection in the tissues; and differentiation into macrophages or dendritic cells to effect an immune response. In an adult human, half of the monocytes are stored in the spleen. These change into macrophages after entering into appropriate tissue spaces, and can transform into foam cells in endothelium. There are at least three subclasses of monocytes in human blood based on their phenotypic receptors. The classical monocyte is characterized by high level expression of the CD14 cell surface receptor (CD14++ CD16− monocyte). The non-classical monocyte shows low level expression of CD14 and additional co-expression of the CD16 receptor (CD14+CD16++ monocyte). The intermediate monocyte shows high level expression of CD14 and low level expression of CD16 (CD14++CD16+ monocytes).

### IV. Methods of Producing Genetically Engineered Immune Cells

Disclosed herein, in some aspects, is a method of generating genetically engineered immune cells and/or a population of genetically engineered immune cells. The method can comprise (i) expanding immune cells, such as a population of immune cells, in culture with one or more TKIs; (ii) manipulating the immune cells to express one or more antigen-targeting receptors to produce the genetically engineered immune cells; and (iii) expanding the genetically modified immune cells in culture with one or more TKIs. In some cases, the genetically engineered immune cells express one or more target antigens to which the one or more antigen-targeting receptors specifically bind. In some cases, signaling by the one or more antigen-targeting receptors upon binding of the one or more target antigens expressed by the genetically engineered immune cells by the one or more antigen-targeting receptors of the genetically engineered immune cells is reduced upon culture of the immune cells and/or the genetically engineered immune cells in the presence of the one or more TKIs. In some cases, a reduction in signaling by the one or more antigen-targeting receptors upon binding of the one or more target antigens expressed by the genetically engineered immune cells by the one or more antigen-targeting receptors of the genetically engineered immune cells reduces immune cell activation, differentiation, and/or fratricide by the genetically engineered immune cells during expansion of the genetically engineered immune cells in culture compared to genetically engineered immune cells cultured in the absence of the one or more TKIs.

In some embodiments, the immune cells are activated as described elsewhere herein prior to expanding the population of immune cells in culture with the one or more TKIs.

Preparation methods of the disclosure may produce a population of genetically engineered immune cells comprising at least, at most, or about 102-1012 clonal cells. The method may produce a cell population comprising at least, at most, or about 102-1012 total cells, for example, at least, at most, or about 102, 103, 104, 105, 106, 107, 108, 109, 1010, 1011, 1012 total cells, or any range or value derivable therein. The produced cell population may be frozen and then thawed. In some cases of the preparation method, the method further comprises introducing one or more additional nucleic acids into the frozen and thawed cell population, such as the one or more additional nucleic acids encoding one or more therapeutic gene products, for example.

### A. Genetic Engineering of Immune Cells

Genetic modification may be introduced to immune cells to generate antigen- and/or ligand-specific immune cells (referred to herein in some cases as “genetically engineered,” “genetically modified,” or “engineered” immune cells). In specific embodiments, any composition may be delivered to recipient immune cells by any suitable methods. The compositions may be delivered to the cells by electroporation or by a vector, for example. In specific embodiments, for example, one or more compositions for introduction of at least one or more heterologous antigen receptors are delivered to the immune cells in a vector. In some embodiments, one or more compositions for gene editing are delivered to the cells in a vector encoding an antigen- and/or ligand-specific chimeric antigen receptor (CAR) or T-cell receptor (TCR) for the generation of antigen- and/or ligand-specific cells. One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g., derived from Moloney murine leukemia virus vectors (MoMLV), MSCV, SFFV, MPSV, SNV, etc.), lentiviral vectors (e.g., derived from HIV-1, HIV-2, SIV, BIV, FIV, etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

In specific embodiments, the vector is a multicistronic vector, such as is described in PCT/US19/62014, which is incorporated by reference herein in its entirety. In such cases, a single vector may encode the CAR or TCR (and the expression construct may be configured in a modular format to allow for interchanging parts of the CAR or TCR), a suicide gene, and one or more cytokines.

### 1. Viral Vectors

In specific embodiments, one or more isolated nucleic acids encoding one or more heterologous antigen receptors is introduced into immune cells using one or more recombinant expression vectors such as a viral vector including at least a lentivirus, a retrovirus, gamma-retroviruses, an adeno-associated virus (AAV), a herpesvirus, or adenovirus, for example.

Viral vectors encoding an antigen receptor may be provided in certain aspects of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present disclosure are described below.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

a. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

b. Promoter/Enhancers

The expression constructs provided herein comprise a promoter to drive expression of the antigen receptor. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the βlactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at GENBANK®, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

c. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Additionally, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the F2A (Foot-and-mouth disease virus 2A) or a “2A-like” sequence (e.g., Thosea asigna virus 2A; T2A).

d. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

e. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### 2. Other Methods of Nucleic Acid Delivery

In addition to viral delivery of the nucleic acids encoding the one or more antigen receptors, the following are additional methods of recombinant gene delivery to a given host cell and are thus considered in the present disclosure.

Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

### 3. Gene Editing and CRISPR

The immune cell production process of the disclosure may include gene editing of the immune cells to remove 1, 2, 3, 4, 5, 6, 7, 8, 9, 10, or more endogenous genes in the immune cells. In some cases the gene editing occurs in immune cells expressing one or more heterologous antigen receptors, whereas in other cases the gene editing occurs in immune cells that do not express a heterologous antigen receptor but that ultimately will express one or more heterologous antigen receptors, in at least some cases. In particular embodiments, the immune cells that are gene edited are expanded immune cells.

In other cases gene editing does not occur in immune cells expressing one or more heterologous antigen receptors or in immune cells that do not express a heterologous antigen receptor but that ultimately will express one or more heterologous antigen receptors, in at least some cases. In particular embodiments, expanded immune cells are not genetically edited.

In particular cases, one or more endogenous genes of the immune cells are modified, such as disrupted in expression where the expression is reduced in part or in full. In specific cases, one or more genes are knocked down or knocked out using processes of the disclosure. In specific cases, multiple genes are knocked down or knocked out in the same step as processes of the disclosure. The genes that are edited in the immune cells may be of any kind, but in specific embodiments the genes are genes whose gene products inhibit activity and/or proliferation of immune cells. In specific cases the genes that are edited in the immune cells allow the immune cells to work more effectively in a tumor microenvironment, including but not limited to, for example, PDCD1, TRAC, TRBC, b2M, and CIITA.

In some embodiments, the gene editing is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. The tracr sequence, which may comprise or consist of all or a portion of a wild-type tracr sequence (e.g., about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SWISSPROT® database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies, ELAND (ILLUMINA®, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US20110059502, incorporated herein by reference.

### B. Immune Cell Culture & Expansion

In certain aspects, starting immune cells of a selected population may comprise at least or about 10⁴, 10⁵, 10⁶, 10⁷, 10⁸, 10⁹, 10¹⁰, 10¹¹, 10¹², 10¹³ cells, or any range derivable therein. The starting cell population may have a seeding density of at least or about 10, 10¹, 10², 10³, 10⁴, 10⁵, 10⁶, 10⁷, 10⁸ cells/ml, or any range derivable therein.

A culture vessel used for culturing the 3D cell aggregates or progeny cells thereof can include, but is particularly not limited to: flask, flask for tissue culture, dish, petri dish, dish for tissue culture, multi dish, micro plate, micro-well plate, multi plate, multi-well plate, micro slide, chamber slide, tube, tray, CELLSTACK® Chambers, culture bag, and roller bottle, as long as it is capable of culturing the stem cells therein. The stem cells may be cultured in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, or any range derivable therein, depending on the needs of the culture. In a certain embodiment, the culture vessel may be a bioreactor, which may refer to any device or system that supports a biologically active environment. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

The culture vessel can be cellular adhesive or non-adhesive and selected depending on the purpose. The cellular adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used). The substrate for cell adhesion includes collagen, gelatin, poly-L-lysine, poly-D-lysine, laminin, and fibronectin and mixtures thereof for example MATRIGEL™, and lysed cell membrane preparations.

Various defined matrix components may be used in the culturing methods or compositions. For example, recombinant collagen IV, fibronectin, laminin, and vitronectin in combination may be used to coat a culturing surface as a means of providing a solid support for pluripotent cell growth, as described in Ludwig et al. (2006a; 2006b), which are incorporated by reference in its entirety.

A matrix composition may be immobilized on a surface to provide support for cells. The matrix composition may include one or more extracellular matrix (ECM) proteins and an aqueous solvent. The term “extracellular matrix” is recognized in the art. Its components include one or more of the following proteins: fibronectin, laminin, vitronectin, tenascin, entactin, thrombospondin, elastin, gelatin, collagen, fibrillin, merosin, anchorin, chondronectin, link protein, bone sialoprotein, osteocalcin, osteopontin, epinectin, hyaluronectin, undulin, epiligrin, and kalinin. Other extracellular matrix proteins are described in Kleinman et al., (1993), herein incorporated by reference. It is intended that the term “extracellular matrix” encompass a presently unknown extracellular matrix that may be discovered in the future, since its characterization as an extracellular matrix will be readily determinable by persons skilled in the art.

In some aspects, the total protein concentration in the matrix composition may be about 1 ng/mL to about 1 mg/mL. In some embodiments, the total protein concentration in the matrix composition is about 1 μg/mL to about 300 μg/mL. In more preferred embodiments, the total protein concentration in the matrix composition is about 5 μg/mL to about 200 μg/mL.

The extracellular matrix (ECM) proteins may be of natural origin and purified from human or animal tissues. Alternatively, the ECM proteins may be genetically engineered recombinant proteins or synthetic in nature. The ECM proteins may be a whole protein or in the form of peptide fragments, native or engineered. Examples of ECM protein that may be useful in the matrix for cell culture include laminin, collagen I, collagen IV, fibronectin and vitronectin. In some embodiments, the matrix composition includes synthetically generated peptide fragments of fibronectin or recombinant fibronectin.

In still further embodiments, the matrix composition includes a mixture of at least fibronectin and vitronectin. In some other embodiments, the matrix composition preferably includes laminin.

The matrix composition preferably includes a single type of extracellular matrix protein. In some embodiments, the matrix composition includes fibronectin, particularly for use with culturing progenitor cells. For example, a suitable matrix composition may be prepared by diluting human fibronectin, such as human fibronectin sold by Becton, Dickinson & Co. of Franklin Lakes, N.J. (BD®) (Cat#354008), in Dulbecco's phosphate buffered saline (DPBS) to a protein concentration of 5 μg/mL to about 200 μg/mL. In a particular example, the matrix composition includes a fibronectin fragment, such as RETRONECTIN®. RETRONECTIN® is a ˜63 kDa protein of (574 amino acids) that contains a central cell-binding domain (type III repeat), a high affinity heparin-binding domain II (type III repeat), and CS1 site within the alternatively spliced IIICS region of human fibronectin.

In some other embodiments, the matrix composition may include laminin. For example, a suitable matrix composition may be prepared by diluting laminin (SIGMA-ALDRICH® (St. Louis, Mo.); Cat#L6274 and L2020) in Dulbecco's phosphate buffered saline (DPBS) to a protein concentration of 5 μg/ml to about 200 μg/ml.

In some embodiments, the matrix composition is xeno-free, in that the matrix is or its component proteins are only of human origin. This may be desired for certain research applications. For example in the xeno-free matrix to culture human cells, matrix components of human origin may be used, wherein any non-human animal components may be excluded. In certain aspects, MATRIGEL™ may be excluded as a substrate from the culturing composition. MATRIGEL™ is a gelatinous protein mixture secreted by mouse tumor cells and is commercially available from BD® Biosciences (New Jersey, USA). This mixture resembles the complex extracellular environment found in many tissues and is used frequently by cell biologists as a substrate for cell culture, but it may introduce undesired xeno antigens or contaminants.

The immune cells and/or genetically engineered immune cells may be expanded using several expansion platforms readily available to generate therapeutic doses of genetically modified cells. The GE WAVE BIOREACTOR™ system is a widely used device for expansion. This scalable system consists of a single use CELLBAG™ Bioreactor, a temperature-enabling electric rocking base, and a range of optional controllers, pumps and probes. The CELLBAG™ Bioreactor is placed on a rocking base that is equipped to maintain bag inflation and gently rocks the cell bag for rapid gas transfer and mixing. The perfusion functionality of the WAVE BIOREACTOR™ allows for automatic feeding and waste removal. Cells can rapidly expand to more than 10⁷ cells/mL, and this system can support up to 25-L cell culture in a single bioreactor. The G-REX® platform is a cell culture flask with a gas-permeable membrane at the base that requires a low seeding density and allows cells to grow to a high density without compromising gas exchange. The Miltenyi CLINIMACS PRODIGY® system is a combination of a cell washer, the CLINIMACS® magnetic cell separation system, and a cell cultivation device. Finally, K562, a human leukemic cell line that does not express HLA class I A, HLA class I B nor HLA class II alleles, has been genetically modified to express a wide array of costimulatory molecules such as CD32, CD40, CD40L, CD64, CD70, CD80, CD83, CD86, CD137L, ICOSL, GITRL, CD134L, and membrane bound IL15 to facilitate T-cell expansion.

In particular embodiments, the immune cells, genetically engineered immune cells, and/or precursors thereto may be specifically formulated and/or they may be cultured in a particular medium at any stage of a process of generating the immune cells which express one or more of the genetically engineered receptors disclosed herein. The cells may be formulated in such a manner as to be suitable for delivery to a recipient without deleterious effects.

The medium used for culture and expansion of the cells in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include KNOCKOUT™ Serum Replacement (KSR) (THERMO FISHER SCIENTIFIC®, Chemically-defined Lipid Concentrate (GIBCO™), and GLUTAMAX™ (GIBCO™).

In further embodiments, the medium may be a serum-free medium that is suitable for cell development. For example, the medium may comprise B-27® supplement, xeno-free B-27® supplement (available at world wide web at thermofisher.com/us/en/home/technical-resources/media-formulation.250.html), NS21 supplement (Chen et al., J Neurosci Methods, 2008 Jun. 30; 171 (2): 239-247, incorporated herein in its entirety), GS21TM supplement (available at world wide web at amsbio.com/B-27.aspx), or a combination thereof at a concentration effective for producing T-cells from the 3D cell aggregate.

In particular embodiments, the immune cells, genetically engineered immune cells, and/or precursors thereto may be cultured in the presence of one or more tyrosine kinase inhibitors (TKIs). Mechanisms of intrinsic fratricide resistance that rely on antigen neutralization often produce undesirable ligand-driven CAR signaling that enhances T-cell differentiation to effector and effector memory populations. Specifically, CD3& chain signaling via Src kinases Lck and Fyn activates key signaling mediators, such as Itk, LAT, and PLCg, and triggers downstream signaling cascades. Signaling from the CD28 endodomain augments CD3ξ signaling by recruiting and activating Grb2, Lck, and Itk⁸. As this signaling network contributes to terminal T-cell differentiation, blocking these pathways during CAR T-cell manufacture would lead to cell products with a less differentiated phenotype, which is often desired in the adoptive cell therapy setting. In some embodiments, pharmacologic blockade of TKIs can prevent T-cell activation and degranulation during ex vivo expansion.

The one or more TKIs may comprise one or more Src kinase inhibitors. The one or more TKIs may comprise dasatinib, ibrutinib, pp2, pazopanib, gefitinib, or a combination thereof. In some embodiments, at least one of the one or more TKIs comprises dasatinib. In some embodiments, at least one of the one or more TKIs comprises ibrutinib. In some embodiments, the one or more TKIs comprise dasatinib and ibrutinib. In some cases, culturing the immune cells and/or the genetically engineered immune cells manipulated to express the one or more antigen-targeting receptors in the presence of one or more TKIs reduces signaling by the one or more antigen-targeting receptors upon binding of antigen(s) expressed by the genetically engineered immune cells. In some cases, a reduction in signaling by the one or more antigen-targeting receptors upon binding of the antigen(s) expressed by the genetically engineered immune cells by the one or more antigen-targeting receptors reduces immune cell activation, differentiation, and/or fratricide by the genetically engineered immune cells compared to genetically engineered immune cells cultured in the absence of the one or more TKIs. In some cases, culturing the immune cells and/or the genetically engineered immune cells manipulated to express the one or more antigen-targeting receptors in the presence of one or more TKIs reduces signaling by the one or more antigen-targeting receptors upon binding of antigen(s) acquired by the immune cells and/or the genetically engineered immune cells via trogocytosis and expressed by the genetically engineered immune cells by one or more antigen-targeting receptors. In some cases, a reduction in signaling by the one or more antigen-targeting receptors upon binding of the antigen(s) acquired via trogocytosis and expressed by the genetically engineered immune cells by the one or more antigen-targeting receptors reduces immune cell activation, differentiation, and/or fratricide by the genetically engineered immune cells compared to genetically engineered immune cells cultured in the absence of the one or more TKIs.

In some embodiments, the TKIs may be added to the culture of immune cells and/or genetically engineered immune cells at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein. In some embodiments, the concentration of each of the one or more TKIs in culture is between 0.01 μM to 10 μM. In some embodiments, the concentration of each of the one or more TKIs in culture is between 0.1 μM to 1 μM. In some embodiments, the concentration of each of the one or more TKIs is at least, at most, or about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 nM, μM, mM, or any range derivable therein.

In some embodiments, dasatinib is added to the culture of immune cells and/or genetically engineered immune cells at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein. In some embodiments, the concentration of dasatinib in culture is between 0.01 μM to 10 μM. In some embodiments, the concentration of dasatinib in culture is between 0.1 μM to 1 μM. In some embodiments, the concentration of dasatinib is at least, at most, or about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 nM, μM, mM, or any range derivable therein. In some embodiments, the concentration of dasatinib in culture is 0.5 μM.

In some embodiments, ibrutinib is added to the culture of immune cells and/or genetically engineered immune cells at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein. In some embodiments, the concentration of ibrutinib in culture is between 0.01 μM to 10 μM. In some embodiments, the concentration of ibrutinib in culture is between 0.1 μM to 1 μM. In some embodiments, the concentration of ibrutinib is at least, at most, or about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 nM, μM, mM, or any range derivable therein. In some embodiments, the concentration of ibrutinib in culture is 0.2 μM.

In some embodiments, the one or more TKIs are added to the culture of immune cells and/or genetically engineered immune cells between at least, at most, or about 0, 1, 2, 3, 4, 5, 6, or 7 days, or any range derivable therein, before manipulation of the immune cell population to express one or more antigen-targeting receptors. In some embodiments, the one or more TKIs are added to the culture between 0 to 7 days before manipulation of the immune cell population to express the one or more antigen-targeting receptors. In some embodiments, the one or more TKIs are added to the culture between 0 to 5 days before manipulation of the immune cell population to express the one or more antigen-targeting receptors. In some embodiments, the one or more TKIs are added to the culture between 0 to 3 days before manipulation of the immune cell population to express the one or more antigen-targeting receptors. In some embodiments, the one or more TKIs are added to the culture 7 days before manipulation of the immune cell population to express the one or more antigen-targeting receptors. In some embodiments, the one or more TKIs are added to the culture 6 days before manipulation of the immune cell population to express the one or more antigen-targeting receptors. In some embodiments, the one or more TKIs are added to the culture 5 days before manipulation of the immune cell population to express the one or more antigen-targeting receptors. In some embodiments, the one or more TKIs are added to the culture 4 days before manipulation of the immune cell population to express the one or more antigen-targeting receptors. In some embodiments, the one or more TKIs are added to the culture between 3 days before manipulation of the immune cell population to express the one or more antigen-targeting receptors. In some embodiments, the one or more TKIs are added to the culture between 2 days before manipulation of the immune cell population to express the one or more antigen-targeting receptors. In some embodiments, the one or more TKIs are added to the culture 1 day before manipulation of the immune cell population to express the one or more antigen-targeting receptors. In some embodiments, the one or more TKIs are added to the culture on the same day as manipulation of the immune cell population to express the one or more antigen-targeting receptors.

In some embodiments, the one or more TKIs are replenished in the culture of immune cells and/or genetically engineered immune cells at least, at most, or about every 0, 1, 2, 3, 4, 5, 6, or 7 days, or any range derivable therein, while the immune cells and/or genetically engineered immune cells are being cultured. In some embodiments, the one or more TKIs are replenished in the culture of immune cells and/or genetically engineered immune cells every day during culture. In some embodiments, the one or more TKIs are replenished in the culture of immune cells and/or genetically engineered immune cells every 2 days during culture. In some embodiments, the one or more TKIs are replenished in the culture of immune cells and/or genetically engineered immune cells every 3 days during culture. In some embodiments, the one or more TKIs are replenished in the culture of immune cells and/or genetically engineered immune cells every 4 days during culture. In some embodiments, the one or more TKIs are replenished in the culture of immune cells and/or genetically engineered immune cells every 5 days during culture. In some embodiments, the one or more TKIs are replenished in the culture of immune cells and/or genetically engineered immune cells every 6 days during culture. In some embodiments, the one or more TKIs are replenished in the culture of immune cells and/or genetically engineered immune cells every 7 days during culture.

In some embodiments, the one or more TKIs are depleted from the population of immune cells and/or genetically engineered immune cells after expanding the population of immune cells and/or genetically engineered immune cells for at least, at most, or about 0, 1, 2, 3, 4, 5, 6, 7, 8, 9, 10, 11, 12, 13, 14, 15, 16, 17, 18, 19, 20, or 21 days, or any range derivable therein, in culture. In some embodiments, the one or more TKIs are depleted from the population of immune cells and/or genetically engineered immune cells after expanding the population of immune cells and/or genetically engineered immune cells for 21 days in culture. In some embodiments, the one or more TKIs are depleted from the population of immune cells and/or genetically engineered immune cells after expanding the population of immune cells and/or genetically engineered immune cells for 14 days in culture. In some embodiments, the one or more TKIs are depleted from the population of immune cells and/or genetically engineered immune cells after expanding the population of immune cells and/or genetically engineered immune cells for 7 days in culture. In some embodiments, the one or more TKIs are depleted from the population of immune cells and/or genetically engineered immune cells after expanding the population of immune cells and/or genetically engineered immune cells for 6 days in culture. In some embodiments, the one or more TKIs are depleted from the population of immune cells and/or genetically engineered immune cells after expanding the population of immune cells and/or genetically engineered immune cells for 5 days in culture. In some embodiments, the one or more TKIs are depleted from the population of immune cells and/or genetically engineered immune cells after expanding the population of immune cells and/or genetically engineered immune cells for 4 days in culture. In some embodiments, the one or more TKIs are depleted from the population of immune cells and/or genetically engineered immune cells after expanding the population of immune cells and/or genetically engineered immune cells for 3 days in culture. In some embodiments, the one or more TKIs are depleted from the population of immune cells and/or genetically engineered immune cells after expanding the population of immune cells and/or genetically engineered immune cells for 2 days in culture. In some embodiments, the one or more TKIs are depleted from the population of immune cells and/or genetically engineered immune cells after expanding the population of immune cells and/or genetically engineered immune cells for 1 day in culture. In some embodiments, the expanded population of immune cells and/or genetically engineered immune cells is cryopreserved after the population of immune cells and/or genetically engineered immune cells is depleted of the one or more TKIs.

The population of immune cells and/or genetically engineered immune cells may be depleted of the one or more kinase inhibitors by sequential washes of the expanded populations of immune cells and/or genetically engineered immune cells with medium used for culture and expansion of the cells or medium in which the expanded cells will be stored. In some embodiments, at least, at most, or about 2, 3, 4, 5, or 6 sequential washes of the expanded populations of immune cells and/or genetically engineered immune cells are performed. In some embodiments, 2 sequential washes of the expanded populations of immune cells and/or genetically engineered immune cells are performed. In some embodiments, 3 sequential washes of the expanded populations of immune cells and/or genetically engineered immune cells are performed. In some embodiments, 4 sequential washes of the expanded populations of immune cells and/or genetically engineered immune cells are performed. In some embodiments, 5 sequential washes of the expanded populations of immune cells and/or genetically engineered immune cells are performed. In some embodiments, 6 sequential washes of the expanded populations of immune cells and/or genetically engineered immune cells are performed.

In certain embodiments, the medium may also comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine).

In some embodiments, the medium further comprises vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a B-27® supplement, xeno-free B-27® supplement, GS21TM supplement, or combinations thereof. In some embodiments, the medium comprises or further comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21TM supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese.

In further embodiments, the medium may comprise externally added ascorbic acid. The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts.

One or more of the additional medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

The medium used may be supplemented with at least one externally added cytokine at a concentration from about 0.1 ng/mL to about 500 ng/mL, more particularly 1 ng/ml to 100 ng/ml, or at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein. Suitable cytokines, include but are not limited to, FLT3 ligand (FLT3L), interleukin 7 (IL-7), stem cell factor (SCF), thrombopoietin (TPO), IL-2, IL-4, IL-6, IL-15, IL-21, TNF-alpha, TGF-beta, interferon-gamma, interferon-lambda, TSLP, thymopentin, pleotrophin, and/or midkine.

Other culturing conditions can be appropriately defined. For example, the culturing temperature can be about 20 to 40° C., such as at least, at most, or about 20, 21, 22, 23, 24, 25, 26, 27, 28, 29, 30, 31, 32, 33, 34, 35, 36, 37, 38, 39, 40° C. (or any range derivable therein), though the temperature may be above or below these values. The CO₂ concentration can be about 1, 2, 3, 4, 5, 6, 7, 8, 9, or 10% (or any range derivable therein), such as about 2% to 10%, for example, about 2 to 5%, or any range derivable therein. The oxygen tension can be at least or about 1, 5, 8, 10, 20%, or any range derivable therein.

### C. Immune Cell Selection

Isolation of immune cells before and/or after manipulation of the cells to express one or more antigen-targeting receptors include any selection methods, including cell sorters, magnetic separation using antibody-coated magnetic beads, packed columns; affinity chromatography; cytotoxic agents joined to a monoclonal antibody or used in conjunction with a monoclonal antibody, including but not limited to, complement and cytotoxins; and “panning” with antibody attached to a solid matrix, e.g., plate, or any other convenient technique.

The use of separation or isolation techniques include, but are not limited to, those based on differences in physical (density gradient centrifugation and counter-flow centrifugal elutriation), cell surface (lectin and antibody affinity), and vital staining properties (mitochondria-binding dye rho123 and DNA-binding dye Hoechst 33342). Techniques providing accurate separation include but are not limited to, FACS (Fluorescence-activated cell sorting) or MACS (Magnetic-activated cell sorting), which can have varying degrees of sophistication, e.g., a plurality of color channels, low angle and obtuse light scattering detecting channels, impedance channels, etc.

The antibodies utilized in the preceding techniques or techniques used to assess cell type purity (such as flow cytometry) can be conjugated to identifiable agents including, but not limited to, enzymes, magnetic beads, colloidal magnetic beads, haptens, fluorochromes, metal compounds, radioactive compounds, drugs or haptens. The enzymes that can be conjugated to the antibodies include, but are not limited to, alkaline phosphatase, peroxidase, urease and β-galactosidase. The fluorochromes that can be conjugated to the antibodies include, but are not limited to, fluorescein isothiocyanate, tetramethylrhodamine isothiocyanate, phycoerythrin, allophycocyanins and TEXAS RED™. For additional fluorochromes that can be conjugated to antibodies, see Haugland, Molecular Probes: Handbook of Fluorescent Probes and Research Chemicals (1992-1994). The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxygenin, oxazalone, and nitrophenol. The radioactive compounds that can be conjugated or incorporated into the antibodies are known to the art, and include but are not limited to technetium 99m (99TC), 125I and amino acids comprising any radionuclides, including, but not limited to, 14C, 3H and 35S.

Other techniques for positive selection may be employed, which permit accurate separation, such as affinity columns, and the like. The method should permit the removal to a residual amount of less than about 20%, preferably less than about 5%, of the non-target cell populations.

Cells may be selected based on light-scatter properties as well as their expression of various cell surface antigens. The purified stem cells have low side scatter and low to medium forward scatter profiles by FACS analysis. Cytospin preparations show the enriched stem cells to have a size between mature lymphoid cells and mature granulocytes.

Various techniques may be employed to separate the cells by initially removing cells of dedicated lineage. Monoclonal antibodies are particularly useful for identifying markers associated with particular cell lineages and/or stages of differentiation. The antibodies may be attached to a solid support to allow for crude separation. The separation techniques employed should maximize the retention of viability of the fraction to be collected. Various techniques of different efficacy may be employed to obtain “relatively crude” separations. Such separations are where up to 10%, usually not more than about 5%, preferably not more than about 1%, of the total cells present are undesired cells that remain with the cell population to be retained. The particular technique employed will depend upon efficiency of separation, associated cytotoxicity, ease and speed of performance, and necessity for sophisticated equipment and/or technical skill.

Selection of the progenitor cells need not be achieved solely with a marker specific for the cells. By using a combination of negative selection and positive selection, enriched cell populations can be obtained.

In certain embodiments, cells containing an exogenous nucleic acid may be identified in vitro or in vivo by including a marker in the expression vector or the exogenous nucleic acid, such as a selectable or screenable marker. Such markers would confer an identifiable change to the cell permitting easy identification of cells containing the expression vector. Generally, a selection marker may be one that confers a property that allows for selection. A positive selection marker may be one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

Selectable markers may include a type of reporter gene used in laboratory microbiology, molecular biology, and genetic engineering to indicate the success of a transfection or other procedure meant to introduce foreign DNA into a cell. Selectable markers are often antibiotic resistance genes; cells that have been subjected to a procedure to introduce foreign DNA are grown on a medium containing an antibiotic, and those cells that can grow have successfully taken up and expressed the introduced genetic material. Examples of selectable markers include: the Abicr gene or Neo gene from Tn5, which confers antibiotic resistance to geneticin.

A screenable marker may comprise a reporter gene, which allows the researcher to distinguish between wanted and unwanted cells. Certain embodiments of the present disclosure utilize reporter genes to indicate specific cell lineages. For example, the reporter gene can be located within expression elements and under the control of the ventricular- or atrial-selective regulatory elements normally associated with the coding region of a ventricular- or atrial-selective gene for simultaneous expression. A reporter allows the cells of a specific lineage to be isolated without placing them under drug or other selective pressures or otherwise risking cell viability.

Examples of such reporters include genes encoding cell surface proteins (e.g., CD4, HA epitope), fluorescent proteins, antigenic determinants and enzymes (e.g., β-galactosidase). The vector containing cells may be isolated, e.g., by FACS using fluorescently-tagged antibodies to the cell surface protein or substrates that can be converted to fluorescent products by a vector encoded enzyme.

In specific embodiments, the reporter gene is a fluorescent protein. A broad range of fluorescent protein genetic variants have been developed that feature fluorescence emission spectral profiles spanning almost the entire visible light spectrum. Mutagenesis efforts in the original Aequorea victoria jellyfish green fluorescent protein have resulted in new fluorescent probes that range in color from blue to yellow, and are some of the most widely used in vivo reporter molecules in biological research. Longer wavelength fluorescent proteins, emitting in the orange and red spectral regions, have been developed from the marine anemone, Discosoma striata, and reef corals belonging to the class Anthozoa. Still other species have been mined to produce similar proteins having cyan, green, yellow, orange, and deep red fluorescence emission. Developmental research efforts are ongoing to improve the brightness and stability of fluorescent proteins, thus improving their overall usefulness.

The cells in certain embodiments can be made to contain one or more genetic alterations by genetic engineering of the cells either before or after differentiation (US 2002/0168766). A cell is said to be “genetically altered”, “genetically modified” or “transgenic” when an exogenous nucleic acid or polynucleotide has been transferred into the cell by any suitable means of artificial manipulation, or where the cell is a progeny of the originally altered cell that has inherited the polynucleotide. For example, the cells can be processed to increase their replication potential by genetically altering the cells to express telomerase reverse transcriptase, either before or after they progress to restricted developmental lineage cells or terminally differentiated cells (U.S. Patent Application Publication 2003/0022367).

In embodiments wherein cells are genetically modified, such as to add or reduce one or more features, the genetic modification may occur by any suitable method. For example, any genetic modification compositions or methods may be used to introduce exogenous nucleic acids into cells or to edit the genomic DNA, such as gene editing, homologous recombination or non-homologous recombination, RNA-mediated genetic delivery or any conventional nucleic acid delivery methods. Non-limiting examples of the genetic modification methods may include gene editing methods such as by CRISPR/CAS9, zinc finger nuclease, or TALEN technology.

Genetic modification may also include the introduction of a selectable or screenable marker that aid selection or screening or imaging in vitro or in vivo. Particularly, in vivo imaging agents or suicide genes may be expressed exogenously or added to starting cells or progeny cells. In further aspects, the methods may involve image-guided adoptive cell therapy

### V. Methods of Treatment

In some embodiments, the immune cells produced by the methods of the disclosure are utilized for methods of treatment for an individual in need thereof. The immune cells of the disclosure may or may not be utilized directly after production. In some cases they are stored for later purpose. In any event, they may be utilized in therapeutic or preventative applications for a mammalian subject (human, dog, cat, horse, etc.) such as a patient. The individual may be in need of immune cell therapy for a medical condition of any kind, including cancer, infections of any kind, and/or any immune disorder, as examples. Methods may be employed with respect to individuals who have tested positive for a medical condition, who have one or more symptoms of a medical condition, or who are deemed to be at risk for developing such a condition.

Embodiments of the disclosure include methods of treating an individual for cancer, infections of any kind, and/or any immune disorder, as examples. In various embodiments, diseased or other cells expressing endogenous target antigen on their surface are targeted for the purpose of improving a medical condition including cancer, infections of any kind, and/or any immune disorder, as examples, in an individual that has the medical condition or for the purpose of reducing the risk or delaying the severity and/or onset of the medical condition in an individual. In some embodiments, the individual is one in which at least 5, 6, 7, 8, 9, 10, 11, 12, 13, 14, 15, 16, 17, 18, 19, 20, 21, 22, 23, 24, 25, 26, 27, 28, 39, or 30% of the diseased or other cells express the endogenous target antigen. In some embodiments, the patient is one that has been determined to have diseased cells that express the one or more target antigens. The individual may utilize the treatment method of the disclosure as an initial treatment or after (and/or with) another treatment.

In specific cases, cancer cells expressing endogenous target antigen are targeted for the purpose of killing the cancer cells. In cancer embodiments, the immunotherapy methods may be tailored to the need of an individual with cancer based on the type and/or stage of cancer, and in at least some cases the immunotherapy may be modified during the course of treatment for the individual.

Individuals treated with the present cell therapy may or may not have been treated for the particular medical condition prior to receiving the immune cell therapy. In some embodiments, the patient has received at least 1, 2, 3, 4, 5, 6, 7, 8, or more prior treatments for a cancer. The prior treatments may include a treatment or therapy described herein. In some embodiments, the prior treatments comprise conventional chemotherapy, conventional radiotherapy, conventional antiviral therapy, conventional antibacterial therapy, conventional immunosuppressive therapies, and the like. In some embodiments, the patient had received the prior therapy within 10, 20, 30, 40, 50, 60, 70, 80, or 90 days or hours of administration of the current compositions and cells of the disclosure. In some embodiments, the patient is one that has undergone prior therapy and has failed the prior treatment either because the prior treatment was not effective or because the prior treatment was deemed too toxic.

Antigen-targeting CAR and/or TCR constructs, nucleic acid sequences, vectors, immune cells, and so forth as contemplated herein, and/or pharmaceutical compositions comprising the same, that can be administered either alone or in any combination using standard vectors and/or gene delivery systems, and in at least some aspects, together with a pharmaceutically acceptable carrier or excipient, and that are used for the prevention, treatment or amelioration of immune disorders, solid cancers, hematologic cancers, and/or infectious disease infections. In particular embodiments, the pharmaceutical compositions of the present disclosure may be particularly useful in preventing, ameliorating and/or treating immune disorders, solid cancers, hematologic cancers, and/or infectious disease infections, including immune disorders, solid cancers, hematologic cancers, and/or infectious disease infections that express the target antigen.

In specific cases, examples of treatment methods are as follows: (1) adoptive cellular therapy with the produced immune cells (immune cells expanded in culture and expressing CARs or TCRs) to treat cancer patients with any type of hematologic malignancy, (2) adoptive cellular therapy with the produced immune cells (immune cells expanded in culture and expressing CARs or TCRs) to treat cancer patients with any type of solid cancers, (3) adoptive cellular therapy with the produced immune cells (immune cells expanded in culture and expressing CARs or TCRs) to treat patients with any type of infectious disease, and/or (4) adoptive cellular therapy with the produced immune cells (immune cells expanded in culture and expressing CARs or TCRs) to treat patients with any type of immune disorder.

In some embodiments, the present disclosure provides methods for immunotherapy comprising administering an effective amount of the immune cells produced by methods of the present disclosure. In one embodiment, a medical disease or disorder is treated by one or more transfers of immune cell populations produced by methods herein and that elicit an immune response, in at least particular cases. In certain embodiments of the present disclosure, cancer or infection is treated by delivery of one or more immune cell populations produced by methods of the disclosure and that elicits an immune response. Provided herein are methods for treating or delaying progression of cancer, immune disorders, and/or infectious diseases in an individual comprising administering to the individual an effective amount an antigen-specific immune cell therapy. The present methods may be applied for the treatment of immune disorders, solid cancers, hematologic cancers, and/or infectious disease infections.

Tumors for which the present treatment methods are useful include any malignant cell type, such as those found in a solid tumor or a hematological tumor. In cases wherein the individual has cancer, the cancer may be primary, metastatic, resistant to therapy, and so forth. In specific cases, the present therapy is useful for individuals with cancers that have been clinically indicated to be subject to immune cell regulation, including multiple types of solid tumors (melanoma, colon, lung, breast, and head and neck cancers), for example. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B-cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant: branchiolo-alveolar adenocarcinoma; papillary adenocarcinoma; chromophobe carcinoma: acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Particular embodiments concern methods of treatment of hematological malignancies, such as lymphoma or leukemia. Leukemia is a cancer of the blood or bone marrow and is characterized by an abnormal proliferation (production by multiplication) of blood cells, usually white blood cells (leukocytes). It is part of the broad group of diseases called hematological neoplasms. Leukemia is a broad term covering a spectrum of diseases. Leukemia is clinically and pathologically split into its acute and chronic forms.

Other embodiments concern methods of treatment of non-hematological malignancies, such as solid tumors including but not limited to tumors of an organ selected from the group consisting of pancreas, colon, cecum, stomach, brain, head, neck, ovary, kidney, larynx, sarcoma, lung, bladder, melanoma, prostate, and breast.

Certain embodiments of the present disclosure provide methods for treating or preventing an immune-mediated disorder. In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and immune cells are used to prevent and/or treat immune rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

Methods of treating an individual with a therapeutically effective amount of immune cells of the disclosure comprise administering the cells or clonal populations thereof to the patient. Thus, disclosed in some embodiments is a method of treating immune disorders, solid cancers, hematologic cancers, and/or infectious disease infections in a subject, the method comprising administering to a subject in need thereof a therapeutically effective amount of a composition comprising genetically engineered immune cells or a population of genetically engineered immune cells. In some embodiments, one or more antigens to which one or more antigen-targeting receptors specifically bind are expressed by the diseased cells in vivo, wherein the one or more CARs specifically bind the one or more antigens expressed by the diseased cells in vivo, and binding of the one or more antigen-targeting receptors to the one or more antigens expressed by the diseased cells in vivo results in elimination of the diseased cells.

In particular embodiments, the method is for treating cancer in a subject, and the method comprises administering to a subject in need thereof a therapeutically effective amount of a composition comprising genetically engineered immune cells or a population of genetically engineered immune cells. In some embodiments, one or more antigens to which one or more CARs and/or TCRs of the genetically engineered immune cells specifically bind are expressed by the cancer cells in vivo, wherein the one or more CARs and/or TCRs specifically bind the one or more antigens expressed by the cancer cells in vivo, and binding of the one or more CARs and/or TCRs to the one or more antigens expressed by the cancer cells in vivo results in elimination of the cancer cells.

In particular embodiments, the method is for treating hematological malignancies, such as T-cell malignancies, and the method comprises administering to a subject in need thereof a therapeutically effective amount of a composition comprising genetically engineered immune cells or a population of genetically engineered immune cells. In some embodiments, one or more antigens to which one or more CARs and/or TCRs of the genetically engineered immune cells specifically bind are expressed by the malignant T-cells in vivo, wherein the one or more CARs and/or TCRs specifically bind the one or more antigens expressed by the malignant T-cells in vivo, and binding of the one or more CARs and/or TCRs to the one or more antigens expressed by the malignant T-cells in vivo results in elimination of the malignant T-cells.

In particular embodiments, the method is for treating an immune disorder in a subject, and the method comprises administering to a subject in need thereof a therapeutically effective amount of a composition comprising genetically engineered immune cells or a population of genetically engineered immune cells. In some embodiments, one or more antigens to which one or more CARs and/or TCRs of the genetically engineered immune cells specifically bind are expressed by immune cells in vivo, wherein the one or more CARs and/or TCRs specifically bind the one or more antigens expressed by the immune cells in vivo, and binding of the one or more CARs and/or TCRs to the one or more antigens expressed by the immune cells in vivo results in elimination of the immune cells.

The cells or cell populations may be allogeneic with respect to the patient. The individual does not exhibit signs of depletion of the cells or cell population, in particular embodiments. In specific embodiments wherein the individual has cancer, tumor cells of the patient are killed after administering the cells or cell population or compositions thereof to the individual such that the cells contact the malignant tumor cells. In specific embodiments wherein the individual has an immune disorder, immune cells of the patient are killed after administering the cells or cell population or compositions thereof to the individual such that the cells contact the immune cells affected by the immune disorder.

In certain embodiments of the present disclosure, immune cells are delivered to an individual in need thereof, such as an individual that has cancer, immune disorder, or an infection. The cells then enhance the individual's immune system to attack the respective cancer or pathogenic cells. For individuals with cancer, once infused into the individuals it is expected that this cell product can employ multiple mechanisms to target and eradicate tumor cells. For individuals with an infectious disease, once infused into the individuals it is expected that this cell product can employ multiple mechanisms to target and eradicate infected cells. For individuals with an immune disorder, once infused into the individuals it is expected that this cell product can employ multiple mechanisms to target and eradicate cells affected by the immune disorder.

The one or more target antigens may comprise any fratricidal antigen. In some embodiments, the fratricidal antigen comprises CD1a, CD1b, CD1c, CD1d, CD1e, CD2, CD3d, CD3e, CD3g, CD4, CD5, CD6, CD7, CD8a, CD8b, CD9, CD10, CD11a, CD11b, CD11c, CD11d, CD13, CD14, CD15, CD16a, CD16b, CD17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD60b, CD60c, CD61, CD62E, CD62L, CD62P, CD63, CD64, CD65, CD66a, CD66b, CD66c, CD66d, CD66e, CD66f, CD67, CD68, CD69, CD70, CD71, CD72, CD73, CD74, CD75, CD75s, CD77, CD79a, CD79b, CD80, CD81, CD82, CD83, CD84, CD85a, CD85b, CD85c, CD85d, CD85e, CD85f, CD85g, CD85h, CD85i, CD85j, CD85k, CD86, CD87, CD88, CD89, CD90, CD91, CD92, CD93, CD94, CD95, CD96, CD97, CD98, CD99, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD122, CD123, CD124, CD125, CD126, CD127, CD128, CD129, CD130, CD131, CD132, CD133, CD134, CD135, CD136, CD137, CD138, CD139, CD140a, CD140b, CD141, CD142, CD143, CD144, CD146, CD147, CD148, CD150, CD151, CD152, CD153, CD154, CD155, CD156a, CD156b, CD156c, CD157, CD158a, CD158b1, CD158b2, CD158c, CD158d, CD158e, CD158f1, CD158f2, CD158g, CD158h, CD158i, CD158j, CD158k, CD158z, CD159a, CD159c, CD160, CD161, CD162, CD163, CD163b, CD164, CD165, CD166, CD167a, CD167b, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203a, CD203c, CD204, CD205, CD206, CD207, CD208, CD209, CD210, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD238, CD239, CD240CE, CD240D, CD241, CD242, CD243, CD244, CD245, CD246, CD247, CD248, CD249, CD252, CD253, CD254, CD256, CD257, CD258, CD261, CD262, CD263, CD265, CD266, CD267, CD268, CD269, CD270, CD271, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300b, CD300c, CD300d, CD300e, CD300f, CD300g, CD301, CD302, CD303, CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD360, CD361, CD362, or CD363.

In some embodiments, the one or more target antigens expressed by cancer cells, infected cells, and/or cells affected by the immune disorder comprise immune cell lineage antigens. In some embodiments, the immune cell lineage target antigen comprises CD2, CD5, CD7, CD4, CD8, CD3, CS1, CD38, CD99, CD30, 4-1BB, OX40, ICOS, CD26, CD6, TIGIT, PD-1, 2B4, LAG-3, MHC-I, MHC-II, peptide-MHC I, peptide-MHC II, Tim3, CTLA-4, CD112R, CD226, CD96, CD80, CD86, CD112, CD155, KIR2, KIR3, LILRB, CD28, CD40L, CD40, BTLA, GITR, VISTA, NKG2D ligands, or CD70. In some embodiments, the immune cell lineage target antigen comprises CD2. In some embodiments, the immune cell lineage target antigen comprises CD5. In some embodiments, the immune cell lineage target antigen comprises CD7. In some embodiments, the immune cell lineage target antigen comprises CD38.

In some embodiments, the one or more target antigens expressed by cancer cells, infected cells, and/or cells affected by the immune disorder comprise antigens acquired via trogocytosis. The target antigen may be associated with certain cancer cells, infected cells, and/or cells affected by the immune disorder but not associated with non-cancerous cells, non-infected cells, and/or cells unaffected by the immune disorder in some cases. The target antigen may be associated with both certain cancer cells and non-cancerous cells, certain infected cells and non-infected cells, and certain cells affected by the immune disorder and cells unaffected by the immune disorder, in some cases. The target antigen may comprise but is not limited to any target antigen disclosed herein. In some embodiments, the target antigen may be an antigen not normally expressed by immune cells that is artificially expressed by genetically manipulated immune cells to induce fratricide of the immune cells in vivo, thereby limiting the persistence and activity of the immune cells in vivo.

In specific embodiments, the dosing regimen is a single-dose of immune cell. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days, or 1, 2, 3, or 4 or more weeks, or 1, 2, 3, 4, 5, 6, 7, 8, 9, 10, 11, 12, or more months.

The immune cells may or may not be allogenic to the individual. Therapeutically effective amounts of the produced immune cells can be administered by a number of routes, including parenteral administration, for example, intravenous, intraperitoneal, intramuscular, intrasternal, intratumoral, intrathecal, intraventricular, through a reservoir, intraarticular injection, or infusion.

The therapeutically effective amount of the produced immune cells for use in adoptive cell therapy is that amount that achieves a desired effect in a subject being treated. For instance, this can be the amount of immune cells necessary to inhibit advancement, or to cause regression of cancer, or which is capable of relieving symptoms caused by cancer. This can be the amount of immune cells necessary to inhibit advancement, or to cause regression of an autoimmune or alloimmune disease, or which is capable of relieving symptoms caused by an autoimmune disease, such as pain and inflammation. It can also be the amount necessary to relieve symptoms associated with inflammation, such as pain, edema and elevated temperature. It can also be the amount necessary to diminish or prevent rejection of a transplanted organ.

The produced immune cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the type of disease to be treated, severity and course of the disease, the clinical condition of the individual, and/or the individual's clinical history and response to the treatment, and should be decided according to the judgment of the practitioner and each patient's circumstances. The therapeutically effective amount of immune cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ immune cells/m². In a certain embodiment, the dose used in the treatment of human subjects ranges from about 3.8×10⁹ to about 3.8×10¹⁰ immune cells/m². In additional embodiments, a therapeutically effective amount of immune cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. In additional embodiments, a therapeutically effective amount of immune cells can vary from about 10² up to about 10¹⁰ cells per kg of patient body weight whether by one or more administrations. In some embodiments, the therapy used is about 10² cells to about 10⁹ cells/kg of patient body weight, about 10² cells to about 10⁸ cells/kg of patient body weight, about 10² cells to about 10⁷ cells/kg of patient body weight, about 10² cells to about 10⁶ cells/kg of patient body weight, about 10² cells to about 10⁵ cells/kg of patient body weight, about 10² cells to about 10⁴ cells/kg of patient body weight, or about 10² cells to about 10³ cells/kg of patient body weight administered whether by one or more administrations, for example, once daily. In one embodiment, a therapy described herein is administered to a subject at a dose of about 10² cells, about 10³ cells, about 10⁴ cells, about 10⁵ cells, about 10⁶ cells, about 10⁷ cells, about 10⁸ cells, about 10⁹ cells, or about 10¹⁰ cells per kg of patient body weight. The exact amount of immune cells is readily determined by one of skill in the art based on the age, weight, sex, and physiological condition of the subject. Effective doses can be extrapolated from dose-response curves derived from in vitro or animal model test systems.

The immune cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B-cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

### A. Pharmaceutical Compositions

Also provided herein are pharmaceutical compositions and formulations comprising immune cells produced by the processes encompassed herein and a pharmaceutically acceptable carrier. The pharmaceutical compositions and formulations comprising immune cells disclosed herein may comprise administration of a combination of therapeutic agents, such as an immune cell therapeutic or pharmaceutical composition or treatment and one or more additional therapeutic or pharmaceutical compositions or treatments. The therapies may be administered in any suitable manner known in the art. For example, the therapies may be administered sequentially (at different times) or concurrently (at the same time). In some embodiments, the therapies are administered in a separate composition, for example, one separate composition, such as 2 separate compositions, 3 separate compositions, or 4 separate compositions. In some embodiments, the therapies are in the same composition.

Pharmaceutical compositions and formulations as described herein can be prepared by mixing the active ingredients (such as produced immune cells and one or more additional therapeutic agents) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGPs), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

The therapeutic or pharmaceutical compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic or pharmaceutical agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

In various embodiments the produced immune cells described herein can be administered either as a therapeutic or pharmaceutical composition alone, or as a therapeutic or pharmaceutical composition in combination with diluents and/or with other components such other cytokines or cell populations. Briefly, in certain embodiments, pharmaceutical compositions can comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Pharmaceutical or therapeutic compositions of the present disclosure may be administered in a manner appropriate to the disease to be treated (or prevented). The quantity and frequency of administration will be determined by such factors as the condition of the patient, and the type and severity of the subject's disease, although appropriate dosages may be determined by clinical trials. Precise amounts of the therapeutic or pharmaceutical composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing. When “an immunologically effective amount,” “an anti-tumor effective amount,” “an tumor-inhibiting effective amount,” or “therapeutic amount” is indicated, the precise amount of the compositions of the present disclosure to be administered can be determined by a physician with consideration of individual differences in age, weight, tumor size, extent of infection or metastasis, and condition of the patient (subject).

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of the therapeutic composition or treatment administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### B. Combination Therapies

In certain embodiments, the compositions and methods of the present embodiments involve an immune cell population in combination with at least one additional therapy. For cancer embodiments, the additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. For pathogenic conditions, the additional therapy may comprise one or more antibiotics, antivirals, and so forth.

In some cancer embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy of the disclosure may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### 1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gamma1I and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein transferase inhibitors, transplatinum, and pharmaceutically acceptable salts, acids, or derivatives of any of the above.

### 2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation, and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### 3. Immunotherapy

The skilled artisan will understand that additional immunotherapies may be used in combination or in conjunction with methods of the embodiments. In the context of cancer treatment, immunotherapeutics, generally, rely on the use of immune cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T-cells and NK cells.

Antibody-drug conjugates (ADCs) comprise monoclonal antibodies (MAbs) that are covalently linked to cell-killing drugs and may be used in combination therapies. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. Exemplary ADC drugs include ADCETRIS® (brentuximab vedotin) and KADCYLA® (trastuzumab emtansine or T-DM1).

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies include immune adjuvants, e.g., Mycobacterium bovis, Plasmodium falciparum, dinitrochlorobenzene, and aromatic compounds); cytokine therapy, e.g., interferons α, β, and γ, IL-1, GM-CSF, and TNF; gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T-cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies. Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody that may be used. Pembrolizumab, also known as MK-3475, Merck 3475, lambrolizumab, KEYTRUDA®, and SCH-900475, is an exemplary anti-PD-1 antibody. CT-011, also known as hBAT or hBAT-1, is also an anti-PD-1 antibody. AMP-224, also known as B7-DCIg, is a PDL2-Fc fusion soluble receptor.

Another immune checkpoint that can be targeted in the methods provided herein is the cytotoxic T-lymphocyte-associated protein 4 (CTLA-4), also known as CD152. The complete cDNA sequence of human CTLA-4 has the GENBANK® accession number L15006. CTLA-4 is found on the surface of T-cells and acts as an “off” switch when bound to CD80 or CD86 on the surface of antigen-presenting cells. CTLA4 is a member of the immunoglobulin superfamily that is expressed on the surface of Helper T-cells and transmits an inhibitory signal to T-cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T-cells, whereas CD28 transmits a stimulatory signal. Intracellular CTLA4 is also found in regulatory T-cells and may be important to their function. T-cell activation through the T-cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. An exemplary anti-CTLA-4 antibody is ipilimumab (also known as 10D1, MDX-010, MDX-101, and Yervoy®) or antigen binding fragments and variants thereof. In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

### 4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VI. Kits

Any of the compositions described herein may be comprised in a kit. In a non-limiting example, cells, reagents to produce cells, vectors, and reagents to produce vectors and/or components thereof may be comprised in a kit. In certain embodiments, immune cells may be comprised in a kit, and they may or may not yet express an antigen-targeting receptor. Such a kit may or may not have one or more reagents for manipulation of cells. Such reagents include small molecules, proteins, nucleic acids, antibodies, buffers, primers, nucleotides, salts, and/or a combination thereof, for example. Small molecules that may be used to manipulate the cells include tyrosine kinase inhibitors. Tyrosine kinase inhibitors including but not limited to dasatinib, ibrutinib, pp2, pazopanib, gefitinib, or a combination thereof, may be included in the kit. Nucleotides that encode one or more antigen-targeting CARs and/or TCRs, suicide gene products, and/or cytokines may be included in the kit. Proteins, such as cytokines or antibodies, including monoclonal antibodies, may be included in the kit. Nucleotides that encode components of engineered CARs and or TCRs may be included in the kit, including reagents to generate same.

In particular aspects, the kit comprises the immune cell therapy of the disclosure and also another cancer therapy. In some cases, the kit, in addition to the cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

The article of manufacture or kit can further comprise a package insert comprising instructions for using the immune cells to treat or delay progression of disease, for example, cancer, an infection, or an immune disorder, in an individual or to enhance immune function of an individual having cancer, an infection, or an immune disorder. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### EXAMPLES

The following examples are included to demonstrate embodiments of the disclosure. It should be appreciated by those of skill in the art that the techniques disclosed in the examples which follow represent techniques discovered by the inventor to function well in the practice of the disclosure. However, those of skill in the art should, in light of the present disclosure, appreciate that many changes can be made in the specific embodiments which are disclosed and still obtain a like or similar result without departing from the spirit and scope of the disclosure.

### Example 1

### Feasibility and Preclinical Efficacy of CD5 Car T-Cells for T-Cell Malignancies

The inventors developed second-generation CD5 CARs containing a single-chain variable fragment from a CD5 scFv antibody (see Mamonkin et al. Blood. 2015 Aug. 20; 126 (8): 983-992, and Vera et al. Blood. 2006 Dec. 1; 108 (12): 3890-3897, both incorporated by reference herein in their entirety). The remaining CAR backbone contains a C_{H}3 IgG1 Fc spacer with an IgG4-derived flexible hinge and CD28/CD35 signaling domains³⁸.

The inventors have shown that activated human T-cells transduced with a CD5 CAR can specifically recognize and kill malignant T-cell lines and primary T-ALL blasts³⁸. Although expansion of CD5 CAR T-cells was preceded by transient fratricide, the extent of self-killing was limited³⁸. The fratricide also induced the disappearance of CD5 from the cell surface and selected for a resistant differentiated population (38). CD5 CAR T-cells were also expanded in vitro where they recognized and eradicated CD5⁺ malignant T-cells and efficiently controlled disease progression in xenograft mouse models³⁸.

### 1. Selection of CAR Signaling Inhibitors

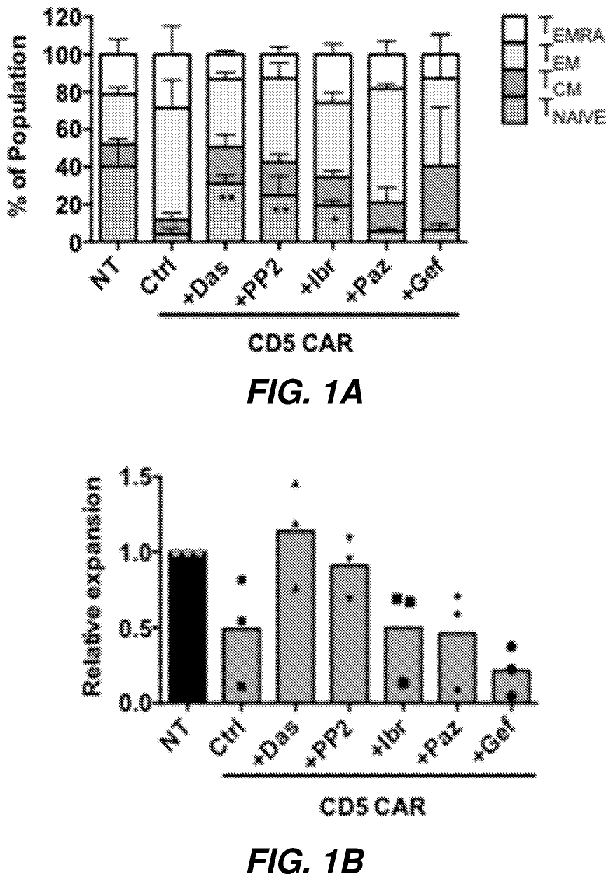

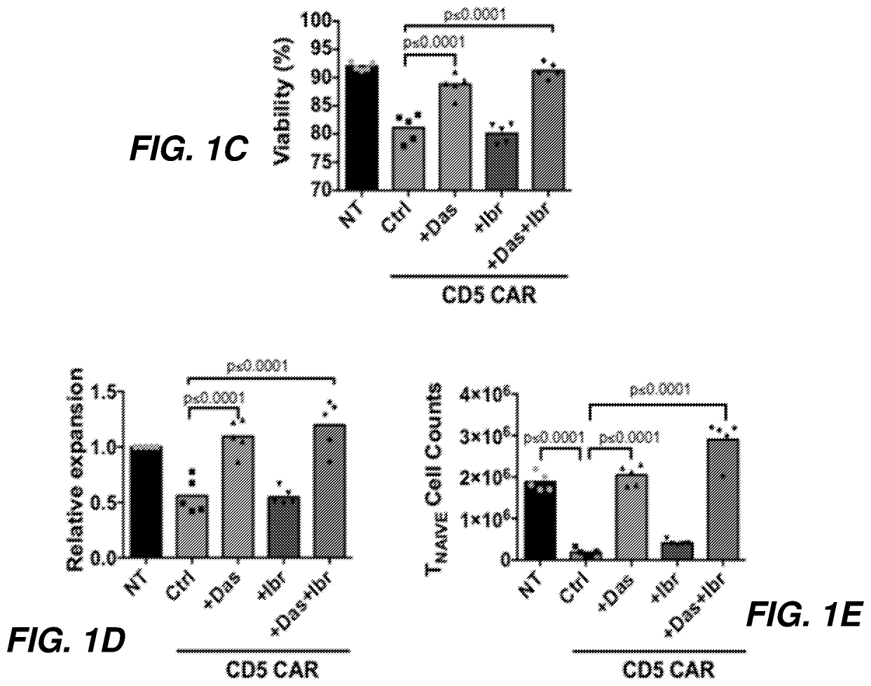

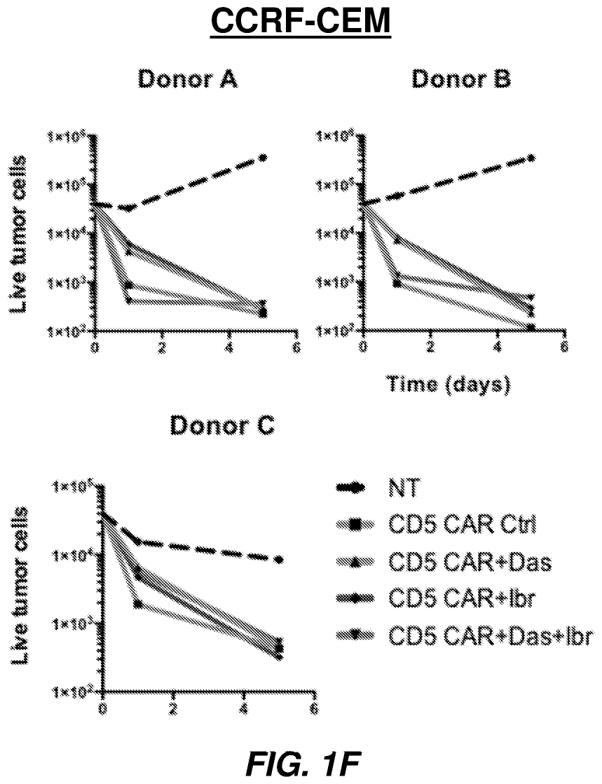

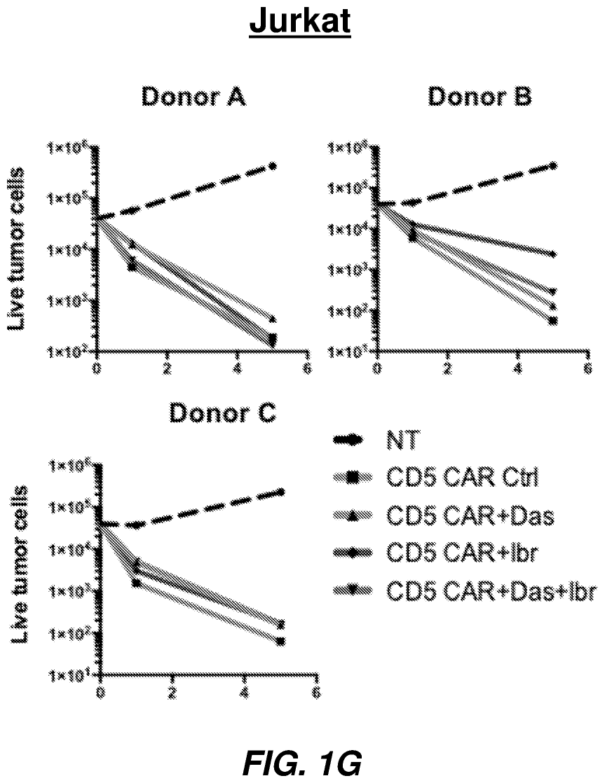

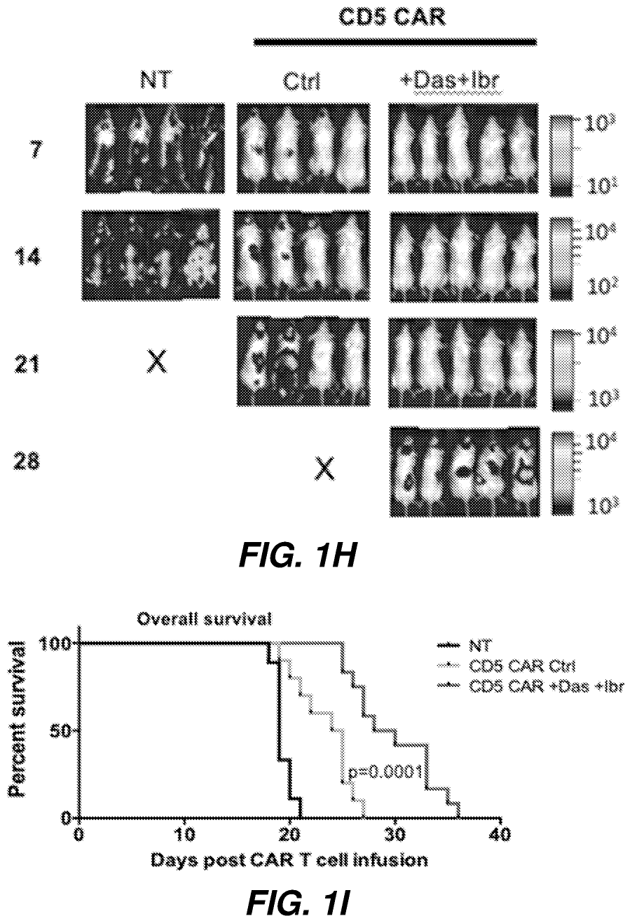

To select the most potent inhibitors of CAR signaling, the inventors expanded CD5 CAR T-cells in the presence of pharmacologic inhibitors of key proximal TCR signaling kinases Lck, ZAP-70, and Itk. On the day of viral transduction, the inventors added chemical inhibitors of Lck (dasatinib, pp2, pazopanib), ZAP-70 (gefitinib) and Itk (ibrutinib) to the T-cell conditioning medium at concentrations previously established to be effective and replenished them every 2-3 days. Addition of the chemical inhibitors did not impair gammaretroviral transduction, resulting in CD5 CAR expression in the majority of T-cells (data not shown). Continuous self-directed signaling from the CAR resulted in a significant depletion of minimally differentiated T-cell subsets of naïve-like and central memory cells in CD5 CAR T-cells (CD5 CAR Ctrl) compared to the control non-transduced T-cells (NT) (FIG. 1A). Subsequent expansion of CD5 CAR T-cells in the presence of dasatinib, pp2, or ibrutinib resulted in higher frequencies of minimally differentiated, naïve-like T-cells compared to control CD5 CAR T-cells expanded in the absence of inhibitors (FIG. 1A). Pazopanib and gefitinib had less impact on the differentiation of CD5 CAR T-cells. The chemical inhibitors did not inhibit CD5 CAR T-cell expansion, and both dasatinib and pp2 promoted robust CD5 CAR T-cell expansion, comparable to non-transduced control T-cells (FIG. 1B) indicating these inhibitors blocked CAR T-cell fratricide. No such effect was observed upon expansion of control non-transduced T-cells in the presence of the same inhibitors, indicating the effect is CAR-specific.

### 2. Pharmacologic Blockade of CAR Signaling Minimizes Fratricide of CD5 CAR T-Cells

The inventors evaluated whether individual or combinatorial blockade of Lck and Itk pathways minimized tonic CAR signaling in CD5 CAR T-cells and the associated differentiation and fratricide. For the combinatorial blockade of Lck and Itk, ibrutinib was administered during the initial T-cell priming at a lower dose (200 nM) and maintained at that concentration; dasatinib was added at a normal concentration (50 nM) on the day of CAR transduction. Addition of dasatinib alone or in combination with ibrutinib increased viability and overall expansion of CD5 CAR T-cells, as well as the overall frequency of naïve-like T-cells while ibrutinib alone had little effect (FIGS. 1C-1E). These data indicate a combination of dasatinib and ibrutinib effectively blocks CAR signaling. Chemical inhibition of CAR signaling must be reversible in order to allow CAR T-cells to regain cytotoxicity after removing inhibitors. To assess restoration of the anti-tumor activity upon removal of dasatinib and/or ibrutinib, the inventors expanded CD5 CAR T-cells in the presence of chemical inhibitors, cryopreserved, thawed, and resuspended in a normal conditioning medium. The inventors then co-cultured these CD5 CAR T-cells with CD5⁺ leukemia cell lines CCRF-CEM and Jurkat for 5 days. CD5 CAR T-cells expanded with dasatinib and/or ibrutinib controlled tumor at the end of the co-culture similarly to untreated control CD5 CAR T-cells (FIGS. 1F-1G).

3. Unedited CD5 CAR T-Cells Protect Mice from Systemic T-Cell Leukemia

To assess whether CD5 CAR T-cells expanded in the presence of dasatinib and ibrutinib can control leukemia progression in vivo, the inventors used a previously established mouse xenograft model of disseminated T-ALL. Briefly, NSG mice received FFLuc-modified CCRF-CEM cells intravenously followed by a single intravenous injection of freshly thawed CD5 CAR T-cells 3 days later. Expansion of CD5 CAR T-cells with dasatinib and ibrutinib had robust anti-leukemic activity (FIG. 1H), prolonging mouse survival compared to control CD5 CAR T-cells (FIG. 11). These results indicate the blockade of CD5 CAR signaling is reversible and allows rapid restoration CD5 CAR T-cell anti-tumor function in the absence of the chemical inhibitors.

### Example 2

### Feasibility and Preclinical Efficacy of CD7-Unedited CD7 Car T-Cells for T-Cell Malignancies

The inventors developed second-generation CD7 CARs containing a CD7 monoclonal antibody 3A1e^{34,38}. CD7-specific clone 3A1e originates from a murine hybridoma and has been developed for the therapy of T-cell malignancies as an antibody-drug conjugate DA 735.36, which has demonstrated safety and activity in a Phase I clinical study in patients with T-cell malignancies³⁷. The remaining CAR backbone contains a C_{H}3 IgG1 Fc spacer with an IgG4-derived flexible hinge and CD28/CD3ξ signaling domains³⁸.

CAR-mediated targeting of T-lineage antigens for the therapy of blood malignancies is frequently complicated by self-targeting of CAR T-cells or their excessive differentiation driven by constant CAR signaling. Expression of CARs targeting CD7, a pan-T cell antigen highly expressed in most T-cell acute lymphoblastic leukemias (T-ALL) and lymphomas, as well as in several subtypes of mature T-cell lymphomas, is an attractive target for cellular immunotherapy¹. However, high CD7 expression on normal T-cells causes substantial fratricide upon transduction with a CD7 CAR. Strategies have been developed to remove surface CD7 antigen through genome editing or an intracellular protein expression blocker (PEBL) 2 4. Both methods yield fratricide-resistant CD7 CAR T-cells that demonstrate high activity in preclinical models of CD7⁺ lymphoid and myeloid malignancies. Early clinical results indicate that these CD7 CAR T-cells can induce remissions in patients with recalcitrant T-cell malignancies but do not fully eliminate endogenous T-cells due to the presence of CD7-negative T-cell subsets resistant to CAR T-cell cytotoxicity⁵. The CD7-negative T-cell compartment represents a minority of circulating T-cells and contains both CD4⁺ and CD8⁺ T-cells, mainly from effector and memory compartments^{6,7}. The expression of a CD7 CAR on these CD7-negative T-cells is not expected to cause fratricide, thus potentially allowing manufacturing functional CD7 CAR T-cells without additional engineering. However, enriching the final T-cell product for CD7-negative CAR T-cells would either require additional cell sorting prior to CAR transduction or rely on CAR-mediated elimination of CD7−positive T-cells that could accelerate differentiation and exhaustion of CD7−negative CAR T-cells during ex vivo expansion and thus limit their therapeutic efficacy.

To validate the CD7−specific binder and evaluate the cytotoxic potential of the CD7 CAR, the inventors expressed it initially on CD7−edited T-cells to minimize unwanted self-directed activity. Expanded CD7 CAR T-cells were cytotoxic against a range of CD7⁺ T-ALL and T-cell lymphoma cell lines but produced no significant activity against a CD7-negative cell line NALM-6 (34). The inventors also detected robust production of TNFα and IFNγ by CD7 CAR T-cells upon co-culture with malignant T-cell lines³⁴. To assess the activity of CD7 CAR T-cells against primary T-cell tumors, the inventors measured cytokine production and residual live tumor cell counts after a brief co-culture. Similarly, co-culture of CD7 CAR T-cells with primary T-ALL tumor cells resulted in a significant production of cytokines and robust elimination of live tumor cells, correlating with the expansion of CAR T-cells³⁴. Overall, these results indicate the CD7 CAR elicits high cytotoxicity against CD7⁺ malignant T-cells.

To overcome the need for genome editing and streamline manufacturing of CD7 CAR T-cells and to overcome the limitations associated with enriching final T-cell product for CD7-negative CAR T-cells, the inventors developed a method to minimize fratricide of unedited CD7 CAR T-cells (which are overwhelmingly CD7⁺) using the FDA-approved pharmacologic inhibitors of key signaling kinases. It was tested whether CD7 CAR-mediated fratricide in T-cells can be temporarily minimized by blocking CAR signaling with tyrosine kinase inhibitors dasatinib and ibrutinib, which selectively inhibit key CAR/CD3ξ signaling kinases Lck and Itk, respectively. Src family kinases Lck and Fyn play a central role in initiating and propagating signaling from the CD3ξ chain, leading to the activation of downstream cascades via Itk. In addition, the CD28 costimulatory endodomain elicits Lck-dependent signaling and can directly recruit and activate Itk⁸.

In this study, supplementation with ibrutinib and dasatinib—pharmacologic inhibitors of Itk and Lck/Fyn, respectively—mitigated fratricide of CD7 CAR T-cells generated from whole peripheral blood T-cells and prevented terminal differentiation by reversibly blocking deleterious CAR signaling during ex vivo expansion. Anti-leukemic activity of these CD7 CAR T-cells upon removal of pharmacologic inhibitors was assessed and the mechanism by which the unedited CAR T-cells produced sustained anti-tumor activity in mouse xenograft models of human T-ALL was explored. Feasibility of cGMP manufacturing autologous unedited CD7 CAR T-cells for patients with CD7⁺ T-cell malignancies and initiated a Phase I clinical trial was also demonstrated. These results surprisingly demonstrate that pharmacologic inhibition of CAR signaling can enable generation of functional CD7 CAR T-cells without additional engineering.

### 1. Pharmacologic Inhibition of CAR Signaling Prevents Fratricide of CD7 CAR T-Cells

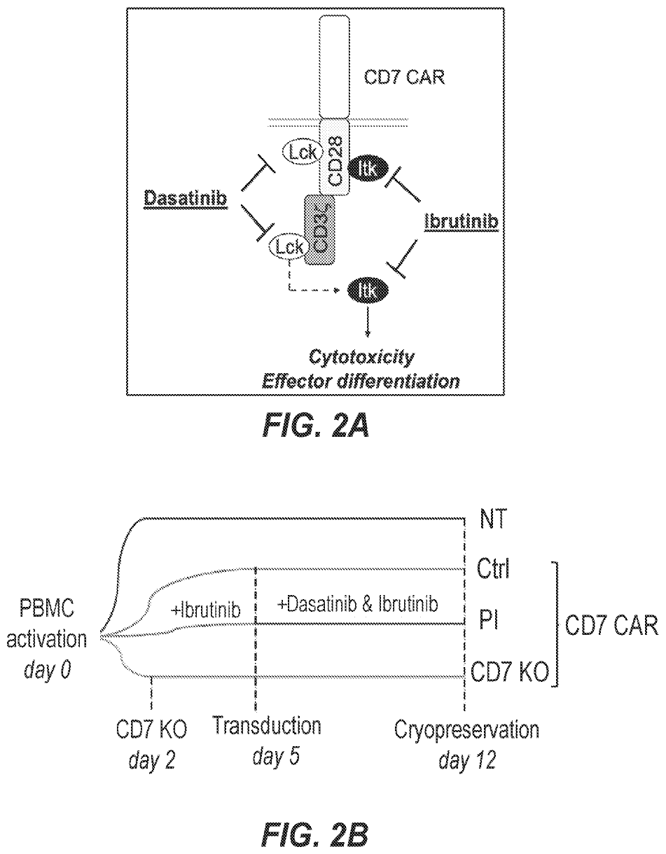

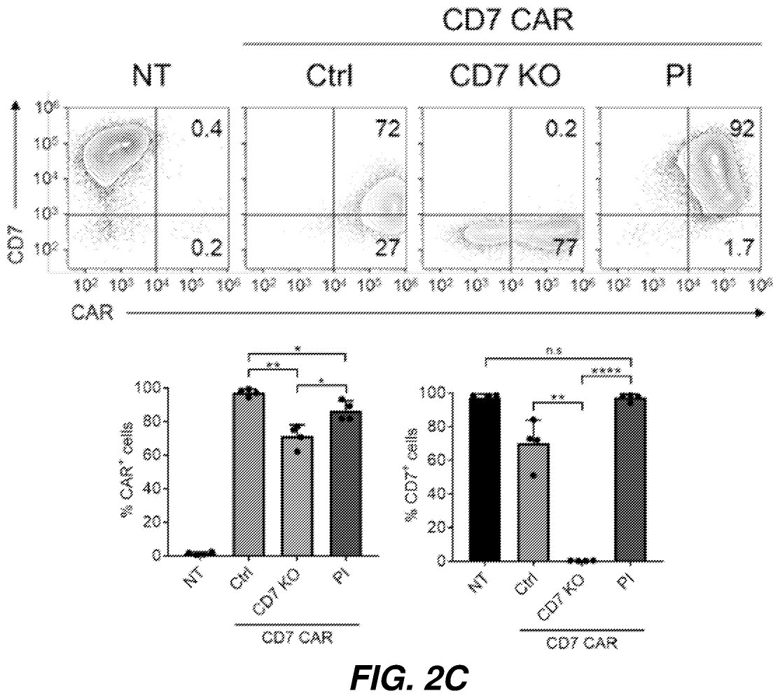

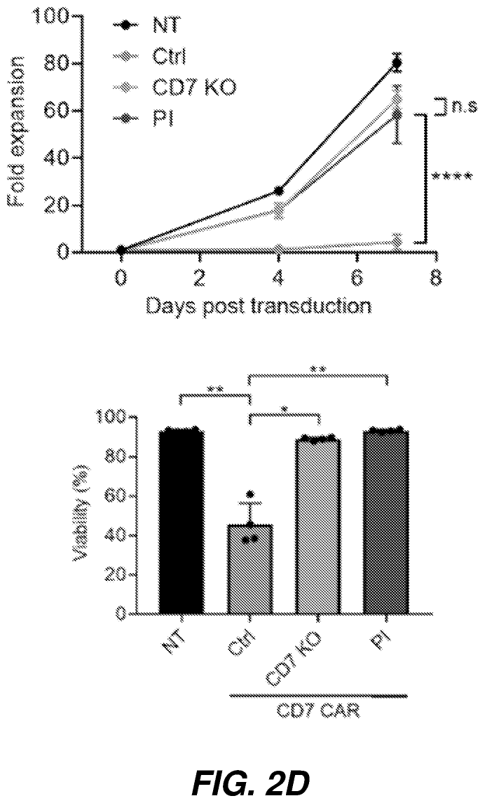

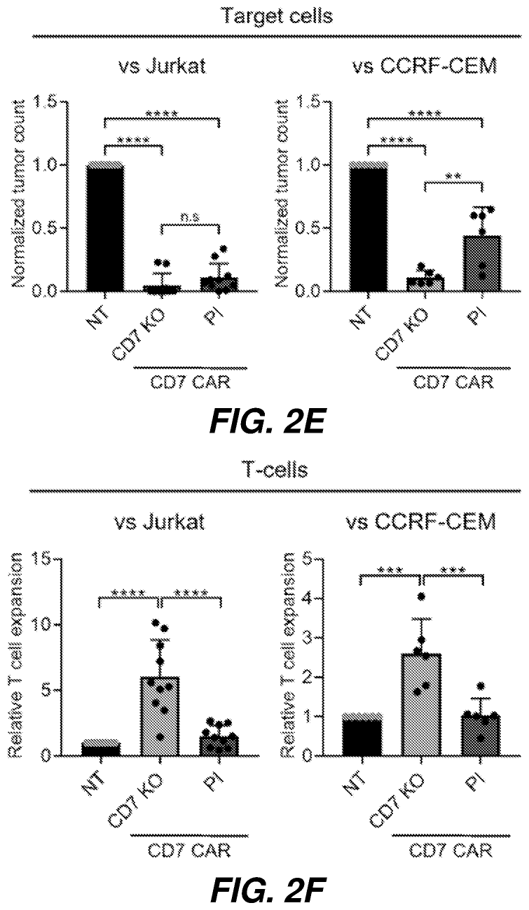

Because most T-cells express high levels of CD7, transduction with a CD7 CAR induces strong fratricide²⁻⁴. In some embodiments, this fratricide can be minimized by pharmacologic inhibition of signaling emanating from the CAR-embedded CD3ξ and CD28 endodomains. Because both molecules activate cytotoxic signaling in T-cells via Lck/Fyn and Itk kinases, dasatinib and ibrutinib were utilized to selectively inhibit these signaling mediators and suppress unwanted CAR-driven cytolysis (FIG. 2A).

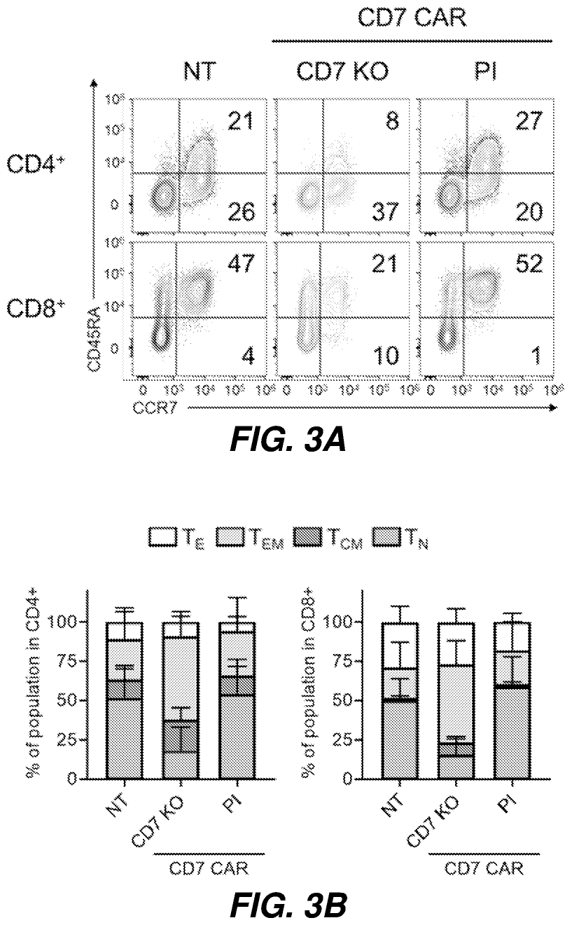

Healthy donor peripheral blood mononuclear cells (PBMCs) were stimulated with anti-CD3/anti-CD28 antibodies in the presence of 200 nM of ibrutinib followed by gammaretroviral transduction with a CD7 CAR vector (FIG. 2B). Dasatinib was added on the day of transduction at the final concentration of 200 nM. Transduced CD7 CAR T-cells were expanded in the presence of both ibrutinib and dasatinib, IL-7, and IL-15. The chemical inhibitors were replenished together with cytokines and fresh medium every 2-3 days. These CD7−unedited CD7 CAR T-cells expanded with the pharmacologic inhibitors (hereafter PI CAR T-cells) retained surface expression of the CAR and had reduced intensity of CD7, possibly due to antigen masking by the CAR (FIG. 2C). Control unedited CD7 CAR T-cells cultured without ibrutinib and dasatinib (FIG. 2B) had high CAR expression with a moderate reduction of surface levels of CD7 (FIG. 2C) and showed abrogated cell expansion and extensive fratricide within a week after CAR transduction (FIG. 2D). In contrast, both CD7−edited CD7 CAR T-cells, where the expression of the CD7 gene was disrupted using CRISPR/Cas9 prior to CAR transduction (FIG. 2B, hereafter CD7 KO CAR T-cells), and PI CAR T-cells retained high viability and produced normal expansion ex vivo (FIG. 2D), indicating that, in some embodiments, pharmacological blockade can prevent unwanted CAR activation and minimize fratricide. Pharmacologic inhibition of CAR signaling also preserved minimally differentiated T-cell populations having a similar phenotype and subset composition to control non-transduced T-cells, whereas these cells were partially depleted in CD7 KO CAR T-cells due to residual CAR signaling (FIGS. 3A, 3B). These data indicate that the expansion of unedited CD7 CAR T-cells in the presence of ibrutinib and dasatinib minimizes deleterious CAR signaling and the resulting fratricide as well as terminal differentiation of T-cells.

Of note, the CD28 costimulatory endodomain also directly recruits the p85 subunit of PI(3) K and activates downstream Akt-mTOR and NF-kB pathways, further contributing to T-cell proliferation and effector differentiation. Neither ibrutinib nor dasatinib is known to directly inhibit the PI (3) K-Akt pathway, and therefore it may still remain active in PI CAR T-cells. If so, this signaling axis did not accelerate T-cell differentiation, as the subset composition of PI CAR T-cells closely resembled that of control donor-matched non-transduced T-cells.

### 2. PI CAR T-Cells Regain Cytotoxicity Upon Removal of Ibrutinib and Dasatinib

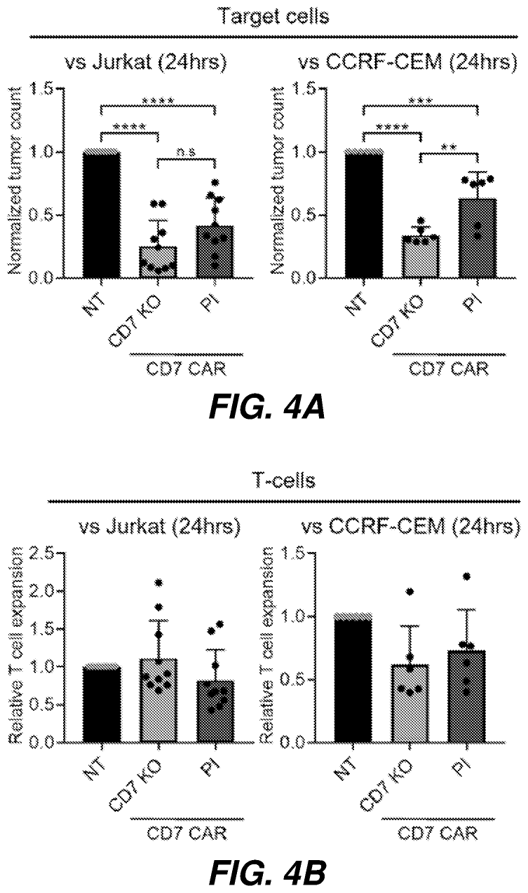

Blockade of CAR signaling protects PI CAR T-cells from fratricide but also inhibits tumor-directed cytotoxicity. To test whether PI CAR T-cells regain their anti-tumor function upon withdrawal of ibrutinib and dasatinib, unedited CD7 CAR T-cells were generated from multiple healthy donors and expanded them ex vivo in the presence of ibrutinib and dasatinib for seven days, following which the T-cells were washed and cryopreserved (FIG. 2B). After thawing, PI CAR T-cells were co-cultured with the CD7⁺ T-ALL cell lines Jurkat or CCRF-CEM for 72h in the absence of ibrutinib, dasatinib, or exogenous cytokines. PI CAR T-cells produced significant cytotoxicity against both cell lines, though CCRF-CEM cell killing was attenuated in some donors compared to CD7 KO CAR T-cells (FIG. 2E). Tumor killing was observed as early as 24h post-thaw, indicating rapid acquisition of cytotoxic effector function upon withdrawal of the pharmacologic inhibitors (FIGS. 4A, 4B). As expected, unblocking CAR signaling also led to resumed fratricide of PI CAR T-cells, reducing their expansion during coculture (FIG. 2F). These studies demonstrate that, in some embodiments, removal of ibrutinib and dasatinib restores CD7-directed cytotoxicity in PI CAR T-cells.

Importantly, the final cell product does not contain any physiologically significant concentrations of dasatinib and ibrutinib as the cells undergo 4 rounds of washing and are reconstituted in a freezing medium devoid of dasatinib and ibrutinib. Residual levels of free (unbound) dasatinib and ibrutinib in the final product were estimated based on the overall dilution during the final washing steps prior to cryopreservation. All cells undergo four washes with approximately 30-fold dilution of the original conditioning medium in each. Collectively, this results in a 30⁴=8.1×10⁵-fold dilution which would reduce the concentration of dasatinib from 500 nM to ˜600 fM and ibrutinib from 200 nM to ˜250 fM. These calculations also overestimate the presence of both compounds in the final product, as they do not account for degradation of dasatinib and ibrutinib in conditioning medium in the days between addition and cryopreservation as well as binding of these inhibitors to target kinases in T-cells, which would further reduce bioavailability of both chemicals. These concentrations are also below the lowest limit of quantification (LLOQ) of dasatinib and ibrutinib in validated LC-MS assays^{39,40}. Finally, the calculated concentrations of dasatinib and ibrutinib in the final product are approximately 100,000-to 1,000,000-fold lower than peak plasma levels (˜30-100 ng/ml or 60-200 nM) in patients receiving dasatinib or ibrutinib in FDA-approved formulations (SPRYCEL® and IMBRUVICA®, respectively)^{41,42}. Considering the above factors as well as the dilution of the administered drug product in circa 4L of peripheral blood and extensive metabolism of dasatinib and ibrutinib in the liver by the CYP3A^{43,44}, residual amounts of both compounds in the final formulation were estimated to be negligible.

### 3. PI CAR T-Cells Produce Robust Anti-Leukemic Activity In Vivo

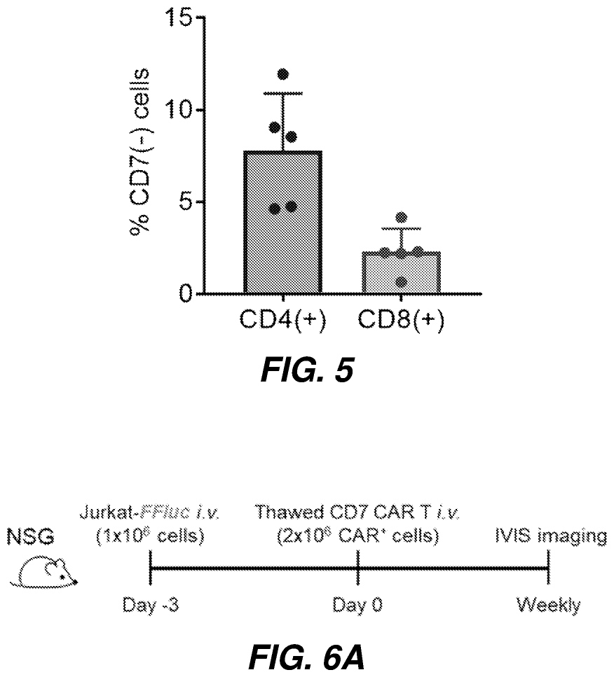

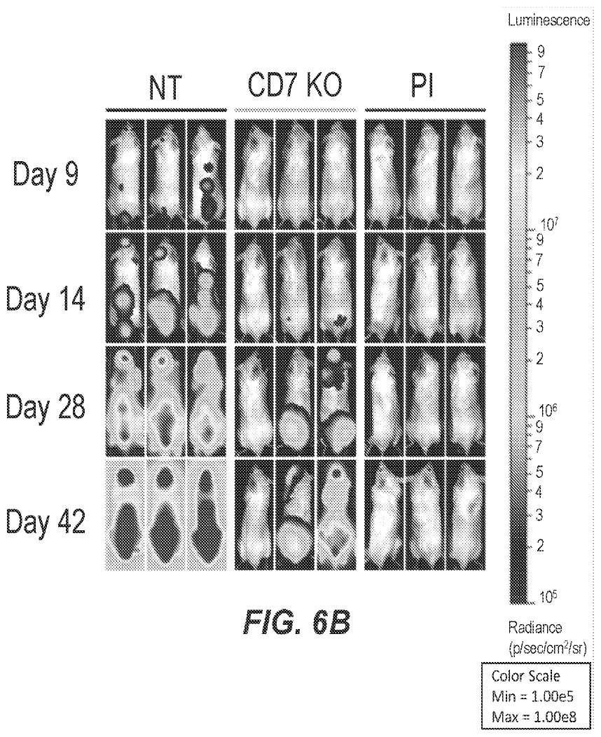

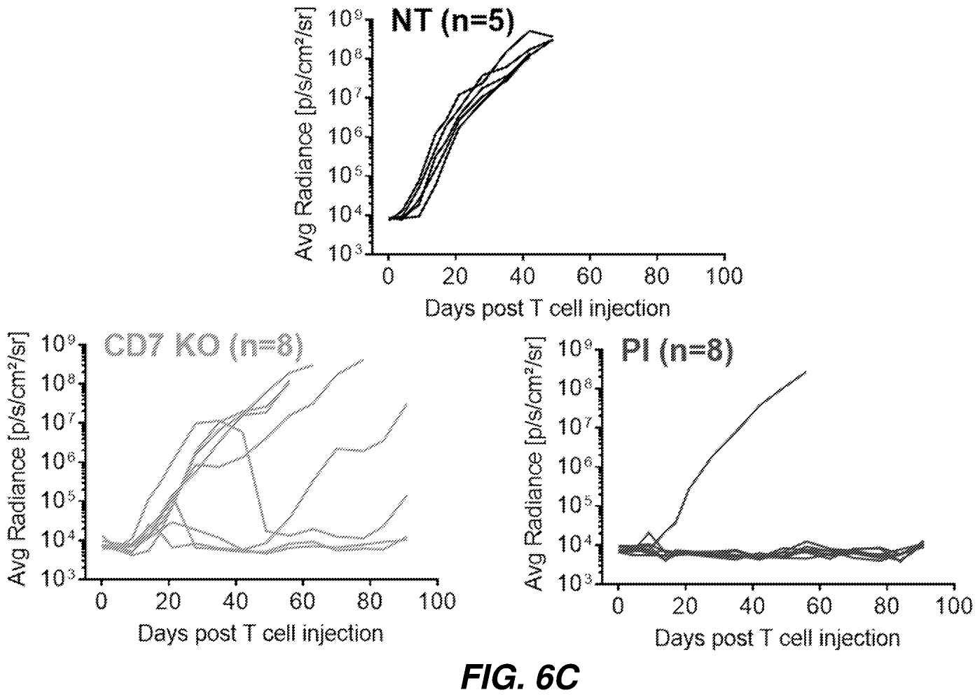

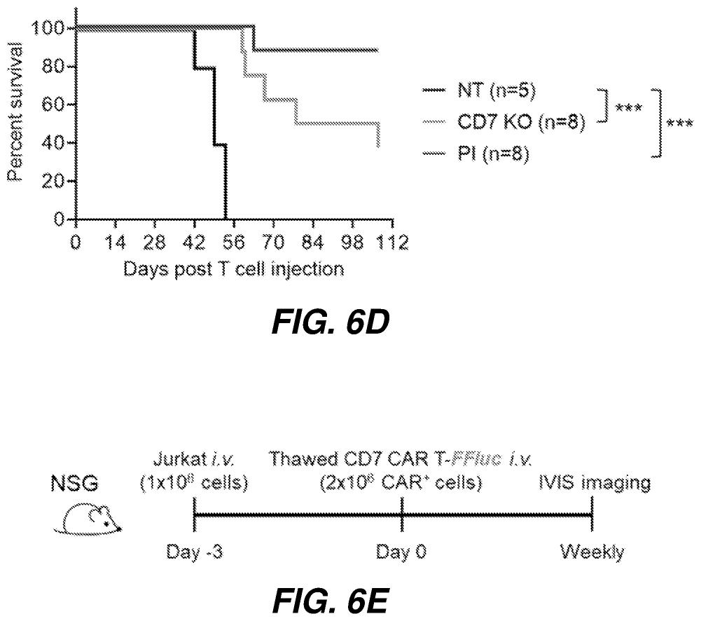

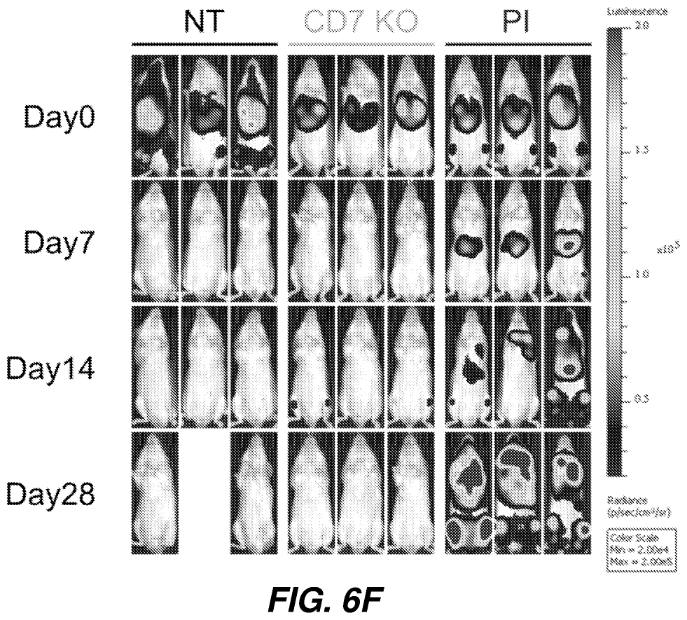

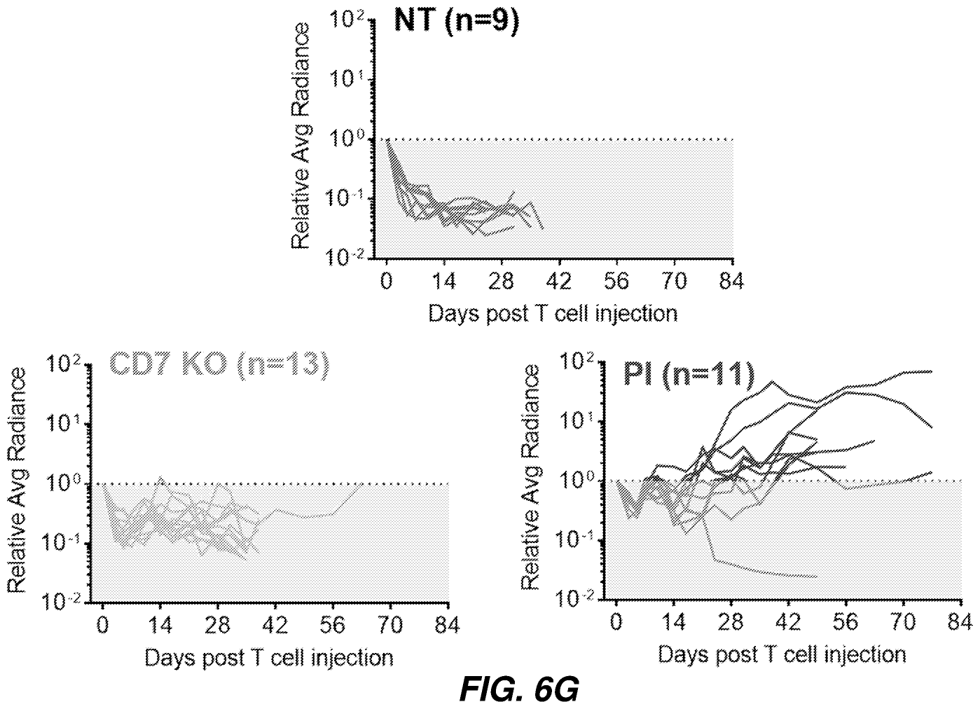

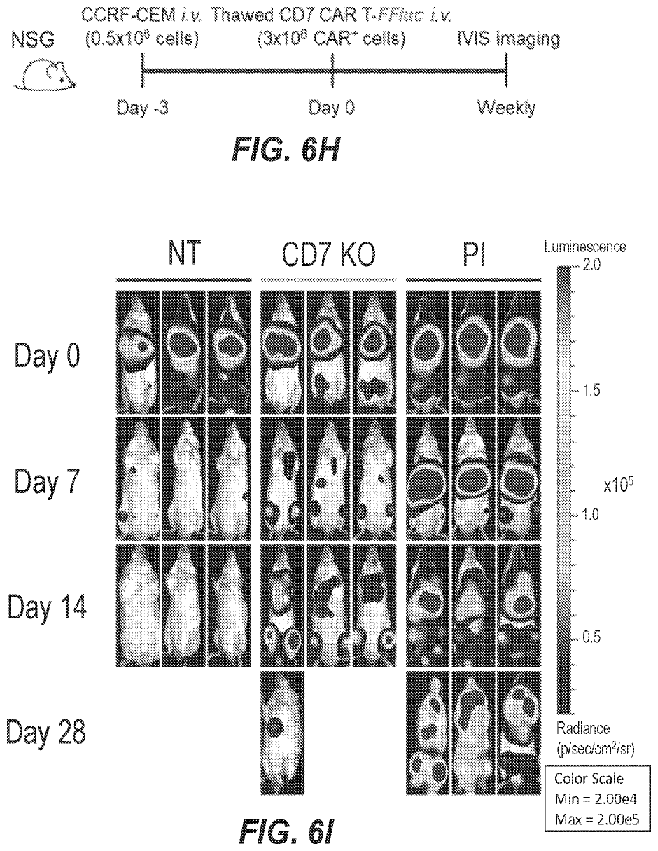

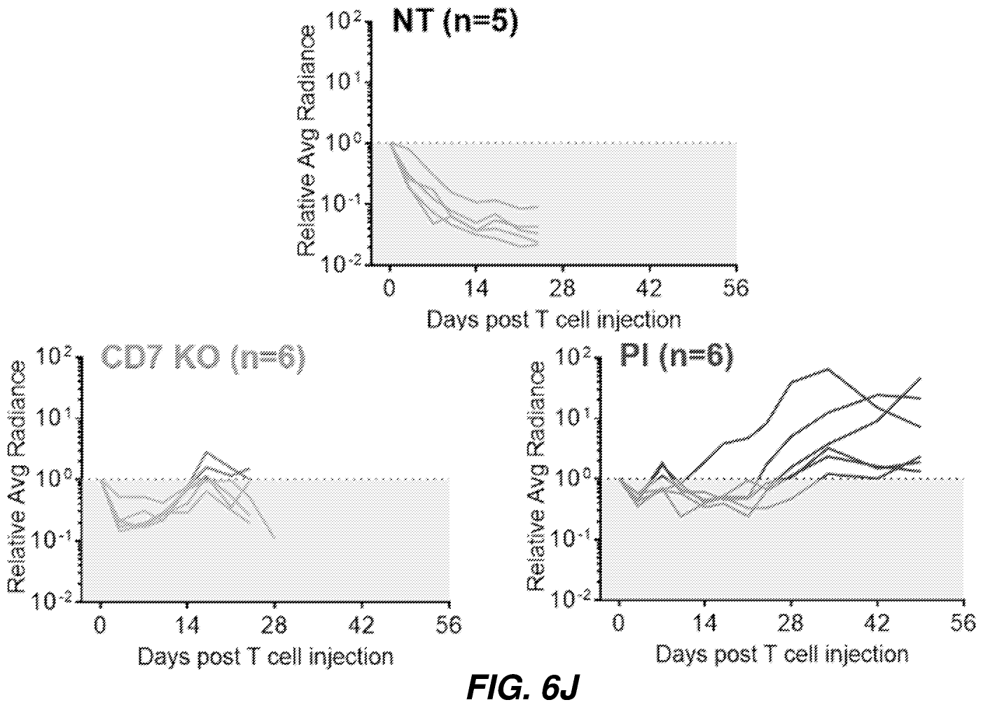

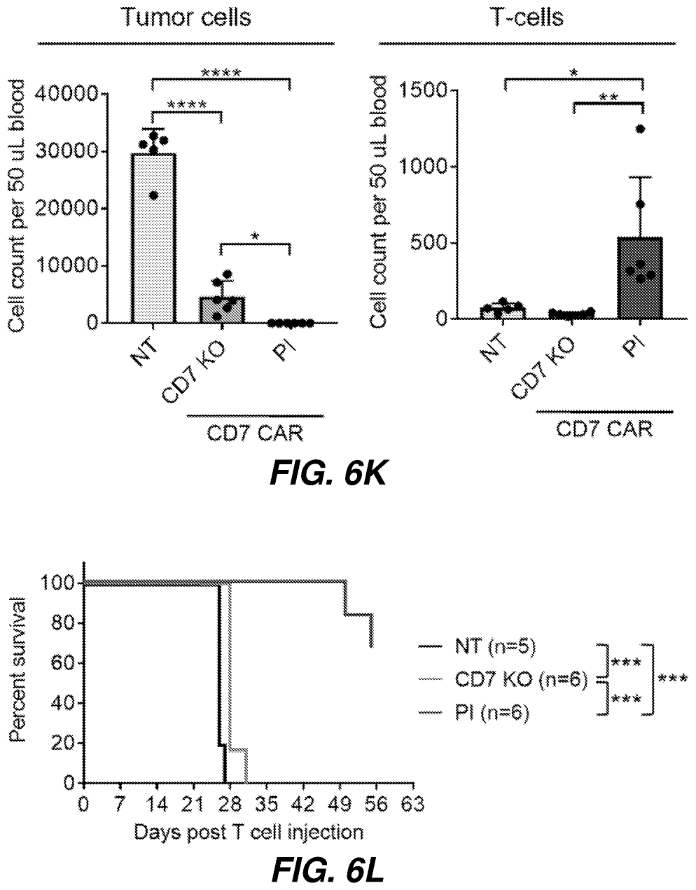

While most T-cells are CD7-positive, a subset naturally lacks CD7 expression. This population is highly variable in frequency among healthy donors, constituting a mean of 7.8% of CD4⁺ and 2.3% of CD8⁺ T-cells (FIG. 5). These cells are expected to resist CD7-directed fratricide and therefore can produce sustained anti-tumor activity. To test the ability of CD7 CAR T-cells to control systemic T-ALL in vivo, CD7⁺ Jurkat T-ALL cells that were modified to express firefly luciferase (FFluc) were engrafted in NSG mice, and freshly thawed CD7 CAR T-cells were injected intravenously three days later (FIG. 6A). While all mice receiving control non-transduced T-cells developed fatal systemic leukemia, PI CAR T-cells mediated potent anti-tumor activity and protected most animals from disease progression, with a single dose of PI CAR T-cells sufficient to prevent tumor growth in seven of eight animals (FIGS. 6B, 6C), thereby significantly extending survival (FIG. 6D). No toxicities were observed in mice treated with CD7 CAR T-cells for the entire duration of the experiment. Thus, in some embodiments, PI CAR T-cells target cancerous T-cells early post-infusion and eventually self-select for a fratricide-resistant, CD7-negative population of CD7 CAR T-cells.

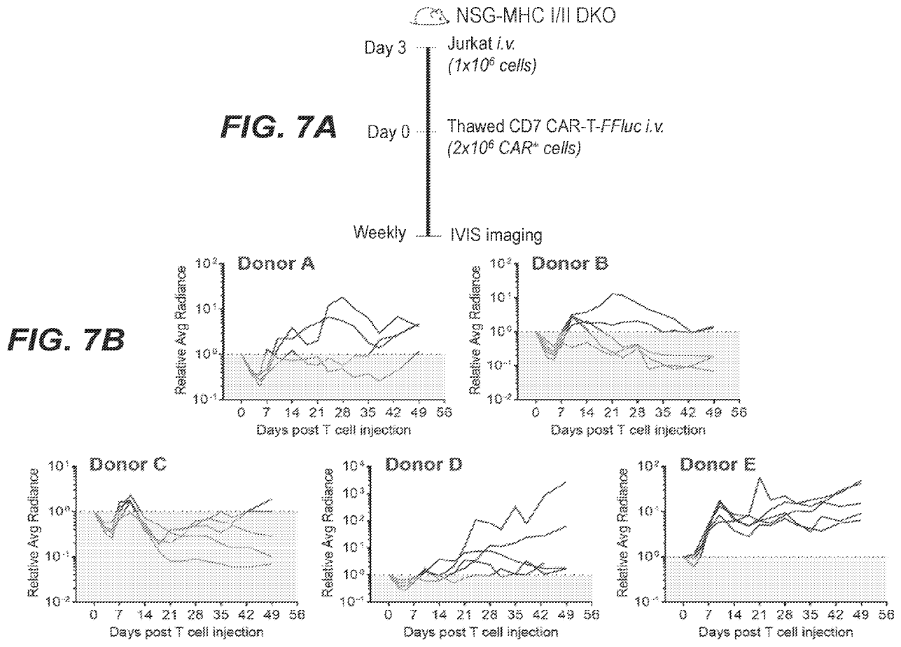

To better characterize the kinetics of expansion and persistence of PI CAR T-cells and how the restored self-targeting capacity of PI CAR T-cells affected antitumor activity in leukemia-bearing mice, FFluc-labeled CD7 CAR T-cells were generated and administered to mice engrafted with Jurkat T-ALL three days prior (FIG. 6E). PI CAR T-cells expanded and persisted in most animals, protecting them from leukemia progression (FIGS. 6F, 6G). CD7 KO CAR T-cells had inferior persistence and anti-tumor activity compared to PI CAR T-cells. Their decreased function in vivo correlated with enhanced terminal differentiation of CD7 KO CAR T-cells (FIG. 3). The long-term persistence and anti-tumor activity of PI CAR T-cells was not specific to a particular T-cell donor or the result of xenogeneic graft-versus-host responses, as similar outcomes with PI CAR T-cells derived from multiple donors in NSG-MHC I/II DKO mice engrafted with Jurkat T-ALL were observed (FIGS. 7A, 7B), albeit with varying magnitude of expansion.

The activity of PI CAR T-cells was also evaluated in a second model of T-ALL in which NSG mice were inoculated with CCRF-CEM T-cell leukemia followed by a single dose of FFluc-labeled CAR T-cells three days later (FIG. 6H). Compared to the Jurkat model, CCRF-CEM produces more aggressive tumors with typical leukemic distribution of malignant cells in peripheral blood and bone marrow⁹. Again, a single injection of PI CAR T-cells led to long-term persistence and anti-tumor activity, eradicating T-ALL blasts in peripheral blood and extending animal survival compared to both non-transduced T-cells and CD7 KO CAR T-cells (FIGS. 6I, 6K). Overall, these results indicate that, in some embodiments, PI CAR T-cells resist fratricide in vivo and produce sustained anti-leukemia activity in mouse xenograft models of human T-ALL.

In most clinical scenarios, infused CAR T-cells are initially surrounded by malignant cells, meaning a CD7-unedited CD7 CAR T-cell will likely encounter a leukemic cell before targeting another CAR T-cell. Therefore, in some embodiments, CD7⁺PI CAR T-cells contribute to short-term anti-leukemic activity before succumbing to fratricide, and in the long-term, CD7⁻ PI CAR T-cells establish more sustained persistence and cytotoxicity.

These result support the potential of adopting CD7-negative T-cells as a platform for engineered cell therapy. CD7 is one of the earliest T-lineage markers expressed in early thymic immigrants, most thymocytes and peripheral T-cells, as well as NK-cells. Functionally, CD7 is a transmembrane protein that provides costimulation and modulates adhesion in T-cells. However, functional importance of CD7 in peripheral T-cells is not clearly defined, and mice lacking CD7 have a largely unperturbed and competent T-cell compartment. In humans, loss of CD7 has been documented in a small subset of circulating T-cells, which are predominantly CD4+ and have the CD45RA⁻ CD45RO⁺ memory phenotype^{6,7,25}. The frequency of CD7-negative circulating T-cells increases with age²⁵. Expansion of CD7⁻ CD4⁺ and CD8⁺T-cells has also been documented in the settings of viral infections (HIV, EBV), rheumatoid arthritis, and other inflammatory conditions²⁵⁻³¹. These and other studies imply that the lack of CD7 is associated with the terminal differentiation of chronically stimulated T-cells but also suggest that T-cells lacking CD7 are more resistant to activation-induced apoptosis³². Data described herein show that CD7⁻ CD7 CAR T-cells persist long-term in immunodeficient mice and suppress leukemia relapse, suggesting that, in some embodiments, cells are capable of producing sustained anti-tumor activity in patients with T-cell malignancies.

### 4. Persisting PI CAR T-Cells Lack CD7 Gene Expression and Transcriptionally Resemble CD7-Edited CAR T-Cells

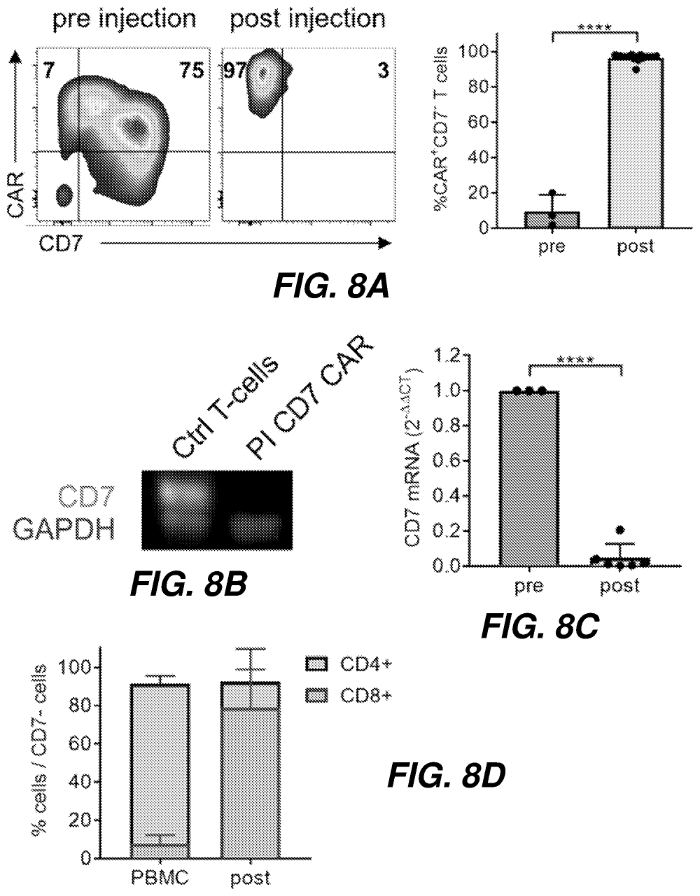

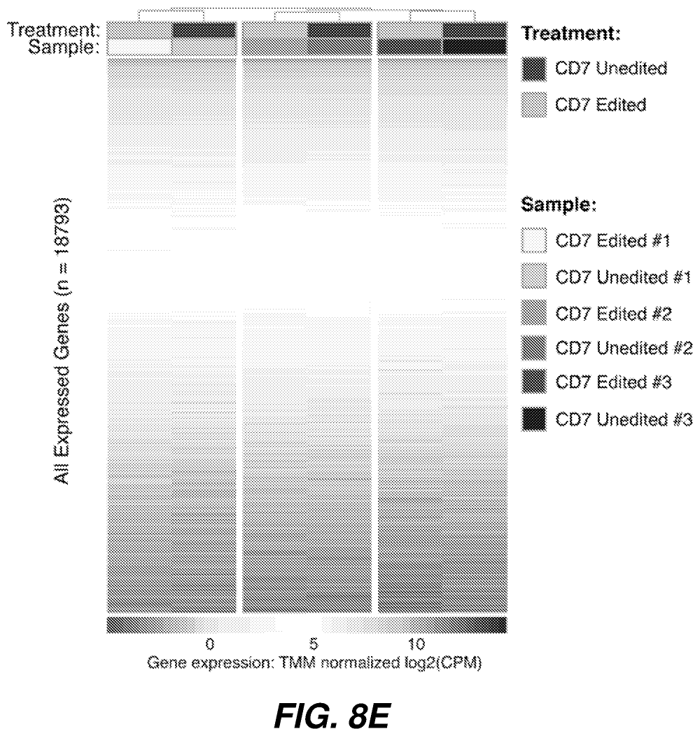

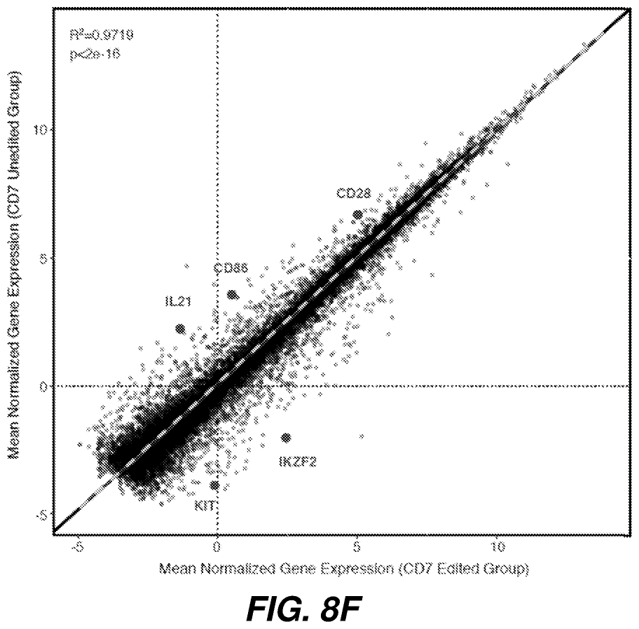

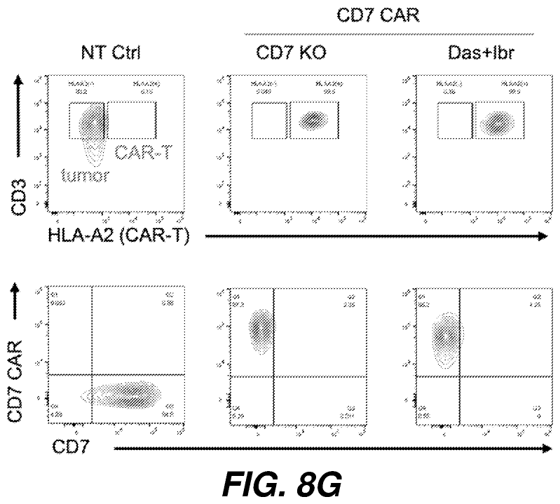

To determine the mechanism of fratricide resistance in PI CAR T-cells in vivo, expression of both the CD7 CAR and the CD7 antigen was measured by flow cytometry on circulating CAR T-cells 27 days post-infusion. In all animals, PI CAR T-cells had uniformly high expression of the CAR whereas CD7 was undetectable (FIG. 8A). Loss of detectable CD7 was not a result of CAR-mediated antigen masking, as PI CAR T-cells lacked both protein and mRNA expression of the CD7 gene, measured by western blot and qPCR, respectively (FIGS. 8B, 8C). These data support the expansion of naturally CD7-negative CAR-transduced T-cells, which are present in peripheral blood of healthy donors. Notably, in most mice, the majority of persisting CD7⁻ CAR T-cells were CD8⁺, in stark contrast to human endogenous PBMC where CD4⁺T-cells dominated the CD7 ⁻ subset, suggesting that CAR signaling favored the expansion of CD8⁺ T-cells in this model (FIG. 8D).

The inventors also analyzed the expression of CD7 CAR and CD7 antigen on infused human T-cells by flow cytometry in peripheral blood of mice on day 32 post T-cell injection. Mice that received non-transduced control T-cells (NT Ctrl) lacked detectable normal T-cells but had a discernible population of circulating leukemic cells, most of which were CD7-positive (FIG. 8G). In contrast, mice that received CD7 CAR T-cells cleared leukemic cells with persistence of CD7 CAR T-cells. Of interest, T-cells in both experimental groups retained CAR expression and had no detectable surface CD7, which correlated with their resistance to fratricide (FIG. 8G). Therefore, in some embodiments, pharmacologic inhibition of CAR signaling during ex vivo expansion is sufficient to generate fratricide-resistant CD7 CAR T-cells without requiring genetic ablation of the target antigen.

Next, the inventors investigated whether the persisting CD7-negative PI CAR T-cells were transcriptionally distinct from control CD7 CAR T-cells in which CD7 gene expression was disrupted by genome editing. CD7-unedited and edited CD7 CAR T-cells that co-expressed FFluc were generated, and both CAR T-cell types were expanded in the presence of both ibrutinib and dasatinib. These CAR T-cells were then injected into NSG mice inoculated with Jurkat T-ALL three days prior. CAR T cells were allowed to expand in tumor-bearing mice for up to 9 weeks. Human T-cells were then purified from mouse spleens, and their transcriptional profile was analyzed using RNA-seq following a brief in vitro expansion. Unsupervised hierarchical clustering analysis revealed the CD7-unedited and CD7-edited CD7 CAR T-cells were transcriptionally very similar (FIG. 8E). The similarity of transcriptomes is also observed in FIG. 8F, where the two transcriptomes of CD7-unedited and CD7-edited CD7 CAR T-cells show a highly significant correlation (R2=0.97; p<2e-16). Out of the near 20,000 genes detected in the cells, only 10² showed a two-fold differential expression (p<0.05) (FIG. 8F). These results indicate that, in some embodiments, the fratricide-resistant PI CAR T-cells lack CD7 expression, persist long-term, and are transcriptionally similar to CD7-edited CD7 CAR T-cells.

5. cGMP Manufacturing of Functional Autologous PI CAR T-Cells for Patients with T-ALL

Pharmacologic inhibition of CAR-driven fratricide offers a straightforward method of cGMP-compliant manufacturing of functional CD7 CAR T-cells without additional genetic engineering. However, as multiple lines of lymphotoxic chemotherapy in patients with treatment-refractory leukemia and lymphoma often change subset composition and the expansion potential of normal circulating T-cells¹⁰, it is important to assess the feasibility of manufacturing functional unedited CD7 CAR T-cells for these patients using a cGMP-compliant method.

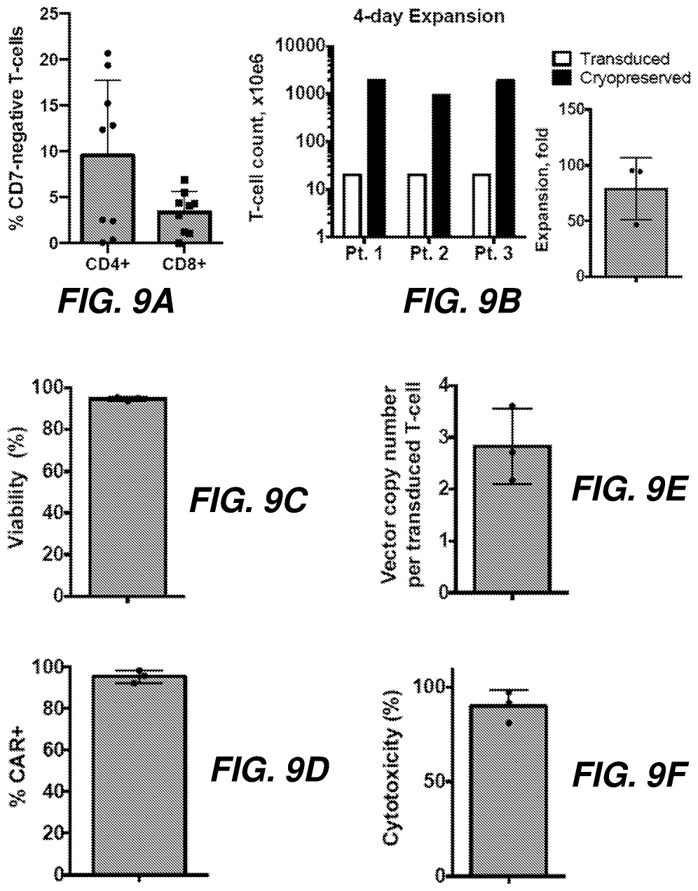

To evaluate the frequency of fratricide-resistant CD7-negative T-cells in the starting cell material, PBMCs of nine patients with CD7⁺ T-cell malignancies were analyzed. CD7-negative T-cells constituted a mean of 9.52% of CD4+ T-cells and a mean of 3.38% of CD8+ T-cells in PBMCs of those patients (FIG. 9A). Based on these data and the preclinical results described above, a Phase I clinical study of autologous unedited CD7 CAR T-cells was initiated in patients with refractory or relapsed T-cell malignancies (CRIMSON-NE, NCT03690011). A cGMP-compliant method of manufacturing PI CAR T-cells was developed and validated by generating CAR T-cell products from adult patients enrolled in the study protocol. PBMCs were obtained from three patients with recalcitrant T-ALL, processed in a cGMP facility, and stimulated with plate-bound CD3− and CD28-specific antibodies in the presence of ibrutinib. Three days later, T-cells were transduced with a clinical-grade CD7 CAR gammaretroviral vector and expanded in the presence of ibrutinib, dasatinib, IL-7 and IL-15.

Robust expansion of PI CAR T-cells was observed in all three patient products with a mean 78.8-fold expansion over four days following transduction (FIG. 9B). At the end of the expansion, CAR T-cells were counted and cryopreserved. Mean viability of cryopreserved CD7 CAR T-cells was 94.7% as measured by flow cytometry (FIG. 9C). CD7 CAR was highly expressed in all three products (mean transduction efficiency 95.3%, FIG. 9D) with mean vector copy number of 2.83 per transduced T-cell (FIG. 9E). Mean cytotoxicity of CD7 CAR T-cells was 90.0% measured in a 24-hour coculture assay with Jurkat T-ALL cells at a 1:2 effector-to-target ratio (FIG. 9F). No residual T-ALL blasts were detected in final products by flow cytometry (data not shown), and all three lines met the release criteria.

### Example 3

### Unedited CD2 Car T-Cells Acquire Resistance to Fratricide and Eradicate Tumor In Vitro

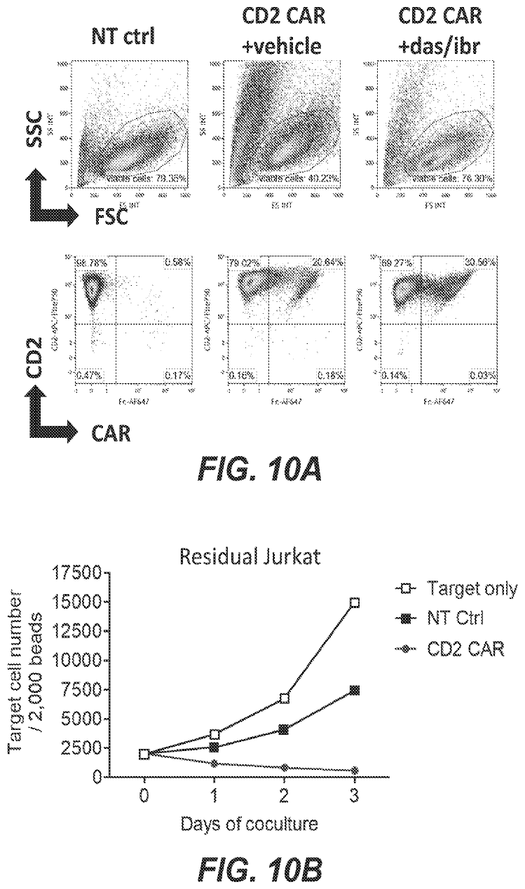

To evaluate whether a similar approach can be extended to antigens beyond CD5 and CD7, the inventors generated CD2 CAR T-cells by gammaretroviral transduction of a CD2 CAR and expanded the CD2 CAR T-cells in the presence of ibrutinib and dasatinib, as described above for CD5 and CD7 CAR T-cells. The resulting CD2 CAR T-cells exhibited normal expansion, retained CD2 expression on the cell surface (FIG. 10B), and produced robust cytotoxicity against a CD2⁺ T-cell line Jurkat (FIG. 10A). Therefore, the methods described herein can be universally applied to generate CAR T-cells targeting fratricidal antigens in addition to CD5 and CD7.

### Example 4

### Exemplary Methods

Donors and cell lines. Peripheral blood mononuclear cells (PBMCs) were obtained from healthy volunteers and patients with T-cell hematologic malignancies. Jurkat, clone E6-1 (acute T cell leukemia cell line) and CCRF-CEM (acute T cell lymphoblastic leukemia cell line) were obtained from the American Type Culture Collection (Rockville, MD). Jurkat and CCRF-CEM cells were maintained in RPMI-1640 medium (GIBCO™ BRL LIFE TECHNOLOGIES™, Inc., Gaithersburg, MD) containing 10% heat-inactivated fetal bovine serum (FBS) (GIBCO™ BRL LIFE TECHNOLOGIES™) with 2 mM L-GLUTAMAX™ (GIBCO™ BRL LIFE TECHNOLOGIES™). Cells were maintained in a humidified atmosphere containing 5% carbon dioxide (CO2) at 37° C. All cell lines have been routinely tested for Mycoplasma.

Generation of retroviral constructs and retrovirus production. A second generation CAR construct targeting CD7 was previously reported by our laboratory^{2,17}. Briefly, the CAR construct is comprised of an scFv domain (clone 3A1e) followed by IgG-derived hinge and C_{H}3 spacer with CD28 transmembrane/costimulatory and CD3ξ signaling domains. The γ-retroviral vectors and the retroviral supernatant were generated as previously described³³.

Generation of CAR-modified T cells and gene-modified cell lines. To obtain activated T cells, 1×10⁶ PBMCs were plated in each well of a non-tissue culture-treated 24-well plate pre-coated with 500 μL of OKT3 (1 mg/mL; Ortho Biotech, Inc., Bridgewater, NJ) and anti-CD28 (1 mg/mL; BD® Biosciences, San Jose, CA) antibodies. Cells were cultured in complete CTL medium containing 45% RPMI-1640 medium, 45% Click's medium (Irvine Scientific), 10% FBS and 2 mM L-GLUTAMAXIM™. IL-7 (10 ng/ml) and IL-15 (10 ng/ml) were added on the following day. To generate CD7-edited CD7 CAR T cells, CD7 gene was genomically disrupted using CRISPR/Cas9 system on day 2 as previously described². CD7 CAR transduction was performed on day 5 where retroviral supernatant was plated in a non-tissue culture-treated 24-well plate pre-coated with recombinant fibronectin fragment (FN CH-296; RETRONECTIN™; TAKARA™ Bio Inc, Otsu, Japan), and centrifuged at 2000 g for 90 min. After removal of the supernatant, OKT3/CD28-activated PBMC were resuspended in complete CTL medium supplemented with IL-7/IL-15 at a final concentration of 0.1×10⁶/mL and 2 mL of cell suspension was added to each virus loaded well, which was subsequently spun at 1000 g for 10 min, and then transferred to a 37° C., 5% CO₂ incubator. In the condition where we generated unedited CD7 CAR T cells in the presence of pharmacologic inhibitors, Ibrutinib (200 nM; Selleckchem, Catalog#S2680) was added on day 0 and a mixture of Dasatinib (200 nM; Selleckchem, Catalog#S1021) and Ibrutinib (200 nM) were added on the day of transduction. For the generation of CD7 CAR and GFP/FFluc co-transduced T cells, GFP/FFluc transduction, CD7 knockout and CD7 CAR transduction was performed on day 2, day 3, and day 6 post initial stimulation, respectively. Transduced cells were transferred to and maintained in tissue culture-treated plates with regular change of CTL medium supplemented with cytokines, Dasatinib (200 nM) and Ibrutinib (200 nM) where needed, and passaged every 2-3 days. To generate tumor cell lines overexpressing GFP/FFLuc, the GFP positive fraction was isolated using a cell sorter (SH800S, Sony Biotechnology, San Jose, CA).

Flow cytometry. Cells were stained with fluorochrome-conjugated antibodies for 20 min at 4° C. All samples were acquired on a Gallios Flow Cytometer (BECKMAN COULTER™ Life Sciences, Indianapolis, IN) or FACSCANTO™ (BD® Bioscience), and data were analyzed using Kaluza 2.1 Flow Analysis Software (Beckman Coulter Life Sciences) or FLOWJO™ (BD® Biosciences). Antibodies used in this study are listed below: ALEXA FLUOR® 647 AFFINIPURE™ Goat Anti-Human IgG, Fcγ fragment specific (Cat#109-605-098, Jackson ImmunoResearch, West Grove, PA), CCR7-FITC (clone 150503, Cat#561271, BD™ Biosciences), CD3-PerCP (clone SK7, Cat#347344, BD® Biosciences), CD45-PE (clone HI30, Cat#555483, BD® Biosciences), CD8-PerCP (clone SK1, Cat#347314, BD® Biosciences), CD3-APC-A750 (clone UCHT1, Cat#A66329, BECKMAN COULTER™ Life Sciences), CD45RA-APC-A750 (clone 2H4LDH11ILDB9 (2H4), Cat#A86050, BECKMAN COULTER™ Life Sciences), CD4-KrO (clone 13B8.2, Cat#A96417, BECKMAN COULTER™ Life Sciences), CD8-PB (clone B9.11, Cat#A82791, BECKMAN COULTER™ Life Sciences), CD7-PC7 (clone CD7-6B7, Cat#343114, BIOLEGEND®, San Diego, CA), CD7-PE (clone CD7-6B7, Cat#343106, BIOLEGEND®), HLA-A2-PB (clone BB7.2, Cat#343312, BIOLEGEND®).

Coculture experiments. In the coculture experiments, freshly thawed 10,000 CAR (+) cells were cocultured with 40,000 GFP (+) target cell lines in 200 μL in one well of 96-well flat-bottom well plates. Cells were harvested and analyzed by flow cytometry on Day 0, Day 1 and Day 3. To quantify cell counts by flow cytometry, 10 μL/sample of COUNTBRIGHT™ Absolute Counting Beads (THERMO FISHER SCIENTIFIC®, INVITROGEN®, Grand Island, NY) was added and 7-AAD (BD® Biosciences) was added to exclude dead cells. Acquisition was halted at 2000 beads. Results were reported as normalized cell counts based on cell counts in control conditions (NT cells+ target cells) at each time point.

In vivo models. Breeder pairs of NOD.Cg-Prkd^{escid I}12rg^{tm1Wjl}/SzJ mice (NSG mice, stock no. 005557) and NOD.Cg-Prkdc^{scid} H2-K1^{tm1Bpe} H2-Ab1^{em1Mvw} H2-D1 ^{tm1Bpe} Il2rg^{tm1Wjl}/SzJ (NSG-MHC I/II DKO mice, stock no. 025216) were purchased from the Jackson Laboratory and bred. Both female and male littermates (aged 8-12 weeks) were used for experiments. To evaluate in vivo anti-tumor effect of CD7 CAR T cells, one million of Jurkat-GFP/FFluc cells were engrafted into each NSG mouse by intravenous injection. Three days later, freshly thawed 2×10⁶ of CD7 CAR T cells were injected intravenously. To track T cell expansion and persistence, Jurkat (1×10⁶ cells/animal) or CCRF-CEM (0.5×10⁶ cells/animal) cells were injected intravenously into either NSG or NSG-MHC I/II DKO mice, and freshly thawed CD7 CAR T cells labeled with GFP/FFluc were injected 3 days later (2×10⁶ CAR+ cells for Jurkat model and 3×10⁶ CAR⁺ cells for CCRF-CEM model). Tumor cell growth or T cell expansion/persistence were evaluated by injecting mice intraperitoneally with 100 μL of D-luciferin (30 mg/mL, PERKINELMER® Inc., Waltham, MA) followed by bioluminescence imaging using an IVIS® Lumina II imaging system (Caliper Life Sciences, Inc., Hopkinton, MA), and analyzed by LIVING IMAGE® software (Caliper Life Sciences, Inc.). To quantify tumor cells and T cells in mouse peripheral blood, 50 μL of blood obtained by tail-vein bleeding was stained with CD3, CD4, CD7, CD8, CD45 and HLA-A2, then treated with RBC Lysis Buffer (BIOLEGEND®) to lyse red blood cells. CD45 (+) CD3 (+) HLA-A2 (+) cells (infused T cells) and CD45 (+) CD3 (+) HLA-A2 (−) cells (tumor cells) were counted by a flow cytometer using COUNTBRIGHT™ Absolute Counting Beads (THERMO FISHER SCIENTIFIC®). To evaluate CAR expression on T cells, mouse peripheral blood was first treated RBC Lysis Buffer, then stained with anti-Fc antibody, washed, and stained with CD3, CD4, CD7, CD8, CD45 and HLA-A2.

Western blot and quantitative PCR. To evaluate CD7 protein and mRNA level in CD7 CAR T cells in vivo, human T cells were extracted from mouse spleen by treating mashed spleen samples with RBC Lysis Buffer. The collected cells were cultured in vitro with IL-7 and IL-15 for 2-4 weeks, and then total protein or total RNA were extracted. At the time of protein/total RNA extraction, more than 95% cells were positive for CD45, CD3 and HLA-A2 (infused T cells) in all samples. For western blot, cell lysates were ran on the MINI-PROTEAN™ Tetra Cell (BIO-RAD™, Hercules, CA) and wet-transferred onto nitrocellulose. Blot was probed with anti-CD7 antibody (Clone: EPR4242, Cat#ab109296, ABCAM™, Waltham, MA) and anti-GAPDH antibody (Clone: 6C5, Cat#sc-32233, SANTA CRUZ BIOTECHNOLOGY®, Dallas, TX) followed by Goat anti-Mouse IRDye 680RD (Cat#925-68070, LI-COR® Biosciences, Lincoln, NE) and Goat anti-Rabbit IRDye 800CW (Cat#925-32211, LI-COR® Biosciences). Blots were developed using the LI-COR® Odyssey® CLx (LI-COR® Biosciences). For quantitative PCR for CD7 mRNA, total RNA was extracted by RNeasy kits (QIAGEN®, Germantown, MD), then complementary DNA was generated by SUPERSCRIPT® III (THERMO FISHER SCIENTIFIC®, INVITROGEN®). Quantitative PCR was performed with ITAQ™ Universal SYBR® Green Supermix (BIO-RAD®) in the CFX85 Real-Time system (BIO-RAD®). Primer sequences used are listed below: ACTB forward; 5′-AGAGCTACGAGCTGCCTGAC-3′, ACTB Reverse; 5′-GGATGCCACAGGACTCCA-3′, CD7 Forward; 5′-CCAGGACAACCTGACTATCACC-3′, CD7 Reverse; 5′-AGCATCTGTGCCATCCTTG-3′.

RNA-sequencing and data analysis. Total RNA samples for quantitative PCR as described above were further treated with RNase-Free DNase (QIAGEN®, Germantown, MD) to remove contaminating genomic DNA. mRNA library preparation and next generation sequencing was performed using ILLUMINA® NOVASEQ™ 6000 (read length: 100 bp paired ends, number of reads per sample: 20 million).

RNA-seq reads were aligned to the human genome (GRCh38, primary assembly) and transcriptome (Gencode version 38 primary assembly gene annotation) using STAR version 2.7.9a. The following non-standard parameters were used for STAR alignment-1-outSAMstrandField intronMotif-outFilterType BySJout-out Filter MultimapNmax alignSJoverhangMin 8-alignSJDBoverhangMin 3-alignEndsType EndToEnd. Only uniquely aligned reads were retained for differential gene expression analysis. Individual gene expression was obtained by counting reads over genes from the same annotation as alignment using featureCounts version 1.5.0-p. Differential gene expression analysis was conducted using DESeq2. Significantly regulated genes were defined as genes with |log 2FC|>1 and FDR <0.05. Unsupervised clustering heatmap was produced using Euclidean clustering.

cGMP manufacturing of unedited CD7 CAR T-cells for patients with T-cell malignancies. Autologous CD7 CAR T-cells were produced in the cGMP facility from patients enrolled on the CRIMSON-NE study using a manufacturing method that closely resembles the research-grade process outlined above. Briefly, freshly thawed PBMC from patients with CD7⁺ T-cell malignancies were plated in T75 flasks coated with anti-CD3/anti-CD28 antibodies in the presence of 200 nM ibrutinib. Three days later, T-cells were collected, counted, and transduced with a clinical-grade gammaretroviral vector encoding CD7 CAR using RETRONECTIN™-coated flasks. Immediately following transduction, dasatinib was added to the final concentration of 500 nM along with recombinant IL-7 (5 ng/ml) and IL-15 (5 ng/mL). Cells were moved to G-Rex culture devices on the next day and allowed to expand in the fresh medium supplemented with ibrutinib, dasatinib, and IL-7/IL-15 cytokines for three additional days. On day 4 post-transduction, T-cells were collected, counted, and cryopreserved according to FDA-approved cGMP SOPs. CAR expression and the presence of malignant T-cells were measured by flow cytometry for each product. Potency of CD7 CAR T-cell products has been assessed by coculturing with CD7⁺ Jurkat T-ALL cells modified to express firefly luciferase and quantifying residual tumor cells by measuring luminescence upon addition of D-luciferin. Average gammaretroviral vector copy number per T-cell has been quantified by qPCR using TAQMAN™ primers specific to CAR sequence.

Statistical analysis. Statistical analysis was performed using GRAPHPAD PRISM® 7 software (GRAPHPAD™ Software, Inc., La Jolla, CA). Statistical tests used in each experiment are described in figure legends.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
1. Scherer, L. D., Brenner, M. K., and Mamonkin, M. (2019). Chimeric Antigen Receptors for T-Cell Malignancies. Front Oncol 9, 126.
2. Gomes-Silva, D., Srinivasan, M., Sharma, S., Lee, C. M., Wagner, D. L., Davis, T. H., Rouce, R. H., Bao, G., Brenner, M. K., and Mamonkin, M. (2017). CD7-edited T cells expressing a CD7-specific CAR for the therapy of T-cell malignancies. Blood 130, 285-296.
3. Png, Y. T., Vinanica, N., Kamiya, T., Shimasaki, N., Coustan-Smith, E., and Campana, D. (2017). Blockade of CD7 expression in T cells for effective chimeric antigen receptor targeting of T-cell malignancies. Blood Adv 1, 2348-2360.
4. Cooper, M. L., Choi, J., Staser, K., Ritchey, J. K., Devenport, J. M., Eckardt, K., Rettig, M. P., Wang, B., Eissenberg, L. G., Ghobadi, A., et al. (2018). An “off-the-shelf” fratricide-resistant CAR-T for the treatment of T cell hematologic malignancies. Leukemia.
5. Pan, J., Tan, Y., Wang, G., Deng, B., Ling, Z., Song, W., Seery, S., Zhang, Y., Peng, S., Xu, J., et al. (2021). Donor-Derived CD7 Chimeric Antigen Receptor T Cells for T-Cell Acute Lymphoblastic Leukemia: First-in-Human, Phase I Trial. JCO 39, 3340-3351.
6. Reinhold, U., Abken, H., Kukel, S., Moll, M., Müller, R., Oltermann, I., and Kreysel, H. W. (1993). CD7-T cells represent a subset of normal human blood lymphocytes. J Immunol 150, 2081-2089.
7. Reinhold, U., Liu, L., Sesterhenn, J., and Abken, H. (1996). CD7-negative T cells represent a separate differentiation pathway in a subset of post-thymic helper T cells. Immunology 89, 391-396.
8. Boomer, J. S., and Green, J. M. (2010). An Enigmatic Tail of CD28 Signaling. Cold Spring Harb Perspect Biol 2, a002436.
9. Mamonkin, M., Rouce, R. H., Tashiro, H., and Brenner, M. K. (2015). A T-cell-directed chimeric antigen receptor for the selective treatment of T-cell malignancies. Blood 126, 983-992.
10. Das, R. K., Vernau, L., Grupp, S. A., and Barrett, D. M. (2019). Naïve T-cell Deficits at Diagnosis and after Chemotherapy Impair Cell Therapy Potential in Pediatric Cancers. Cancer Discov 9, 492-499.
11. Rasaiyaah, J., Georgiadis, C., Preece, R., Mock, U., and Qasim, W. (2018).

TCRaß/CD3 disruption enables CD3-specific antileukemic T cell immunotherapy. JCI Insight 3.
12. Maciocia, P. M., Wawrzyniecka, P. A., Philip, B., Ricciardelli, I., Akarca, A. U., Onuoha, S. C., Legut, M., Cole, D. K., Sewell, A. K., Gritti, G., et al. (2017). Targeting the T cell receptor B-chain constant region for immunotherapy of T cell malignancies. Nature Medicine 23, 1416-1423.
13. Gao, Z., Tong, C., Wang, Y., Chen, D., Wu, Z., and Han, W. (2019).

Blocking CD38-driven fratricide among T cells enables effective antitumor activity by CD38-specific chimeric antigen receptor T cells. J Genet Genomics 46, 367-377.
14. Breman, E., Demoulin, B., Agaugué, S., Mauën, S., Michaux, A., Springuel, L., Houssa, J., Huberty, F., Jacques-Hespel, C., Marchand, C., et al. (2018). Overcoming Target Driven Fratricide for T Cell Therapy. Front Immunol 9, 2940.
15. Long, A. H., Haso, W. M., Shern, J. F., Wanhainen, K. M., Murgai, M., Ingaramo, M., Smith, J. P., Walker, A. J., Kohler, M. E., Venkateshwara, V. R., et al. (2015). 4-1B B costimulation ameliorates T cell exhaustion induced by tonic signaling of chimeric antigen receptors. Nat Med 21, 581-590.
16. Mamonkin, M., Mukherjee, M., Srinivasan, M., Sharma, S., Gomes-Silva, D., Mo, F., Krenciute, G., Orange, J. S., and Brenner, M. K. (2018). Reversible Transgene Expression Reduces Fratricide and Permits 4-1B B Costimulation of CAR T Cells Directed to T-cell Malignancies. Cancer Immunol Res 6, 47-58.
17. Gomes-Silva, D., Atilla, E., Atilla, P. A., Mo, F., Tashiro, H., Srinivasan, M., Lulla, P., Rouce, R. H., Cabral, J. M. S., Ramos, C. A., et al. (2019). CD7 CAR T Cells for the Therapy of Acute Myeloid Leukemia. Molecular Therapy 27, 272-280.
18. Kamiya, T., Wong, D., Png, Y. T., and Campana, D. (2018). A novel method to generate T-cell receptor-deficient chimeric antigen receptor T cells. Blood Advances 2, 517-528.
19. Mo, F., Watanabe, N., McKenna, M. K., Hicks, M. J., Srinivasan, M., Gomes-Silva, D., Atilla, E., Smith, T., Ataca Atilla, P., Ma, R., et al. (2021). Engineered off-the-shelf therapeutic T cells resist host immune rejection. Nat Biotechnol 39, 56-63.
20. Pinz, K., Liu, H., Golightly, M., Jares, A., Lan, F., Zieve, G. W., Hagag, N., Schuster, M., Firor, A. E., Jiang, X., et al. (2016). Preclinical targeting of human T-cell malignancies using CD4-specific chimeric antigen receptor (CAR)-engineered T cells. Leukemia 30, 701-707.
21. Weber, E. W., Lynn, R. C., Sotillo, E., Lattin, J., Xu, P., and Mackall, C. L. (2019). Pharmacologic control of CAR-T cell function using dasatinib. Blood Advances 3, 711-717.
22. Mestermann, K., Giavridis, T., Weber, J., Rydzek, J., Frenz, S., Nerreter, T., Mades, A., Sadelain, M., Einsele, H., and Hudecek, M. (2019). The tyrosine kinase inhibitor dasatinib acts as a pharmacologic on/off switch for CAR T cells. Science Translational Medicine 11.
23. Weber, E. W., Parker, K. R., Sotillo, E., Lynn, R. C., Anbunathan, H., Lattin, J., Good, Z., Belk, J. A., Daniel, B., Klysz, D., et al. (2021). Transient rest restores functionality in exhausted CAR-T cells through epigenetic remodeling. Science.
24. Fan, F., Yoo, H. J., Stock, S., Wang, L., Liu, Y., Schubert, M.-L., Wang, S., Neuber, B., Hückelhoven-Krauss, A., Gern, U., et al. (2021). Ibrutinib for improved chimeric antigen receptor T-cell production for chronic lymphocytic leukemia patients. International Journal of Cancer 148, 419-428.
25. Reinhold, U., and Abken, H. (1997). CD4+ CD7-T cells: a separate subpopulation of memory T cells? J Clin Immunol 17, 265-271.
26. Smith, K. J., Skelton, H. G., Chu, W. S., Yeager, J., Angritt, P., and Wagner, K. F. (1995). Decreased CD7 expression in cutaneous infiltrates of HIV-1+ patients. Military Medical Consortium for the Advancement of Retroviral Research (MMCARR). Am J Dermatopathol 17, 564-569.
27. Weisberger, J., Cornfield, D., Gorczyca, W., and Liu, Z. (2003). Down-regulation of pan-T-cell antigens, particularly CD7, in acute infectious mononucleosis. Am J Clin Pathol 120, 49-55.
28. Lazarovits, A. I., White, M. J., and Karsh, J. (1992). CD7-T cells in rheumatoid arthritis. Arthritis Rheum 35, 615-624.
29. Klameth, A., Neubauer, A., Keller, C., Aepinus, C., Kaiser, U., Hoffmann, J., and Brendel, C. (2020). Aberrant CD3-Positive, CD8-Low, CD7-Negative Lymphocytes May Appear During Viral Infections and Mimic Peripheral T-Cell Lymphoma. Diagnostics (Basel) 10, E204.
30. Schmidt, D., Goronzy, J. J., and Weyand, C. M. (1996). CD4+ CD7− CD28− T cells are expanded in rheumatoid arthritis and are characterized by autoreactivity. J Clin Invest 97, 2027-2037.
31. Moll, M., Reinhold, U., Kukel, S., Abken, H., Müller, R., Oltermann, I., and Kreysel, H. W. (1994). CD7-negative helper T cells accumulate in inflammatory skin lesions. J Invest Dermatol 102, 328-332.
32. Wallace, D. L., Matear, P. M., Davies, D. C., Hicks, R., Lebosse, C., Eyeson, J., Beverley, P. C., and Vyakarnam, A. (2000). CD7 expression distinguishes subsets of CD4 (+) T cells with distinct functional properties and ability to support replication of HIV-1. Eur J Immunol 30, 577-585.
33. Mo, F., and Mamonkin, M. (2020). Generation of Chimeric Antigen Receptor T Cells Using Gammaretroviral Vectors. Methods Mol Biol 2086, 119-130.
34. Mair-Jenkins J, Saavedra-Campos M, Baillie J K, et al. The effectiveness of convalescent plasma and hyperimmune immunoglobulin for the treatment of severe acute respiratory infections of viral etiology: a systematic review and exploratory meta-analysis. J Infect Dis 2015; 211 (1): 80-90.
35. Goldman J D, Lye D C, Hui D S, et al. Remdesivir for 5 or 10 days in patients with severe Covid-19. New England Journal of Medicine 2020.
36. Spinner C D, Gottlieb R L, Criner G J, et al. Effect of Remdesivir vs Standard Care on Clinical Status at 11 Days in Patients With Moderate COVID-19: A Randomized Clinical Trial. Jama 2020; 324 (11): 1048-57.
37. Wang Y, Zhang D, Du G, et al. Remdesivir in adults with severe COVID-19: a randomised, double-blind, placebo-controlled, multicentre trial. The Lancet 2020.
38. Group R C. Dexamethasone in hospitalized patients with Covid-19-preliminary report. New England Journal of Medicine 2020.
39. Baum A, Fulton B O, Wloga E, et al. Antibody cocktail to SARS-COV-2 spike protein prevents rapid mutational escape seen with individual antibodies. Science 2020; 369 (6506): 1014-8.
40. Cohen J. ‘Provocative results’ boost hopes of antibody treatment for COVID-19. Science 2020.
41. Duan K, Liu B, Li C, et al. Effectiveness of convalescent plasma therapy in severe COVID-19 patients. Proceedings of the National Academy of Sciences 2020; 117 (17): 9490-6.
42. Shen C, Wang Z, Zhao F, et al. Treatment of 5 critically ill patients with COVID-19 with convalescent plasma. Jama 2020; 323 (16): 1582-9.
43. Li L, Zhang W, Hu Y, et al. Effect of Convalescent Plasma Therapy on Time to Clinical Improvement in Patients With Severe and Life-threatening COVID-19: A Randomized Clinical Trial. Jama 2020.
44. Ibrahim J G C M, Sinha, D. Bayesian survival analysis. Springer-Verlag, Inc, New York Ibrahim J G, Chen M H, Sinha D (2004) Bayesian methods for joint modeling of longitudinal and survival data with applications to cancer vaccine studies. Stat Sin 2001; 14:863883.
45. Spiegelhalter D J, Best N G, Carlin B P, Van Der Linde A. Bayesian measures of model complexity and fit. Journal of the Royal Statistical Society: Series β (Statistical Methodology) 2002; 64 (4): 583-639.
46. Horby P, Lim W S, Emberson J R, et al. Dexamethasone in Hospitalized Patients with Covid-19-Preliminary Report. The New England Journal of Medicine 2020.
47. Cavalli G, De Luca G, Campochiaro C, et al. Interleukin-1 blockade with high-dose anakinra in patients with COVID-19, acute respiratory distress syndrome, and hyperinflammation: a retrospective cohort study. The Lancet Rheumatology 2020.
48. Xu X, Han M, Li T, et al. Effective Treatment of Severe COVID-19 Patients with Tocilizumab. ChinaXiv: 20200300026; 2020.
49. Guaraldi G, Meschiari M, Cozzi-Lepri A, et al. Tocilizumab in patients with severe COVID-19: a retrospective cohort study. The Lancet Rheumatology 2020; 2 (8): e474-e84.
50. Joyner M J, Bruno K A, Klassen S A, et al. Safety update: COVID-19 convalescent plasma in 20,000 hospitalized patients. Mayo Clinic Proceedings; 2020: Elsevier; 2020.
51. Salazar E, Christensen P A, Graviss E A, et al. Treatment of COVID-19 Patients with Convalescent Plasma Reveals a Signal of Significantly Decreased Mortality. The American Journal of Pathology 2020.
52. Joyner M J, Senefeld J W, Klassen S A, et al. Effect of Convalescent Plasma on Mortality among Hospitalized Patients with COVID-19: Initial Three-Month Experience. medRxiv 2020.
53. Woo PCY, Lau SKP, Chu C-m, et al. Characterization and Complete Genome Sequence of a Novel Coronavirus, Coronavirus HKU1, from Patients with Pneumonia. Journal of Virology 2005; 79 (2): 884-95.
54. Mounir S, Talbot P J. Molecular characterization of the S protein gene of human coronavirus OC43. Journal of General Virology 1993; 74 (9): 1981-7.
55. Clausen™, Sandoval D R, Spliid C B, et al. SARS-COV-2 Infection Depends on Cellular Heparan Sulfate and ACE2. Cell 2020.
56. Wec A Z, Wrapp D, Herbert A S, et al. Broad neutralization of SARS-related viruses by human monoclonal antibodies. Science 2020; 369 (6504): 731-6.
57. Fantini J, Di Scala C, Chahinian H, Yahi N. Structural and molecular modelling studies reveal a new mechanism of action of chloroquine and hydroxychloroquine against SARS-COV-2 infection. International Journal of Antimicrobial Agents 2020; 55 (5): 105960.
58. Tortorici M A, Walls A C, Lang Y, et al. Structural basis for human coronavirus attachment to sialic acid receptors. Nature Structural & Molecular Biology 2019; 26 (6): 481-9.
59. Hulswit RJG, Lang Y, Bakkers MJG, et al. Human coronaviruses OC43 and

HKUI bind to 9-O-acetylated sialic acids via a conserved receptor-binding site in spike protein domain A. Proceedings of the National Academy of Sciences 2019; 116 (7): 2681-90.
60. Morniroli D, Giannì M L, Consales A, Pietrasanta C, Mosca F. Human Sialome and Coronavirus Disease-2019 (COVID-19) Pandemic: An Understated Correlation? Frontiers in Immunology 2020; 11 (1480).
61. Sagar M, Reifler K, Rossi M, et al. Recent endemic coronavirus infection is associated with less severe COVID-19. J Clin Invest 2020.
62. Le Bert N, Tan A T, Kunasegaran K, et al. SARS-COV-2-specific T-cell immunity in cases of COVID-19 and SARS, and uninfected controls. Nature 2020; 584 (7821): 457-62.
63. Mateus J, Grifoni A, Tarke A, et al. Selective and cross-reactive SARS-CoV-2 T-cell epitopes in unexposed humans. Science 2020; 370 (6512): 89-94.

What is claimed is:

1. A composition comprising an effective amount of a population of genetically engineered immune cells comprising one or more chimeric antigen receptors (CARs) and/or T-cell receptors (TCRs),
wherein the population of genetically engineered immune cells or a subset thereof express one or more target antigens to which the one or more CARs and/or TCRs specifically bind,
wherein signaling by the one or more CARs and/or TCRs upon binding of the one or more target antigens expressed by the population of genetically engineered immune cells or a subset thereof by the one or more CARs and/or TCRs is reduced upon culture of a population of immune cells manipulated to express the one or more CARs and/or TCRs and/or the population of genetically engineered immune cells in the presence of one or more tyrosine kinase inhibitors (TKIs), and
wherein a reduction in signaling by the one or more CARs and/or TCRs reduces immune cell activation, differentiation, and/or fratricide by the population of genetically engineered immune cells or a subset thereof compared to genetically engineered immune cells cultured in the absence of the one or more TKIs.

2. The composition of claim 1, wherein the immune cells comprise T-cells, Natural Killer (NK) cells, myeloid, B-cells, or a mixture thereof.

3. The composition of claim 1 or claim 2, wherein the immune cells comprise T-cells.

4. The composition of claim 1 or claim 2, wherein the immune cells comprise NK cells.

5. The composition of claim 1 or claim 2, wherein the immune cells comprise myeloid cells.

6. The composition of claim 1 or claim 2, wherein the immune cells comprise B-cells.

7. The composition of any of claims 1-6, wherein the one or more target antigens comprise one or more endogenous gene products expressed by the immune cells.

8. The composition of any of claims 1 to 7, wherein the one or more target antigens comprise CD2, CD5, CD7, CD4, CD8, CD3, CS1, CD38, CD99, CD30, 4-1BB, OX40, ICOS, CD26, CD6, TIGIT, PD-1, 2B4, LAG-3, MHC-I, MHC-II, peptide-MHC I, peptide-MHC II, Tim3, CTLA-4, CD112R, CD226, CD96, CD80, CD86, CD112, CD155, KIR2, KIR3, LILRB, CD28, CD40L, CD40, BTLA, GITR, VISTA, NKG2D ligands, or CD70.

9. The composition of any of claims 1-4, wherein the one or more target antigens comprise one or more antigens acquired via trogocytosis and expressed by the immune cells.

10. The composition of any of claims 1-9, wherein the one or more CARs and/or TCRs comprise one or more antibodies or fragments thereof with specificity against the one or more target antigens.

11. The composition of claim 10, wherein the antibodies or fragments thereof are scFv monoclonal antibodies, nanobodies/VHH-only sequences, fibronectin-derived binding domains, DARPINs, or natural ligands.

12. The composition of any of claims 1-11, wherein the one or more CARs comprise a hinge or spacer comprising a sequence derived from IgG, CD3, CD4, CD5, CD8, CD9, CD16, CD22, CD28, CD33, CD37, CD45, CD64, CD80, CD86, CD134, CD137, CD154, 4-1BB, OX40, a T-cell receptor α or β chain, ICOS, or a combination thereof.

13. The composition of any of claims 1-12, wherein the one or more CARs comprise a hinge or spacer comprising an IgG-derived sequence.

14. The composition of any of claims 1-13, wherein the one or more CARs comprise a hinge comprising an IgG4-derived sequence.

15. The composition of any of claims 1-14, wherein the one or more CARs comprise a spacer comprising an IgG1-derived sequence.

16. The composition of any of claims 1-15, wherein the one or more CARs comprise a C_{H}3 IgG1 spacer.

17. The composition of any of claims 1-16, wherein the one or more CARs comprise one or more signaling domains from CD2, CD3ξ, CD3δ, CD3ε, CD3γ, Fc receptors, CD79a, CD79b, CLEC-2, CD7, LFA-1 (CD11a/CD18), CD27, CD28, CD30, CD40, 4-1BB (CD137), CD278, 2B4, DNAM-1, OX40, NKG2C, NKG2D, DAP10, DAP12, B7-1/CD80, CD28, 4-1BBL, B7-2/CD86, CTLA-4, B7-H1/PD-L1, ICOS, B7-H2, PD-1, B7-H3, PD-L2, B7-H4, PDCD6, HVEM, LIGHT, ICAM-1, BTLA, GITR, or a combination thereof.

18. The composition of any of claims 1-17, wherein the one or more CARs comprise one or more signaling domains from CD3ξ, CD28, 4-1BB, or a combination thereof.

19. The composition of any of claims 1-18, wherein the one or more CARs and/or TCRs are encoded by one or more isolated nucleic acid sequences.

20. The composition of claim 19, wherein the one or more isolated nucleic acid sequences are comprised in one or more expression vectors.

21. The composition of claim 20, wherein the one or more expression vectors are a lentiviral vector, a gamma-retroviral vector, adenoviral vector, an adeno-associated viral vector, or a combination thereof.

22. The composition of any of claims 1-21, wherein the one or more TKIs comprise one or more Src kinase inhibitors.

23. The composition of any of claims 1-22, wherein the one or more TKIs comprise dasatinib, ibrutinib, pp2, pazopanib, gefitinib, or a combination thereof.

24. The composition of any of claims 1-23, wherein at least one of the one or more TKIs comprises dasatinib.

25. The composition of any of claims 1-23, wherein at least one of the one or more TKIs comprises ibrutinib.

26. The composition of any of claims 1-25, wherein the one or more TKIs comprise dasatinib and ibrutinib.

27. The composition of any of claims 1-26, wherein one or more endogenous genes in the population of genetically engineered immune cells or a subset thereof are not inhibited.

28. The composition of any of claims 1-27, further comprising a pharmaceutically acceptable carrier.

29. A method of generating a population of genetically engineered immune cells, the method comprising manipulating a population of immune cells in culture with one or more TKIs to express one or more CARs and/or TCRs to produce the genetically engineered immune cells, wherein the produced population of genetically engineered immune cells or a subset thereof have reduced fratricidal activity in culture compared to genetically engineered immune cells cultured in the absence of the one or more TKIs.

30. The method of claim 29, wherein the immune cells comprise T-cells, Natural Killer (NK) cells, myeloid cells, B-cells, or a mixture thereof.

31. The method of claim 29 or claim 30, wherein the immune cells comprise T-cells.

32. The method of claim 29 or claim 30, wherein the immune cells comprise NK cells.

33. The composition of claim 29 or claim 30, wherein the immune cells comprise myeloid cells.

34. The composition of claim 29 or claim 30, wherein the immune cells comprise B-cells.

35. The method of any of claims 29-34, wherein the population of genetically engineered immune cells or a subset thereof express one or more target antigens to which the one or more CARs and/or TCRs specifically bind.

36. The method of claim 35, wherein signaling by the one or more CARs and/or TCRs upon binding of the one or more target antigens expressed by the population of genetically engineered immune cells or a subset thereof by the one or more CARs and/or TCRs is reduced upon culture of the immune cells and the population of genetically engineered immune cells in the presence of the one or more TKIs.

37. The method of claim 36, wherein a reduction in signaling by the one or more CARs and/or TCRs reduces immune cell activation, differentiation, and/or fratricide by the population of genetically engineered immune cells or a subset thereof during expansion of the genetically engineered immune cells in culture compared to genetically engineered immune cells cultured in the absence of the one or more TKIs.

38. The method of any of claims 29-37, wherein the one or more target antigens comprise one or more endogenous gene products expressed by the immune cells.

39. The method of any of claims 35-38, wherein the one or more target antigens comprise CD2, CD5, CD7, CD4, CD8, CD3, CS1, CD38, CD99, CD30, 4-1BB, OX40, ICOS, CD26, CD6, TIGIT, PD-1, 2B4, LAG-3, MHC-I, MHC-II, peptide-MHC I, peptide-MHC II, Tim3, CTLA-4, CD112R, CD226, CD96, CD80, CD86, CD112, CD155, KIR2, KIR3, LILRB, CD28, CD40L, CD40, BTLA, GITR, VISTA, NKG2D ligands, or CD70.

40. The method of any of claims 29-37, wherein the one or more target antigens comprise one or more antigens acquired via trogocytosis and expressed by the immune cells.

41. The method of any one of claims 29-40, wherein the one or more CARs and/or TCRs comprise one or more antibodies or fragments thereof with specificity against the one or more target antigens.

42. The method of claim 41, wherein the antibodies or fragments thereof are scFv monoclonal antibodies, nanobodies/VHH-only sequences, fibronectin-derived binding domains, DARPINs, or natural ligands.

43. The method of any of claims 29-42, wherein the one or more CARs comprise a hinge or spacer comprising a sequence derived from IgG, CD3, CD4, CD5, CD8, CD9, CD16, CD22, CD28, CD33, CD37, CD45, CD64, CD80, CD86, CD134, CD137, CD154, 4-1BB, OX40, a T-cell receptor α or β chain, a CD3ξ chain, ICOS, or a combination thereof.

44. The composition of any of claims 29-43, wherein the one or more CARs comprise a hinge comprising an IgG4-derived sequence.

45. The method of any of claims 29-44, wherein the one or more CARs comprise a spacer comprising an IgG-derived sequence.

46. The method of any of claims 29-45, wherein the one or more CARs comprise a spacer comprising an IgG1-derived sequence.

47. The method of any of claims 29-46, wherein the one or more CARs comprise a C_{H}3 IgG1 spacer.

48. The method of any of claims 29-47, wherein the one or more CARs comprise one or more signaling domains from CD2, CD3ξ, CD3δ, CD3ε, CD3γ, Fc receptors, CD79a, CD79b, CLEC-2, CD7, LFA-1 (CD11a/CD18), CD27, CD28, CD30, CD40, 4-1BB (CD137), CD278, 2B4, DNAM-1, OX40, NKG2C, NKG2D, DAP10, DAP12, B7-1/CD80, CD28, 4-1BBL, B7-2/CD86, CTLA-4, B7-H1/PD-L1, ICOS, B7-H2, PD-1, B7-H3, PD-L2, B7-H4, PDCD6, HVEM, LIGHT, ICAM-1, BTLA, GITR, or a combination thereof.

49. The method of any of claims 29-48, wherein the one or more CARs comprise one or more signaling domains from CD3ξ, CD28, 4-1BB, or a combination thereof.

50. The method of any of claims 35-49, wherein the concentration of each of the one or more TKIs in culture is between 0.01 μM to 10 μM.

51. The method of any of claims 35-50, wherein the concentration of each of the one or more TKIs in culture is between 0.1 μM to 1 μM.

52. The method of any of claims 35-51, wherein the one or more TKIs comprise one or more Src kinase inhibitors.

53. The method of any of claims 35-52, wherein the one or more TKIs comprise dasatinib, ibrutinib, pp2, pazopanib, gefitinib, or a combination thereof.

54. The method of any of claims 35-53, wherein at least one of the one or more TKIs comprises dasatinib.

55. The method of any of claims 35-53, wherein at least one of the one or more TKIs comprises ibrutinib.

56. The method of any of claims 35-55, wherein the one or more TKIs comprise dasatinib and ibrutinib.

57. The method of any of claims 53-56, wherein the concentration of dasatinib in culture is 0.5 μM.

58. The method of any of claims 53-57, wherein the concentration of ibrutinib in culture is 0.2 μM.

59. The method of any of claims 35-58, wherein the one or more TKIs are added to the culture between 0 to 7 days before manipulation of the immune cell population to express the one or more CARs and/or TCRs.

60. The method of any of claims 35-59, wherein the one or more TKIs are added to the culture between 0 to 5 days before manipulation of the immune cell population to express the one or more CARs and/or TCRs.

61. The method of any of claims 35-60, wherein the one or more TKIs are added to the culture between 0 to 3 days before manipulation of the immune cell population to express the one or more CARs and/or TCRs.

62. The method of any of claims 35-61, wherein the immune cell population is manipulated to express the one or more CARs and/or TCRs with one or more expression vectors comprising one or more isolated nucleic acid sequences encoding the one or more CARs and/or TCRs.

63. The method of claim 62, wherein the one or more expression vectors are a lentiviral vector, a gamma-retroviral vector, adenoviral vector, adeno-associated viral vector, or a combination thereof.

64. The method of any of claims 35-63, further comprising expanding the population of immune cells in culture with the one or more TKIs prior to manipulating the population of immune cells to express one or more CARs and/or TCRs to produce the population of genetically engineered immune cells.

65. The method of any of claims 35-64, further comprising expanding the population of genetically engineered immune cells in culture with the one or more TKIs after manipulation of the population of immune cells to express one or more CARs and/or TCRs.

66. The method of any of claims 35-65, further comprising activating the population of immune cells prior to manipulating the population of immune cells to express one or more CARs and/or TCRs to produce the population of genetically engineered immune cells.

67. The method of any of claims 35-66, further comprising replenishing the one or more TKIs in the culture every 1, 2, 3, 4, or 5 days during culture.

68. The method of claim 67, wherein the one or more TKIs are replenished in the culture every day during culture.

69. The method of claim 67, wherein the one or more TKIs are replenished in the culture every 2 days during culture.

70. The method of claim 67, wherein the one or more TKIs are replenished in the culture every 3 days during culture.

71. The method of claim 67, wherein the one or more TKIs are replenished in the culture every 4 days during culture.

72. The method of claim 67, wherein the one or more TKIs are replenished in the culture every 5 days during culture.

73. The method of any of claims 35-72, further comprising depleting the population of genetically engineered immune cells of the one or more TKIs between 1 to 21 days after manipulation of the population of immune cells to express one or more CARs and/or TCRs to produce the genetically engineered immune cells.

74. The method of claim 73, wherein the population of genetically engineered immune cells is depleted of the one or more between 1 to 14 days after manipulation of the population of immune cells to express one or more CARs and/or TCRs to produce the population of genetically engineered immune cells.

75. The method of claim 73, wherein the population of genetically engineered immune cells is depleted of the one or more TKIs between 1 to 7 days after manipulation of the population of immune cells to express one or more CARs and/or TCRs to produce the population of genetically engineered immune cells.

76. The method of any of claims 73-75, where the population of genetically engineered immune cells is depleted of the one or more TKIs by sequential media washes of the population of genetically engineered immune cells.

77. The method of claim 76, wherein 2, 3, 4, 5, or 6 sequential washes of the population of genetically engineered immune cells are performed.

78. The method of claim 76 or claim 77, wherein 4 sequential washes of the population of genetically engineered cells are performed.

79. The method of any of claims 35-78, further comprising cryopreserving the population of genetically engineered cells.

80. The method of claim 79, wherein the population of genetically engineered cells are cryopreserved after depleting the population of genetically engineered cells of the one or more TKIs.

81. The method of any of claims 35-80, wherein one or more endogenous genes in the immune cells and/or the population of genetically engineered cells or a subset thereof are not inhibited.

82. A population of genetically engineered immune cells produced by the method of any of claims 35-81.

83. A method of killing a diseased cell, the method comprising contacting the diseased cell with the composition of any of claims 1-29 or the population of genetically engineered immune cells of claim 82.

84. The method of claim 83, wherein the diseased cell is a cancer cell.

85. The method of claim 84, wherein the cancer comprises T-ALL, T-cell lymphoma, leukemia, lymphoma, multiple myeloma, or a solid tumor.

86. The method of claim 83, wherein the diseased cell is a cell infected by an infectious disease microorganism.

87. The method of claim 83, wherein the diseased cell is a cell affected by an immune disorder.

88. A method of treating a cancer in a subject, the method comprising administering to a subject in need thereof a therapeutically effective amount of the composition of any of claims 1-29 or the population of genetically engineered immune cells of claim 82, wherein the one or more target antigens to which the one or more CARs and/or TCRs specifically bind are expressed by cancer cells in vivo, wherein the one or more CARs and/or TCRs specifically bind the one or more target antigens expressed by the cancer cells in vivo, and wherein binding of the one or more CARs and/or TCRs to the one or more target antigens expressed by the cancer cells in vivo results in elimination of the cancer cells.

89. The method of claim 88, wherein the amount of genetically engineered immune cells administered to the subject ranges from about 10⁴ up to about 10⁸ cells per kg body weight of the subject.

90. The method of claim 88 or claim 89, wherein the composition of any of claims 1-29 or the population of genetically engineered immune cells of claim 82 is administered to the subject by infusion, intravenously, intraperitoneally, intratracheally, intramuscularly, endoscopically, percutaneously, subcutaneously, regionally, intracranially, by direct injection, or by perfusion.

91. The method of any of claims 88-90, wherein the fratricidal activity of the population of genetically engineered immune cells is restored in vivo after substantial elimination of the cancer cells.

92. The method of claim 91, wherein restoration of the fratricidal activity of the population of genetically engineered immune cells results in elimination of the genetically engineered immune cells.

93. The method of any of claims 88-92, wherein the cancer is a myeloid malignancy, a lymphoid malignancy, and/or a solid tumor.

94. The method of any one of claims 88-93, wherein the cancer is T-cell acute lymphoblastic leukemia (T-ALL) or T-cell lymphoma.

95. A method of treating an immune disorder in a subject, the method comprising administering to a subject in need thereof a therapeutically effective amount of the composition of any of claims 1-29 or the population of genetically engineered immune cells of claim 82, wherein the one or more target antigens to which the one or more CARs and/or TCRs specifically bind are expressed by immune cells in vivo, wherein the one or more CARs and/or TCRs specifically bind the one or more target antigens expressed by the immune cells in vivo, and wherein binding of the one or more CARs and/or TCRs to the one or more target antigens expressed by the immune cells in vivo results in elimination of the immune cells.

96. The method of claim 95, wherein the amount of genetically engineered immune cells administered to the subject ranges from about 10⁴ up to about 10⁸ cells per kg body weight of the subject.

97. The method of claim 95 or claim 97, wherein the composition of any of claims 1-29 or the population of genetically engineered immune cells of claim 82 is administered to the subject by infusion, intravenously, intraperitoneally, intratracheally, intramuscularly, endoscopically, percutaneously, subcutaneously, regionally, intracranially, by direct injection, or by perfusion.

98. The method of any of claims 95-97, wherein the fratricidal activity of the population of genetically engineered immune cells is restored in vivo after substantial elimination of the immune cells.

99. The method of claim 98, wherein restoration of the fratricidal activity of the population of genetically engineered immune cells results in elimination of the genetically engineered immune cells.

100. The method of any of claims 95-99, wherein the immune disorder is an auto- or allo-immune disorder.

101. The method of any one of claims 95-100, wherein the auto- or allo-immune disorder is graft versus host disease, type 1 diabetes, multiple sclerosis, rheumatoid arthritis, psoriatic arthritis, systemic lupus erythematosus, inflammatory bowel disease, Guillain-Barre syndrome, chronic inflammatory demyelinating polyneuropathy, psoriasis, Graves' disease, Hashimoto's thyroiditis, myasthenia gravis, and/or vasculitis.

102. A composition comprising an effective amount of a population of genetically engineered immune cells comprising one or more chimeric antigen receptors (CARs) and/or T-cell receptors (TCRs), said composition produced by manipulating a population of immune cells in culture with one or more TKIs to express one or more CARs and/or TCRs to produce the population of genetically engineered immune cells,
wherein the population of genetically engineered immune cells or a subset thereof express one or more target antigens to which the one or more CARs and/or TCRs specifically bind,
wherein signaling by the one or more CARs and/or TCRs upon binding of the one or more target antigens expressed by the population of genetically engineered immune cells or a subset thereof by the one or more CARs and/or TCRs is reduced upon culture of the immune cells manipulated to express the one or more CARs and/or TCRs and/or the population of genetically engineered immune cells in the presence of one or more TKIs, and
wherein a reduction in signaling by the one or more CARs and/or TCRs reduces immune cell activation, differentiation, and/or fratricide by the population of genetically engineered immune cells or a subset thereof compared to genetically engineered immune cells cultured in the absence of the one or more TKIs.

103. The composition of claim 102, wherein the immune cells comprise T-cells, Natural Killer (NK) cells, myeloid cells, B-cells, or a mixture thereof.

104. The composition of claim 102 or claim 103, wherein the immune cells comprise T-cells.

105. The composition of claim 102 or claim 103, wherein the immune cells comprise NK cells.

106. The composition of claim 102 or claim 103, wherein the immune cells comprise myeloid cells.

107. The composition of claim 102 or claim 103, wherein the immune cells comprise B-cells.

108. The composition of any of claims 102-107, wherein the one or more target antigens comprise one or more endogenous gene products expressed by the immune cells.

109. The composition of claim 108, wherein the one or more target antigens comprise CD2, CD5, CD7, CD4, CD8, CD3, CS1, CD38, CD99, CD30, 4-1BB, OX40, ICOS, CD26, CD6, TIGIT, PD-1, 2B4, LAG-3, MHC-I, MHC-II, peptide-MHC I, peptide-MHC II, Tim3, CTLA-4, CD112R, CD226, CD96, CD80, CD86, CD112, CD155, KIR2, KIR3, LILRB, CD28, CD40L, CD40, BTLA, GITR, VISTA, NKG2D ligands, or CD70.

110. The composition of any of claims 102-105, wherein the one or more target antigens comprise one or more antigens acquired via trogocytosis and expressed by the immune cells.

111. The composition of any of claims 102-110, wherein the one or more CARs and/or TCRs comprise one or more antibodies or fragments thereof with specificity against the one or more target antigens.

112. The composition of claim 111, wherein the antibodies or fragments thereof are scFv monoclonal antibodies, nanobodies/VHH-only sequences, fibronectin-derived binding domains, DARPINs, or natural ligands.

113. The composition of any of claims 102-112, wherein the one or more CARs comprise a hinge or spacer comprising a sequence derived from IgG, CD3, CD4, CD5, CD8, CD9, CD16, CD22, CD28, CD33, CD37, CD45, CD64, CD80, CD86, CD134, CD137, CD154, 4-1BB, OX40, a T-cell receptor α or β chain, a CD3ξ chain, ICOS, or a combination thereof.

114. The composition of any of claims 102-113, wherein the one or more CARs comprise a hinge comprising an IgG4-derived sequence.

115. The composition of any of claims 102-114, wherein the one or more CARs comprise a spacer comprising an IgG-derived sequence.

116. The composition of any of claims 102-115, wherein the one or more CARs comprise a spacer comprising an IgG1-derived sequence.

117. The composition of any of claims 102-116, wherein the one or more CARs comprise a C_{H}3 IgG1 spacer.

118. The composition of any of claims 102-117, wherein the one or more CARs comprise one or more signaling domains from CD2, CD3ξ, CD3δ, CD3ε, CD3γ, Fc receptors, CD79a, CD79b, CLEC-2, CD7, LFA-1 (CD11a/CD18), CD27, CD28, CD30, CD40, 4-1BB (CD137), CD278, 2B4, DNAM-1, OX40, NKG2C, NKG2D, DAP10, DAP12, B7-1/CD80, CD28, 4-1BBL, B7-2/CD86, CTLA-4, B7-H1/PD-L1, ICOS, B7-H2, PD-1, B7-H3, PD-L2, B7-H4, PDCD6, HVEM, LIGHT, ICAM-1, BTLA, GITR, or a combination thereof.

119. The composition of any of claims 102-118, wherein the one or more CARs comprise one or more signaling domains from CD3ξ, CD28, 4-1BB, or a combination thereof.

120. The composition of any of claims 102-119, wherein the one or more CARs and/or TCRs are encoded by one or more isolated nucleic acid sequences.

121. The composition of claim 120, wherein the one or more isolated nucleic acid sequences are comprised in one or more expression vectors.

122. The composition of claim 121, wherein the one or more expression vectors are a lentiviral vector, a gamma-retroviral vector, adenoviral vector, an adeno-associated viral vector, or a combination thereof.

123. The composition of any of claims 102-122, wherein the concentration of each of the one or more TKIs in culture is between 0.01 μM to 10 μM.

124. The composition of any of claims 102-123, wherein the concentration of each of the one or more TKIs in culture is between 0.1 μM to 1 μM.

125. The composition of any of claims 102-124, wherein the one or more TKIs comprise dasatinib, ibrutinib, pp2, pazopanib, gefitinib, or a combination thereof.

126. The composition of any of claims 102-125, wherein at least one of the one or more TKIs comprises dasatinib.

127. The composition of any of claims 102-125, wherein at least one of the one or more TKIs comprises ibrutinib.

128. The composition of any of claims 102-127, wherein the one or more TKIs comprise dasatinib and ibrutinib.

129. The composition of any of claims 125-128, wherein the concentration of dasatinib in culture is 0.5 μM.

130. The composition of any of claims 125-129, wherein the concentration of ibrutinib in culture is 0.2 μM.

131. The composition of any of claims 102-130, wherein the one or more TKIs are added to the culture between 0 to 7 days before manipulation of the immune cell population to express the one or more CARs and/or TCRs.

132. The composition of any of claims 102-131, wherein the one or more TKIs are added to the culture between 0 to 5 days before manipulation of the immune cell population to express the one or more CARs and/or TCRs.

133. The composition of any of claims 102-132, wherein the one or more TKIs are added to the culture between 0 to 3 days before manipulation of the immune cell population to express the one or more CARs and/or TCRs.

134. The composition of any of claims 102-133, wherein the immune cell population is manipulated to express the one or more CARs and/or TCRs with one or more expression vectors comprising one or more isolated nucleic acid sequences encoding the one or more CARs and/or TCRs.

135. The composition of claim 134, wherein the one or more expression vectors are a lentiviral vector, a gamma-retroviral vector, adenoviral vector, adeno-associated viral vector, or a combination thereof.

136. The composition of any of claims 102-135, further comprising expanding the population of immune cells in culture with the one or more TKIs prior to manipulating the population of immune cells to express one or more CARs and/or TCRs to produce the population of genetically engineered immune cells.

137. The composition of any of claims 102-136, further comprising expanding the population of genetically engineered immune cells in culture with the one or more TKIs after manipulation of the population of immune cells to express one or more CARs and/or TCRs.

138. The composition of any of claims 102-137, further comprising activating the population of immune cells prior to manipulating the population of immune cells to express one or more CARs and/or TCRs to produce the population of genetically engineered immune cells.

139. The composition of any of claims 102-138, further comprising replenishing the one or more TKIs in the culture every 1, 2, 3, 4, or 5 days during culture.

140. The composition of claim 139, wherein the one or more TKIs are replenished in the culture every day during culture.

141. The composition of claim 139, wherein the one or more TKIs are replenished in the culture every 2 days during culture.

142. The composition of claim 139, wherein the one or more TKIs are replenished in the culture every 3 days during culture.

143. The composition of claim 139, wherein the one or more TKIs are replenished in the culture every 4 days during culture.

144. The composition of claim 139, wherein the one or more TKIs are replenished in the culture every 5 days during culture.

145. The composition of any of claims 102-144, further comprising depleting the population of genetically engineered immune cells of the one or more TKIs between 1 to 21 days after manipulation of the population of immune cells to express one or more CARs and/or TCRs to produce the population of genetically engineered immune cells.

146. The composition of claim 145, wherein the population of genetically engineered immune cells is depleted of the one or more TKIs between 1 to 14 days after manipulation of the population of immune cells to express one or more CARs and/or TCRs to produce population of the genetically engineered immune cells.

147. The composition of claim 145, wherein the population of genetically engineered immune cells is depleted of the one or more TKIs between 1 to 7 days after manipulation of the population of immune cells to express one or more CARs and/or TCRs to produce the population of genetically engineered immune cells.

148. The composition of any of claims 145-147, where the population of genetically engineered immune cells is depleted of the one or more kinase inhibitors by sequential media washes of the population of genetically engineered immune cells.

149. The composition of claim 148, wherein 2, 3, 4, 5, or 6 sequential washes of the population of genetically engineered immune cells are performed.

150. The composition of claim 148 or claim 149, wherein 4 sequential washes of the population of genetically engineered immune cells are performed.

151. The composition of any of claims 102-150, further comprising cryopreserving the population of genetically engineered immune cells.

152. The composition of claim 151, wherein the population of genetically engineered immune cells is cryopreserved after depleting the population of genetically engineered immune cells of the one or more TKIs.

153. The composition of any of claims 102-152, wherein one or more endogenous genes in the immune cells and/or the genetically engineered immune cells are not inhibited.

154. The composition of any of claims 102-153, further comprising a pharmaceutically acceptable carrier.


SEQUENCE LISTING (48 sequences)

SEQ ID NO: 1 - DNA, 63 bp, Artificial Sequence
  Synthetic Polynucleotide
atggccttaccagtgaccgccttgctcctgccgctggccttgctgctccacgccgccaggccg

SEQ ID NO: 2 - protein, 21 aa, Artificial Sequence
  Synthetic Polypeptide
MALPVTALLLPLALLLHAARP

SEQ ID NO: 3 - DNA, 57 bp, Artificial Sequence
  Synthetic Polynucleotide
atggagtttgggctgagctggctttttcttgtggctattttaaaaggtgtccagtgc

SEQ ID NO: 4 - protein, 19 aa, Artificial Sequence
  Synthetic Polypeptide
MEFGLSWLFLVAILKGVQC

SEQ ID NO: 5 - DNA, 809 bp, Artificial Sequence
  Synthetic Polynucleotide
atggagtttgggctgagctggctttttcttgtggctattttaaaaggtgtccagtgcatcgatgccatgggcaacatccagctggtgcagagcggccctgagctgaagaaacccggcgagacagtgaagatcagctgcaaggccagcggctacaccttcaccaactacggcatgaactgggtgaaacaggccccaggcaagggcctgcggtggatgggctggatcaacacccacaccggcgagcccacctacgccgacgacttcaagggcagattcgccttcagcctggaaaccagcgccagcaccgcctacctgcagatcaacaacctgaagaacgaggacaccgccacctatttctgcaccagacggggctacgactggtacttcgacgtgtggggagccggcaccaccgtgaccgtgtctagcggaggcggaggatctggcggagggggatcaggcggcggaggcagcgacatcaagatgacccagagccccagctctatgtacgccagcctgggcgagcgcgtgaccatcacatgcaaggcctcccaggacatcaacagctacctgagctggttccaccacaagcccggcaagagccccaagaccctgatctaccgggccaaccggctggtggacggcgtgccaagcagattcagcggcagcggctccggccaggactacagcctgaccatcagcagcctggactacgaggacatgggcatctactactgccagcagtacgacgagagcccctggaccttcggaggcggcaccaagctggaaatgaagggcagcggggatcccgc

SEQ ID NO: 6 - protein, 270 aa, Artificial Sequence
  Synthetic Polypeptide
MEFGLSWLFLVAILKGVQCIDAMGNIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSGGGGSGGGGSGGGGSDIKMTQSPSSMYASLGERVTITCKASQDINSYLSWFHHKPGKSPKTLIYRANRLVDGVPSRFSGSGSGQDYSLTISSLDYEDMGIYYCQQYDESPWTFGGGTKLEMKGSGDPA

SEQ ID NO: 7 - DNA, 723 bp, Artificial Sequence
  Synthetic Polynucleotide
caggtgaagctgcaggagtcagggggaggcttagtgaagcctggagggtccctgaaactctcctgtgcagcctctggattcactttcagtagctatgcaatgtcttgggttcgccagactccggagaagaggctggagtgggtcgcaaccattagtagtggtggtagttacacctactatccagacagtgtgaaggggcgattcaccatctccagagacaatgccaagaacaccctgtacctgcaaatgagcagtctgaggtctgaggacacggccatgtattactgtgcaagacaggatggttactacccgggctggtttgctaactgggggcaagggaccacggtcaccgtctcctcaggtggaggcggttcaggcggaggtggctctggcggtggcggatcggacatcgagctcactcagtctccagcaatcatgtctgcatctctaggggaggagatcaccctaacctgcagtgccagctccagtgtaagttacatgcactggtaccagcagaagtcaggcacttctcccaaactcttgatttatagcacatccaacctggcttctggagtcccttctcgcttcagtggcagtgggtctgggaccttttattctctcacaatcagcagtgtggaggctgaagatgctgccgattattactgccatcagtggagtagttacacgttcggagggggcaccaagctggaaatcaaacgggcg

SEQ ID NO: 8 - protein, 242 aa, Artificial Sequence
  Synthetic Polypeptide
PQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSGGGGSGGGGSGGGGSDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRA

SEQ ID NO: 9 - DNA, 845 bp, Artificial Sequence
  Synthetic Polynucleotide
atggccctgcctgtgaccgctctgctgctgcctctggcactgctgctgcacgctgctagacctggcgctcagcctgctatggccgcctacaaggacatccagatgacccagaccaccagcagcctgtctgccagcctgggcgacagagtgaccatcagctgtagcgccagccagggcatcagcaactacctgaactggtatcagcagaaacccgacggcaccgtgaagctgctgatctactacaccagctccctgcacagcggcgtgcccagcagattttctggcagcggctccggcaccgactacagcctgaccatctccaacctggaacccgaggatatcgccacctactactgccagcagtacagcaagctgccctacaccttcggcggaggcaccaagctggaaatcaagaggggaggcggaggaagcggaggcggtggatctggtggtggcggttctggcggaggtggaagcgaagtgcagctggtggaatctggcggcggactggtcaagcctggcggctctctgaaactgagctgtgccgcctctggcctgaccttcagcagctacgctatgagctgggtgcgccagacccccgagaagagactggaatgggtggccagcatcagcagcggcggctttacctactaccccgacagcgtgaagggccggttcaccatcagccgggacaacgcccggaacatcctgtacctgcagatgagcagcctgcggagcgaggacaccgccatgtactactgcgccagggatgaagtgcggggctacctggatgtgtggggagccggaacaaccgtgaccgtgtctagtgccagcggagcggatcc

SEQ ID NO: 10 - protein, 283 aa, Artificial Sequence
  Synthetic Polypeptide
MALPVTALLLPLALLLHAARPGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSASGADPA

SEQ ID NO: 11 - DNA, 809 bp, Artificial Sequence
  Synthetic Polynucleotide
atggccttaccagtgaccgccttgctcctgccgctggccttgctgctccacgccgccaggccgcaggtccagctgcaggagtctggggctgaactggtgaagcctggggcttcagtgaagctgtcctgcaaggcttctggctacaccttcacgagctactggatgcactgggtgaagcagaggcctggacaaggccttgagtggattggaaagattaatcctagcaacggtcgtactaactacaatgagaagttcaagagcaaggccacactgactgtagacaaatcctccagcacagcctacatgcaactcagcagcctgacatctgaggactctgcggtctattactgtgcaagagggggagtctactatgacctttattactatgctctggactactggggccaaggcaccacggtcaccgtctcctcaggtggaggcggttcaggcggaggtggctctggcggtggcggatcggacatcgagctcactcagtctccagccaccctgtctgtgactccaggagatagcgtcagtctttcctgcagggccagccaaagtattagcaacaacctacactggtatcaacaaaaatcacatgagtctccaaggcttctcatcaagtctgcttcccagtccatctctggaatcccctccaggttcagtggcagtggatcagggacagatttcactctcagtatcaacagtgtggagactgaagattttggaatgtatttctgtcaacagagtaacagctggccgtacacgttcggaggggggacaaagttggaaataaaacgggcggatcc

SEQ ID NO: 12 - protein, 271 aa, Artificial Sequence
  Synthetic Polypeptide
MALPVTALLLPLALLLHAARPQVQLQESGAELVKPGASVKLSCKASGYTFTSYWMHWVKQRPGQGLEWIGKINPSNGRTNYNEKFKSKATLTVDKSSSTAYMQLSSLTSEDSAVYYCARGGVYYDLYYYALDYWGQGTTVTVSSGGGGSGGGGSGGGGSDIELTQSPATLSVTPGDSVSLSCRASQSISNNLHWYQQKSHESPRLLIKSASQSISGIPSRFSGSGSGTDFTLSINSVETEDFGMYFCQQSNSWPYTFGGGTKLEIKRADPA

SEQ ID NO: 13 - DNA, 738 bp, Artificial Sequence
  Synthetic Polynucleotide
gatgttgttcttactcagactccaccaactttgttggcaacaattgggcaaagtgtgtcaattagttgcagatcaagccaaagtctcttgcacagtagcggaaatacctatctgaactggctgttgcagcggactgggcaatccccgcaaccgctcatatacctggtaagcaagctagagtcaggggtgccgaatcgcttctccggatccggtagtggtacggatttcacgctgaagataagcggagtggaagcggaagacttgggcgtgtactactgtatgcagttcacacactatccctacacttttggggcgggtactaaacttgagcttaagtctggaggcggtggatctggcggtggaggtagcggaggaggcggtagcgaagtgcaattgcagcagtcagggccagagctgcaaagacctggtgccagcgtgaagttgtcctgtaaagcctccggttatatcttcacagagtactatatgtactgggttaagcaacgcccaaaacaaggcctggagcttgtgggccgaatcgaccccgaagatggttctattgactacgtagagaagttcaagaaaaaggcaacactcactgcggacactagttcaaacactgcctacatgcagctctctagcctgacatccgaagacaccgccacgtatttttgcgcacgaggtaaattcaactatcgcttcgcatactgggggcagggtactctcgtcaccgtctcctca

SEQ ID NO: 14 - protein, 247 aa, Artificial Sequence
  Synthetic Polypeptide
DVVLTQTPPTLLATIGQSVSISCRSSQSLLHSSGNTYLNWLLQRTGQSPQPLIYLVSKLESGVPNRFSGSGSGTDFTLKISGVEAEDLGVYYCMQFTHYPYTFGAGTKLELKSGGGGSGGGGSGGGGSEVQLQQSGPELQRPGASVKLSCKASGYIFTEYYMYWVKQRPKQGLELVGRIDPEDGSIDYVEKFKKKATLTADTSSNTAYMQLSSLTSEDTATYFCARGKFNYRFAYWGQGTLVTVSSA

SEQ ID NO: 15 - DNA, 741 bp, Artificial Sequence
  Synthetic Polynucleotide
gaagtgcaattgcagcagtcagggccagagctgcaaagacctggtgccagcgtgaagttgtcctgtaaagcctccggttatatcttcacagagtactatatgtactgggttaagcaacgcccaaaacaaggcctggagcttgtgggccgaatcgaccccgaagatggttctattgactacgtagagaagttcaagaaaaaggcaacactcactgcggacactagttcaaacactgcctacatgcagctctctagcctgacatccgaagacaccgccacgtatttttgcgcacgaggtaaattcaactatcgcttcgcatactgggggcagggtactctcgtcaccgtctcctcatctggaggcggtggatctggcggtggaggtagcggaggaggcggtagcgatgttgttcttactcagactccaccaactttgttggcaacaattgggcaaagtgtgtcaattagttgcagatcaagccaaagtctcttgcacagtagcggaaatacctatctgaactggctgttgcagcggactgggcaatccccgcaaccgctcatatacctggtaagcaagctagagtcaggggtgccgaatcgcttctccggatccggtagtggtacggatttcacgctgaagataagcggagtggaagcggaagacttgggcgtgtactactgtatgcagttcacacactatccctacacttttggggcgggtactaaacttgagcttaaggcc

SEQ ID NO: 16 - protein, 247 aa, Artificial Sequence
  Synthetic Polypeptide
EVQLQQSGPELQRPGASVKLSCKASGYIFTEYYMYWVKQRPKQGLELVGRIDPEDGSIDYVEKFKKKATLTADTSSNTAYMQLSSLTSEDTATYFCARGKFNYRFAYWGQGTLVTVSSSGGGGSGGGGSGGGGSDVVLTQTPPTLLATIGQSVSISCRSSQSLLHSSGNTYLNWLLQRTGQSPQPLIYLVSKLESGVPNRFSGSGSGTDFTLKISGVEAEDLGVYYCMQFTHYPYTFGAGTKLELKA

SEQ ID NO: 17 - DNA, 741 bp, Artificial Sequence
  Synthetic Polynucleotide
gcccagccggccatggccaaggtccagctgcaggagtcaggacctagcctagtgcagccctcacagcgcctgtccataacctgcacagtctctggtttctcattaattagttatggtgtacactgggttcgccagtctccaggaaagggtctggagtggctgggagtgatatggagaggtggaagcacagactacaatgcagctttcatgtccagactgagcatcaccaaggacaactccaagagccaagttttctttaaaatgaacagtctgcaagctgatgacactgccatatacttctgtgccaaaaccttgattacgacgggctatgctatggactactggggccaagggaccacggtcaccgtctcctcaggtggaggcggttcaggcggaggtggctctggcggtggcggatcggacatcgagctcactcagtctccatcctccttttctgtatctctaggagacagagtcaccattacttgcaaggcaagtgaggacatatataatcggttagcctggtatcagcagaaaccaggaaatgctcctaggctcttaatatctggtgcaaccagtttggaaactggggttccttcaagattcagtggcagtggatctggaaaggattacactctcagcattaccagtcttcagactgaagatgttgctacttattactgtcaacagtattggagtactcctacgttcggtggagggaccaagctggaaatcaaacgg

SEQ ID NO: 18 - protein, 247 aa, Artificial Sequence
  Synthetic Polypeptide
AQPAMAKVQLQESGPSLVQPSQRLSITCTVSGFSLISYGVHWVRQSPGKGLEWLGVIWRGGSTDYNAAFMSRLSITKDNSKSQVFFKMNSLQADDTAIYFCAKTLITTGYAMDYWGQGTTVTVSSGGGGSGGGGSGGGGSDIELTQSPSSFSVSLGDRVTITCKASEDIYNRLAWYQQKPGNAPRLLISGATSLETGVPSRFSGSGSGKDYTLSITSLQTEDVATYYCQQYWSTPTFGGGTKLEIKR

SEQ ID NO: 19 - DNA, 87 bp, Artificial Sequence
  Synthetic Polynucleotide
ttttgggtgctggtggtggttggtggagtcctggcttgctatagcttgctagtaacagtggcctttattattttctgggtgaggagt

SEQ ID NO: 20 - protein, 29 aa, Artificial Sequence
  Synthetic Polypeptide
FWVLVVVGGVLACYSLLVTVAFIIFWVRS

SEQ ID NO: 21 - DNA, 117 bp, Artificial Sequence
  Synthetic Polynucleotide
aagaggagcaggctcctgcacagtgactacatgaacatgactccccgccgccccgggcccacccgcaagcattaccagccctatgccccaccacgcgacttcgcagcctatcgctcc

SEQ ID NO: 22 - protein, 39 aa, Artificial Sequence
  Synthetic Polypeptide
KRSRLLHSDYMNMTPRRPGPTRKHYQPYAPPRDFAAYRS

SEQ ID NO: 23 - DNA, 126 bp, Artificial Sequence
  Synthetic Polynucleotide
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 24 - protein, 42 aa, Artificial Sequence
  Synthetic Polypeptide
KRGRKKLLYIFKQPFMRPVQTTQEEDGCSCRFPEEEEGGCEL

SEQ ID NO: 25 - DNA, 336 bp, Artificial Sequence
  Synthetic Polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 26 - protein, 112 aa, Artificial Sequence
  Synthetic Polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 742 bp, Artificial Sequence
  Synthetic Polynucleotide
gtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaatt

SEQ ID NO: 28 - protein, 246 aa, Artificial Sequence
  Synthetic Polypeptide
TVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 29 - DNA, 364 bp, Artificial Sequence
  Synthetic Polynucleotide
gagtctaaatatggcccaccttgcccaccgtgcccagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaacgcctacacgcagaagagcctctccctgtctccgggtaaaaaagatc

SEQ ID NO: 30 - protein, 123 aa, Artificial Sequence
  Synthetic Polypeptide
ESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKDPK

SEQ ID NO: 31 - DNA, 187 bp, Artificial Sequence
  Synthetic Polynucleotide
ctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcg

SEQ ID NO: 32 - protein, 63 aa, Artificial Sequence
  Synthetic Polypeptide
LSNSIMYFSHFVPVFLPAKPTTTPAPRPPTPAPTIASQPLSLRPEACRPAAGGAVHTRGLDFA

SEQ ID NO: 33 - DNA, 1719 bp, Artificial Sequence
  Synthetic Polynucleotide
atggagtttgggctgagctggctttttcttgtggctattttaaaaggtgtccagtgcatcgatgccatgggcaacatccagctggtgcagagcggccctgagctgaagaaacccggcgagacagtgaagatcagctgcaaggccagcggctacaccttcaccaactacggcatgaactgggtgaaacaggccccaggcaagggcctgcggtggatgggctggatcaacacccacaccggcgagcccacctacgccgacgacttcaagggcagattcgccttcagcctggaaaccagcgccagcaccgcctacctgcagatcaacaacctgaagaacgaggacaccgccacctatttctgcaccagacggggctacgactggtacttcgacgtgtggggagccggcaccaccgtgaccgtgtctagcggaggcggaggatctggcggagggggatcaggcggcggaggcagcgacatcaagatgacccagagccccagctctatgtacgccagcctgggcgagcgcgtgaccatcacatgcaaggcctcccaggacatcaacagctacctgagctggttccaccacaagcccggcaagagccccaagaccctgatctaccgggccaaccggctggtggacggcgtgccaagcagattcagcggcagcggctccggccaggactacagcctgaccatcagcagcctggactacgaggacatgggcatctactactgccagcagtacgacgagagcccctggaccttcggaggcggcaccaagctggaaatgaagggcagcggggatcccgccgagtctaaatatggcccaccttgcccaccgtgcccagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaacgcctacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgc

SEQ ID NO: 34 - protein, 573 aa, Artificial Sequence
  Synthetic Polypeptide
MEFGLSWLFLVAILKGVQCIDAMGNIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSGGGGSGGGGSGGGGSDIKMTQSPSSMYASLGERVTITCKASQDINSYLSWFHHKPGKSPKTLIYRANRLVDGVPSRFSGSGSGQDYSLTISSLDYEDMGIYYCQQYDESPWTFGGGTKLEMKGSGDPAESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 1704 bp, Artificial Sequence
  Synthetic Polynucleotide
atggccttaccagtgaccgccttgctcctgccgctggccttgctgctccacgccgccaggccgcaggtgaagctgcaggagtcagggggaggcttagtgaagcctggagggtccctgaaactctcctgtgcagcctctggattcactttcagtagctatgcaatgtcttgggttcgccagactccggagaagaggctggagtgggtcgcaaccattagtagtggtggtagttacacctactatccagacagtgtgaaggggcgattcaccatctccagagacaatgccaagaacaccctgtacctgcaaatgagcagtctgaggtctgaggacacggccatgtattactgtgcaagacaggatggttactacccgggctggtttgctaactgggggcaagggaccacggtcaccgtctcctcaggtggaggcggttcaggcggaggtggctctggcggtggcggatcggacatcgagctcactcagtctccagcaatcatgtctgcatctctaggggaggagatcaccctaacctgcagtgccagctccagtgtaagttacatgcactggtaccagcagaagtcaggcacttctcccaaactcttgatttatagcacatccaacctggcttctggagtcccttctcgcttcagtggcagtgggtctgggaccttttattctctcacaatcagcagtgtggaggctgaagatgctgccgattattactgccatcagtggagtagttacacgttcggagggggcaccaagctggaaatcaaacgggcggatcccgccgagtctaaatatggcccaccttgcccaccgtgcccagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaacgcctacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 36 - protein, 568 aa, Artificial Sequence
  Synthetic Polypeptide
MALPVTALLLPLALLLHAARPQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSGGGGSGGGGSGGGGSDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRADPAESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - DNA, 1758 bp, Artificial Sequence
  Synthetic Polynucleotide
atggccctgcctgtgaccgctctgctgctgcctctggcactgctgctgcacgctgctagacctggcgctcagcctgctatggccgcctacaaggacatccagatgacccagaccaccagcagcctgtctgccagcctgggcgacagagtgaccatcagctgtagcgccagccagggcatcagcaactacctgaactggtatcagcagaaacccgacggcaccgtgaagctgctgatctactacaccagctccctgcacagcggcgtgcccagcagattttctggcagcggctccggcaccgactacagcctgaccatctccaacctggaacccgaggatatcgccacctactactgccagcagtacagcaagctgccctacaccttcggcggaggcaccaagctggaaatcaagaggggaggcggaggaagcggaggcggtggatctggtggtggcggttctggcggaggtggaagcgaagtgcagctggtggaatctggcggcggactggtcaagcctggcggctctctgaaactgagctgtgccgcctctggcctgaccttcagcagctacgctatgagctgggtgcgccagacccccgagaagagactggaatgggtggccagcatcagcagcggcggctttacctactaccccgacagcgtgaagggccggttcaccatcagccgggacaacgcccggaacatcctgtacctgcagatgagcagcctgcggagcgaggacaccgccatgtactactgcgccagggatgaagtgcggggctacctggatgtgtggggagccggaacaaccgtgaccgtgtctagtgccagcggagcggatcccgccgagtctaaatatggcccaccttgcccaccgtgcccagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaacgcctacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 38 - protein, 586 aa, Artificial Sequence
  Synthetic Polypeptide
MALPVTALLLPLALLLHAARPGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSASGADPAESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - DNA, 1722 bp, Artificial Sequence
  Synthetic Polynucleotide
atggccttaccagtgaccgccttgctcctgccgctggccttgctgctccacgccgccaggccgcaggtccagctgcaggagtctggggctgaactggtgaagcctggggcttcagtgaagctgtcctgcaaggcttctggctacaccttcacgagctactggatgcactgggtgaagcagaggcctggacaaggccttgagtggattggaaagattaatcctagcaacggtcgtactaactacaatgagaagttcaagagcaaggccacactgactgtagacaaatcctccagcacagcctacatgcaactcagcagcctgacatctgaggactctgcggtctattactgtgcaagagggggagtctactatgacctttattactatgctctggactactggggccaaggcaccacggtcaccgtctcctcaggtggaggcggttcaggcggaggtggctctggcggtggcggatcggacatcgagctcactcagtctccagccaccctgtctgtgactccaggagatagcgtcagtctttcctgcagggccagccaaagtattagcaacaacctacactggtatcaacaaaaatcacatgagtctccaaggcttctcatcaagtctgcttcccagtccatctctggaatcccctccaggttcagtggcagtggatcagggacagatttcactctcagtatcaacagtgtggagactgaagattttggaatgtatttctgtcaacagagtaacagctggccgtacacgttcggaggggggacaaagttggaaataaaacgggcggatcccgccgagtctaaatatggcccaccttgcccaccgtgcccagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaacgcctacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 40 - protein, 574 aa, Artificial Sequence
  Synthetic Polypeptide
MALPVTALLLPLALLLHAARPQVQLQESGAELVKPGASVKLSCKASGYTFTSYWMHWVKQRPGQGLEWIGKINPSNGRTNYNEKFKSKATLTVDKSSSTAYMQLSSLTSEDSAVYYCARGGVYYDLYYYALDYWGQGTTVTVSSGGGGSGGGGSGGGGSDIELTQSPATLSVTPGDSVSLSCRASQSISNNLHWYQQKSHESPRLLIKSASQSISGIPSRFSGSGSGTDFTLSINSVETEDFGMYFCQQSNSWPYTFGGGTKLEIKRADPAESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - DNA, 1713 bp, Artificial Sequence
  Synthetic Polynucleotide
atggccttaccagtgaccgccttgctcctgccgctggccttgctgctccacgccgccaggccggatgttgttcttactcagactccaccaactttgttggcaacaattgggcaaagtgtgtcaattagttgcagatcaagccaaagtctcttgcacagtagcggaaatacctatctgaactggctgttgcagcggactgggcaatccccgcaaccgctcatatacctggtaagcaagctagagtcaggggtgccgaatcgcttctccggatccggtagtggtacggatttcacgctgaagataagcggagtggaagcggaagacttgggcgtgtactactgtatgcagttcacacactatccctacacttttggggcgggtactaaacttgagcttaagtctggaggcggtggatctggcggtggaggtagcggaggaggcggtagcgaagtgcaattgcagcagtcagggccagagctgcaaagacctggtgccagcgtgaagttgtcctgtaaagcctccggttatatcttcacagagtactatatgtactgggttaagcaacgcccaaaacaaggcctggagcttgtgggccgaatcgaccccgaagatggttctattgactacgtagagaagttcaagaaaaaggcaacactcactgcggacactagttcaaacactgcctacatgcagctctctagcctgacatccgaagacaccgccacgtatttttgcgcacgaggtaaattcaactatcgcttcgcatactgggggcagggtactctcgtcaccgtctcctcagagtctaaatatggcccaccttgcccaccgtgcccagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaacgcctacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 42 - protein, 570 aa, Artificial Sequence
  Synthetic Polypeptide
MALPVTALLLPLALLLHAARPDVVLTQTPPTLLATIGQSVSISCRSSQSLLHSSGNTYLNWLLQRTGQSPQPLIYLVSKLESGVPNRFSGSGSGTDFTLKISGVEAEDLGVYYCMQFTHYPYTFGAGTKLELKSGGGGSGGGGSGGGGSEVQLQQSGPELQRPGASVKLSCKASGYIFTEYYMYWVKQRPKQGLELVGRIDPEDGSIDYVEKFKKKATLTADTSSNTAYMQLSSLTSEDTATYFCARGKFNYRFAYWGQGTLVTVSSESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - DNA, 1710 bp, Artificial Sequence
  Synthetic Polynucleotide
atggccttaccagtgaccgccttgctcctgccgctggccttgctgctccacgccgccaggccggaagtgcaattgcagcagtcagggccagagctgcaaagacctggtgccagcgtgaagttgtcctgtaaagcctccggttatatcttcacagagtactatatgtactgggttaagcaacgcccaaaacaaggcctggagcttgtgggccgaatcgaccccgaagatggttctattgactacgtagagaagttcaagaaaaaggcaacactcactgcggacactagttcaaacactgcctacatgcagctctctagcctgacatccgaagacaccgccacgtatttttgcgcacgaggtaaattcaactatcgcttcgcatactgggggcagggtactctcgtcaccgtctcctcatctggaggcggtggatctggcggtggaggtagcggaggaggcggtagcgatgttgttcttactcagactccaccaactttgttggcaacaattgggcaaagtgtgtcaattagttgcagatcaagccaaagtctcttgcacagtagcggaaatacctatctgaactggctgttgcagcggactgggcaatccccgcaaccgctcatatacctggtaagcaagctagagtcaggggtgccgaatcgcttctccggatccggtagtggtacggatttcacgctgaagataagcggagtggaagcggaagacttgggcgtgtactactgtatgcagttcacacactatccctacacttttggggcgggtactaaacttgagcttaaggagtctaaatatggcccaccttgcccaccgtgcccagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaacgcctacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgc

SEQ ID NO: 44 - protein, 570 aa, Artificial Sequence
  Synthetic Polypeptide
MALPVTALLLPLALLLHAARPEVQLQQSGPELQRPGASVKLSCKASGYIFTEYYMYWVKQRPKQGLELVGRIDPEDGSIDYVEKFKKKATLTADTSSNTAYMQLSSLTSEDTATYFCARGKFNYRFAYWGQGTLVTVSSSGGGGSGGGGSGGGGSDVVLTQTPPTLLATIGQSVSISCRSSQSLLHSSGNTYLNWLLQRTGQSPQPLIYLVSKLESGVPNRFSGSGSGTDFTLKISGVEAEDLGVYYCMQFTHYPYTFGAGTKLELKESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - DNA, 20 bp, Artificial Sequence
  Synthetic Polynucleotide
agagctacgagctgcctgac

SEQ ID NO: 46 - DNA, 18 bp, Artificial Sequence
  Synthetic Polynucleotide
ggatgccacaggactcca

SEQ ID NO: 47 - DNA, 22 bp, Artificial Sequence
  Synthetic Polynucleotide
ccaggacaacctgactatcacc

SEQ ID NO: 48 - DNA, 19 bp, Artificial Sequence
  Synthetic Polynucleotide
agcatctgtgccatccttg